SEQUENCE LISTING (99 sequences)

SEQ ID NO: 1 - protein, 332 aa, Arabidopsis thaliana
MPSEAESSANSAPATPPPPPNFWGTMPEEEYYTSQGVRNSKSYFETPNGKLFTQSFLPLDGEIKGTVYMSHGYGSDTSWMFQKICMSFSSWGYAVFAADLLGHGRSDGIRCYMGDMEKVAATSLAFFKHVRCSDPYKDLPAFLFGESMGGLVTLLMYFQSEPETWTGLMFSAPLFVIPEDMKPSKAHLFAYGLLFGLADTWAAMPDNKMVGKAIKDPEKLKIIASNPQRYTGKPRVGTMRELLRKTQYVQENFGKVTIPVFTAHGTADGVTCPTSSKLLYEKASSADKTLKIYEGMYHSLIQGEPDENAEIVLKDMREWIDEKVKKYGSKTA

SEQ ID NO: 2 - protein, 338 aa, Sorghum bicolor
MQADGDAPAPAPAVHFWGEHPATEAEFYAAHGAEGEPSYFTTPDAGARRLFTRAWRPRAPERPRALVFMVHGYGNDVSWTFQSTAVFLARSGFACFAADLPGHGRSHGLRAFVPDLDAAVADLLAFFRAVRAREEHAGLPCFLFGESMGGAICLLIHLRTRPEEWAGAVLVAPMCRISDRIRPPWPLPEILTFVARFAPTAAIVPTADLIEKSVKVPAKRIVAARNPVRYNGRPRLGTVVELLRATDELGKRLGEVSIPFLVVHGSADEVTDPEVSRALYAAAASKDKTIKIYDGMLHSLLFGEPDENIERVRGDILAWLNERCTPPATPWHRDIPVE

SEQ ID NO: 3 - protein, 353 aa, Sorghum bicolor
MMDVVYHEEYVRNPRGVQLFTCGWLPPASSSPPKALVFLCHGYGMECSDFMRACGIKLATAGYGVFGIDYEGHGKSMGARCYIQKFENLVADCDRFFKSICDMEEYRNKSRFLYGESMGGAVALLLHRKDPTFWDGAVLVAPMCKISEKVKPHPVVVTLLTQVEEIIPKWKIVPTKDVIDSAFKDPVKREKIRKNKLIYQDKPRLKTALELLRTSMDVEDSLSEVTMPFFILHGEADTVTDPEVSRALYERAASTDKTIKLYPGMWHGLTAGEPDENVELVFSDIVSWLDKRSRHWEQDERARTPPEPENKHRQAATTKITRVTSSSGGTESQRRGSCLCGLGGRPHQQQCRM

SEQ ID NO: 4 - protein, 348 aa, Sorghum bicolor
MEVEYHEEYVRNSRGVQLFTCGWLPVATSPKALVFLCHGYGMECSGFMRECGMRLAAAGYGVFGMDYEGHGKSMGARCYIRSFRRLVDDCSHFFKSICELEEYRGKSRFLYGESMGGAVALLLHRKDPAFWDGAVLVAPMCKISEKVKPHPVVITLLTQVEDVIPKWKIVPTKQDVIDAAFKDPVKREKIRRNKLIYQDKPRLKTALEMLRTSMYIEDSLSQVKLPFFVLHGEADTVTDPEVSRALYERAASADKTIKLYPGMWHGLTAGETDENVEAVFSDIVSWLNQRCRSWTMEDRFRKLVPAPAKFIHGDDAVDGKAQTQGRPRRRRPGLLCGLAGRTHHHAEM

SEQ ID NO: 5 - protein, 349 aa, Sorghum bicolor
MGRSSSSSGGGGADDGGEVLLDHEYKEEYVRNSRGMNLFACTWLPAGKRKTPKALVFLCHGYAVECGVTMRGTGERLARAGYAVYGLDYEGHGRSDGLQGYVPDFELLVQDCDEYFTSVVRSQSIEDKGCKLRRFLLGESMGGAVALLLDLRRPEFWTGAVLVAPMCKIADDMRPHPLVVNILRAMTSIVPTWKIVPSNDVIDAAYKTQEKRDEIRGNPYCYKDKPRLKTAYELLKVSLDLEQNLLHQVSLPFLIVHGGADKVTDPSVSELLYRSAASQDKTLKLYPGMWHALTSGESPDNIHTVFQDIIAWLDHRSSDDTDQQELLSEVEQKARHDEQHHQQQDGGNK

SEQ ID NO: 6 - DNA, 1017 bp, Sorghum bicolor
atgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccagggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtccatgggcggggccatctgcctgctcatccacctccgcacgcggccggaggagtgggcgggggcggtcctcgtcgcgcccatgtgcaggatctccgaccggatccgcccgccgtggccgctgccggagatcctcaccttcgtcgcgcgcttcgcgcccacggccgctatcgtgcccaccgccgacctcatcgagaagtccgtcaaggtgcccgccaagcgcatcgttgcagcccgcaaccctgtgcgctacaacggtcgccccaggctcggcaccgtcgtcgagctgttgcgtgccaccgacgagctgggcaagcgtctcggcgaggtcagcatcccgttccttgtcgtgcacggcagcgccgacgaggttactgacccggaagtcagccgcgccctgtacgccgccgccgccagcaaggacaagactatcaagatatacgacgggatgctccactccttgctatttggggaaccggacgagaacatcgagcgtgtccgcggcgacatcctggcctggctcaacgagagatgcacaccgccggcaactccctggcaccgtgacatacctgtcgaataa

SEQ ID NO: 7 - DNA, 999 bp, Arabidopsis thaliana
atgccgtcggaagcggagagctcagcgaattcagctccggcaactccgccaccaccaccgaatttctggggaaccatgccggaggaagagtactacacttcacaaggagtacgtaacagcaaatcatacttcgaaacaccaaacggcaagctcttcactcagagcttcttaccattagatggtgaaatcaaaggcactgtgtacatgtctcatggatacggatccgatacaagctggatgtttcagaagatctgtatgagtttctctagttggggttacgctgttttcgccgccgatcttctcggtcacggccgttccgatggtatccgctgctacatgggtgatatggagaaagttgcagcaacatcattggctttcttcaagcatgttcgttgtagtgatccatataaggatcttccggcttttctgtttggtgaatcgatgggaggtcttgtgacgcttttgatgtattttcaatcggaacctgagacttggaccggtttgatgttttcggctcctctctttgttatccctgaggatatgaaaccaagcaaggctcatctttttgcttatggtctcctctttggtttggctgatacgtgggctgcaatgccggataataagatggttgggaaggctatcaaggaccctgaaaagcttaagatcatcgcttctaacccgcaaagatatacagggaagcctagagtgggaacaatgagagagttactgaggaagactcaatacgttcaggagaatttcgggaaagttactattccggtgtttacggcgcacgggacagcggatggagtaacatgtcctacatcttcgaagctactatacgaaaaagcgtcaagcgctgataaaacgttgaagatctatgaagggatgtatcactcgctgattcaaggagagcctgacgagaacgctgagatagtcttgaaggatatgagagagtggatcgatgagaaggttaagaagtatggatctaaaaccgcttga

SEQ ID NO: 8 - DNA, 1062 bp, Sorghum bicolor
atgatggacgtggtctaccatgaggagtacgtgaggaacccgaggggcgtgcagctcttcacctgcggctggctgccgccggcgtcctcctccccacccaaagcgctcgtcttcctctgccatggctacgggatggagtgcagcgacttcatgagagcatgtgggatcaagttggccacagcagggtatggtgtgtttggcatcgattatgagggccacggcaagtccatgggcgccaggtgctacatccagaagtttgaaaacctcgtggctgactgcgacaggttcttcaagtccatctgtgatatggaggaatacagaaataaaagccggtttctgtacggcgagtcgatggggggagccgtggctctactactgcacaggaaggatccgaccttctgggatggtgcagttcttgtggcgccaatgtgcaagatttcagagaaagtgaaaccgcacccggtggtggtcaccctcctgactcaagtggaggagatcatcccaaagtggaagatcgtccccaccaaagacgtcatcgactcggcattcaaggaccccgtcaagcgcgaaaagatcaggaagaacaagctcatctaccaggacaagccccggctgaagacagctctggagctgctcaggaccagcatggatgtggaagatagcctgtcagaggtgacgatgccattcttcatcctgcacggcgaggccgacacggtgaccgacccggaggtcagccgcgccctctacgagcgcgccgccagcaccgacaagaccatcaagctgtaccctgggatgtggcacggcctcaccgccggcgagcctgacgagaacgtggagctggtgttctccgacatcgtctcgtggctcgacaagcgcagtcgccactgggagcaagacgagcgggcaaggactccaccggagcctgagaacaagcaccgccaggcggcgaccaccaaaatcacacgcgttaccagcagcagtggcggcaccgagagtcagcggcgcggcagctgcctctgcggactaggcggtcgaccacaccagcaacagtgtaggatgtag

SEQ ID NO: 9 - DNA, 1047 bp, Sorghum bicolor
atggaggtcgaataccacgaggagtacgtgcggaactcgagaggagtgcagctcttcacctgcggctggctgcccgtcgccacatcgcccaaggcgctcgtcttcctctgccacggctacggcatggagtgcagtggcttcatgagagaatgcggcatgcggctggcggcggccgggtacggcgtgttcgggatggactacgagggccacggcaagtccatgggcgcccgctgctacatccgcagcttccgccgcctcgtcgacgactgcagccatttcttcaagtccatctgcgagcttgaggagtaccggggcaagagccggttcctgtacggcgagtccatgggcggcgcggtggcgctgctgctgcacaggaaggaccccgccttctgggacggcgccgtgctcgtcgcgccgatgtgcaagatatcagagaaggtgaagccacaccctgtggtgatcacgctcctgacgcaggtggaggacgtgatcccaaagtggaagatcgtgcccaccaagcaggacgtcatcgacgccgccttcaaggaccccgtcaagcgtgaaaagatcaggagaaataagctgatttaccaggacaaaccgcggctcaagactgcgctagagatgctcaggaccagcatgtacatagaagacagcttgtcacaggtgaagctgccgttcttcgtcctgcacggcgaggccgacacggtgacggacccggaggtgagccgcgccctgtacgagcgcgcggcgagcgccgacaagaccatcaagctctacccggggatgtggcacggcctcaccgctggggagaccgacgagaacgtggaggccgtcttctccgacatcgtctcctggctcaaccagcgctgccggagctggaccatggaggaccgcttcaggaagctggtgccagcgccggccaagttcatccatggtgacgacgccgtcgacggcaaagcgcagacacaagggcgtcctcggcggcggcgtccgggcctcctctgcgggcttgccggccggacgcatcaccatgcagaaatgtga

SEQ ID NO: 10 - DNA, 1050 bp, Sorghum bicolor
atggggaggagcagcagcagcagcggcggcggcggggcagatgatggcggcgaggtgctcctggaccacgagtacaaagaggagtacgtgaggaactcgcgggggatgaacctgttcgcatgcacatggctgcctgcaggcaagaggaagactccaaaggcgctcgtcttcctctgccatggctacgccgtggagtgcggggtgacgatgcgcggcaccggcgagcgcctggcgcgtgcgggctacgccgtgtacggcctggactacgagggccacggccgctccgacgggctgcagggctacgtgccggatttcgagttgctggtccaggactgcgacgagtacttcacctccgtggtgcggtcgcagtcaattgaggacaagggctgcaagctgcgccggttcctgctcggtgagtccatgggcggcgccgtcgctctccttctagacctcaggcggcccgagttctggaccggcgccgtgctggtggcgcccatgtgcaagatcgccgacgacatgcgtccccacccgctggtggtgaacatcctgagggccatgacctccatcgtcccgacctggaagatcgtgcccagcaacgacgtgatcgacgccgcctacaagacgcaggagaagagggacgagatccggggcaacccttactgctacaaggacaagcccagactcaagaccgcgtacgaactgctcaaggtcagcttggacctcgagcaaaacctcctacaccaggtgtcgttgccgttcctgatcgtacacggcggagcagacaaggtgacggacccgtcggtgagcgagctgctgtaccgatcggcagcgagccaggacaagaccctgaagctctacccgggcatgtggcacgccctcacctccggcgagtcgcccgacaacatccacaccgttttccaagacatcatcgcctggctcgaccacagatcatccgacgacacggatcagcaggagctgctgtcggaggtggagcagaaggctaggcacgacgaacaacatcaccagcagcaggatggcggcaacaaatag

SEQ ID NO: 11 - DNA, 242 bp, Artificial Sequence
  Fragment
ccaaccaaccccaccacgccaacgtccgggaccaaactctgatccccaccatgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccagg

SEQ ID NO: 12 - DNA, 250 bp, Artificial Sequence
  fragment
gggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtc

SEQ ID NO: 13 - DNA, 193 bp, Artificial Sequence
  Fragment
atcgagcgtgtccgcggcgacatcctggcctggctcaacgagagatgcacaccgccggcaactccctggcaccgtgacatacctgtcgaataagcattccaggctgttcagattccgatgtatcgattacacaagaaaattggtttcatgtacaacgattcttatactatacgctatatacttggtcgtattt

SEQ ID NO: 14 - DNA, 612 bp, Artificial Sequence
  Recombinant construct
gagctcggcgcgccccaaccaaccccaccacgccaacgtccgggaccaaactctgatccccaccatgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccaggccgcgaagcaggcgaacccggaccgcgcgaggaagaccgccgtggactggaacgtccagctgacgtcgttgccgtagccgtggaccatgaagacgagcgccctgggccgctcgggcgcgcggggcctccacgcgcgcgtgaagagccgccgggcgcccgcgtcgggcgtggtgaagtaggagggctcgccctccgcgccgtgcgccgcgtagaactccgcctccgtggccgggtgctcgccccagaagtggacggccggcgccggcgccggcgcgtccccgtccgcctgcatggtggggatcagagtttggtcccggacgttggcgtggtggggttggttggatttaaatggtacc

SEQ ID NO: 15 - DNA, 612 bp, Artificial Sequence
  Recombinant construct
gagctcggcgcgcccctgggccgctcgggcgcgcggggcctccacgcgcgcgtgaagagccgccgggcgcccgcgtcgggcgtggtgaagtaggagggctcgccctccgcgccgtgcgccgcgtagaactccgcctccgtggccgggtgctcgccccagaagtggacggccggcgccggcgccggcgcgtccccgtccgcctgcatggtggggatcagagtttggtcccggacgttggcgtggtggggttggttggtgccccgtcgcaactggcagcagcagcgaccagcgactcccccaactcgccggccaccagtagttccctgcttccccatcccatccacacacaccgcacaccaaccaaccccaccacgccaacgtccgggaccaaactctgatccccaccatgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccaggatttaaatggtacc

SEQ ID NO: 16 - DNA, 628 bp, Artificial Sequence
  Recombinant DNA construct
gagctcggcgcgccgggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtcccggtcggagatcctgcacatgggcgcgacgaggaccgcccccgcccactcctccggccgcgtgcggaggtggatgagcaggcagatggccccgcccatggactccccgaagaggaagcagggcaggcccgcgtgctcctccctcgccctgacggcgcggaagaaggcgaggaggtcggcgacggcggcgtcgaggtcgggcacgaaggcgcggaggccgtgggagcggccgtggcccgggaggtcggccgcgaagcaggcgaacccggaccgcgcgaggaagaccgccgtggactggaacgtccagctgacgtcgttgccgtagccgtggaccatgaagacgagcgcccatttaaatggtacc

SEQ ID NO: 17 - DNA, 628 bp, Artificial Sequence
  Recombinant DNA construct
gagctcggcgcgccgactccccgaagaggaagcagggcaggcccgcgtgctcctccctcgccctgacggcgcggaagaaggcgaggaggtcggcgacggcggcgtcgaggtcgggcacgaaggcgcggaggccgtgggagcggccgtggcccgggaggtcggccgcgaagcaggcgaacccggaccgcgcgaggaagaccgccgtggactggaacgtccagctgacgtcgttgccgtagccgtggaccatgaagacgagcgccccacggcgcggagggcgagccctcctacttcaccacgcccgacgcgggcgcccggcggctcttcacgcgcgcgtggaggccccgcgcgcccgagcggcccagggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtcatttaaatggtacc

SEQ ID NO: 18 - DNA, 520 bp, Artificial Sequence
  Recombinant DNA construct
gagctcggcgcgccatcgagcgtgtccgcggcgacatcctggcctggctcaacgagagatgcacaccgccggcaactccctggcaccgtgacatacctgtcgaataagcattccaggctgttcagattccgatgtatcgattacacaagaaaattggtttcatgtacaacgattcttatactatacgctatatacttggtcgtattttattatcgaccccaagcatttgcagcattcttttacactgatcaggcaaccaacattttgtatatccaagccactaaacctgaccagacagtttatagtcaaatacgaccaagtatatagcgtatagtataagaatcgttgtacatgaaaccaattttcttgtgtaatcgatacatcggaatctgaacagcctggaatgcttattcgacaggtatgtcacggtgccagggagttgccggcggtgtgcatctctcgttgagccaggccaggatgtcgccgcggacacgctcgatatttaaatggtaccctcgat

SEQ ID NO: 19 - DNA, 514 bp, Artificial Sequence
  Recombinant DNA construct
gagctcggcgcgccaaatacgaccaagtatatagcgtatagtataagaatcgttgtacatgaaaccaattttcttgtgtaatcgatacatcggaatctgaacagcctggaatgcttattcgacaggtatgtcacggtgccagggagttgccggcggtgtgcatctctcgttgagccaggccaggatgtcgccgcggacacgctcgattcagccgcgccctgtacgccgccgccgccagcaaggacaagactatcaagatatacgacgggatgctccactccttgctatttggggaaccggacgagaaatcgagcgtgtccgcggcgacatcctggcctggctcaacgagagatgcacaccgccggcaactccctggcaccgtgacatacctgtcgaataagcattccaggctgttcagattccgatgtatcgattacacaagaaaattggtttcatgtacaacgattcttatactatacgctatatacttggtcgtatttatttaaatggtacc

SEQ ID NO: 20 - DNA, 2394 bp, Artificial Sequence
  Recombinant DNA construct
gaattcttaattaaggatccgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatcacccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagtattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcaggagctcgctaccttaagagaggtttaaacggtacccttttaagatgggatgtctttaatatgtagaacctcgtttttggttataattttcgttgcatgtctctcttctcttgtactattcacacttgttgtttgctgtatcttcttcttcagtttgctttgctacgattgtggtttttggagacattatagctcattaactgtttgtgagaccaaatgtgtcagaatccgctattacacacctagttgtcaacattcactacaaataatatggactttaacgtcggtttaaggcatccaataaaactgacgttatgtttctctttcctcgttttgtcgaccaaaaaaactgaccctaaatgtagatctttaattaaaagctt

SEQ ID NO: 21 - DNA, 2403 bp, Artificial Sequence
  Recombinant DNA construct
gaattcttaattaaggatccaaaattatggctaaaagtattgtttactgatttattatggaagaaaagcactactgactagcagaaaaagtacggcttataacacaaacgaacggaacctatgtactaactattaactagatcggtgctaaaatgtactccctccattcctaaataaattaaattctagagttatcttaaataaaacttttttaacgttttactgaatttatagaaagaaacacaaatatttatgacaccaaatgatcatattataaaaattattatggtgtatctcatgatactaatatagtgtcataaattttgacatttttattaaataaaataaaatttagtcaaattttaaaaagttggacttaaggcaaatctaaaagttgatttattcaggaatcagaggaagttaaaaaaaaatgattccagagctgttcttaaatttgttgcaaacacatggagggattgcttaaagatacatgggctcaggggatgctgcagtaccggtagcacctgccctgagctggcggacaactaaaatatttaagcaaaaaaaatgatggctacgattgtaaattgagcgtagttcagcaagtgaacccaatccaccatgttcaaatttttctatcttttttctagaatttaacaacgttgtgttttttaatgttaggagacatggtactatgatcaactgatcatttcgttaacctttttatgtacagcatcatcgagcatgcactggtccgagatataggcagcttaagcaccagttttatgtgcagccggataggtgatatgtccttgctaattaggctcctatttgtagctatagtattatctattcatacggccctatccattgctaagagcaagtataataagttatttttagccggttgcaagagtccacctaatcaaaaaagcagaccacgtaggagagatattagggcactcacaatgcaagactctatcacaaagtccaagacaattaattacatattatttatggtattttgctgatgtggcagcatatttattgaagaaagaggtagaaaaaaataagactccaagtcttatttagactctaagtccacattgttcgaggtaataaataactttagactctatgatagagtctgcattgtgagtgcccttatagagccggcgattcccatctcgcccgcctctagctcaagatacgagaaaaaaaaatttgtcctagacgtcttccagcccgctgtgagcgcgatgccgacgcttccatctcccgccgttccgctccctaattctgtgctctactcgatcattacctgacattaaatacttgtatttttattatagtacacctccaagctggctaaaccattttgatgtttaggttagtacatgttgatgtttaggttaggtgtaagtgatatgacaacttctctcaaccgtcagccggctaaaccattagccttgctctaactgggctttatttgttgctacagtactagtatctacaccttcggtcgtacccattttcacactctatgaaaacgctccgtttaatggaacttgttttctgcttaatctgccaaggctctcgttcatcaaaagaaaataaagcgagaatcaggtgatggagcgacatggttcttaaaatcatttttttcataaactaaaaatcgaaaggtttattggccctaataatgtcggtacacgagttaatgttccctgcatgggccaactatgaacgagaatagtataccacgtggacccgtgggccgcggcacgagccgttccacctacccgcaacgaaccgagcgatttcgccgtcccgcatccaaacgcccccagcagcccttcccctgccccagtgccccgtcgcaactggcagcagcagcgaccagcgactcccccaactcgccggccaccagtagttccctgcttccccatcccatccacacacaccgcacaccaaccaaccccaccacgccaacgtccgggaccaaactctgatccccaccggagctcgctaccttaagagaggtttaaacggtacccttttaagatgggatgtctttaatatgtagaacctcgtttttggttataattttcgttgcatgtctctcttctcttgtactattcacacttgttgtttgctgtatcttcttcttcagtttgctttgctacgattgtggtttttggagacattatagctcattaactgtttgtgagaccaaatgtgtcagaatccgctattacacacctagttgtcaacattcactacaaataatatggactttaacgtcggtttaaggcatccaataaaactgacgttatgtttctctttcctcgttttgtcgaccaaaaaaactgaccctaaatgtagatctttaattaaaagctt

SEQ ID NO: 22 - DNA, 20 bp, Artificial Sequence
  Synthetic oligonucleotide
tctaacggacaccaaccagc

SEQ ID NO: 23 - DNA, 20 bp, Artificial Sequence
  Synthetic oligonucleotide
ctgcatggtggggatcagag

SEQ ID NO: 24 - DNA, 20 bp, Artificial Sequence
  Synthetic oligonucleotide
tctaacggacaccaaccagc

SEQ ID NO: 25 - DNA, 20 bp, Artificial Sequence
  Synthetic oligonucleotide
cgggaccaaactctgatccc

SEQ ID NO: 26 - DNA, 20 bp, Artificial Sequence
  Synthetic oligonucleotide
tctaacggacaccaaccagc

SEQ ID NO: 27 - DNA, 20 bp, Artificial Sequence
  Synthetic oligonucleotide
ccgtggaccatgaagacgag

SEQ ID NO: 28 - DNA, 20 bp, Artificial Sequence
  Synthetic oligonucleotide
tctaacggacaccaaccagc

SEQ ID NO: 29 - DNA, 20 bp, Artificial Sequence
  Synthetic oligonucleotide
ctcgtcttcatggtccacgg

SEQ ID NO: 30 - DNA, 20 bp, Artificial Sequence
  Synthetic oligonucleotide
tctaacggacaccaaccagc

SEQ ID NO: 31 - DNA, 20 bp, Artificial Sequence
  Synthetic oligonucleotide
tgcatctctcgttgagccag

SEQ ID NO: 32 - DNA, 20 bp, Artificial Sequence
  Synthetic oligonucleotide
tctaacggacaccaaccagc

SEQ ID NO: 33 - DNA, 20 bp, Artificial Sequence
  Synthetic oligonucleotide
ccaggctgttcagattccga

SEQ ID NO: 34 - DNA, 20 bp, Artificial Sequence
  Synthetic oligonucleotide
ctgagctggcggacaactaa

SEQ ID NO: 35 - DNA, 20 bp, Artificial Sequence
  Synthetic oligonucleotide
gtggtgaagtaggagggctc

SEQ ID NO: 36 - DNA, 20 bp, Artificial Sequence
  Synthetic oligonucleotide
ctgagctggcggacaactaa

SEQ ID NO: 37 - DNA, 20 bp, Artificial Sequence
  Synthetic oligonucleotide
gagccctcctacttcaccac

SEQ ID NO: 38 - DNA, 20 bp, Artificial Sequence
  Synthetic oligonucleotide
ctgagctggcggacaactaa

SEQ ID NO: 39 - DNA, 20 bp, Artificial Sequence
  Synthetic oligonucleotide
ccgtggaccatgaagacgag

SEQ ID NO: 40 - DNA, 20 bp, Artificial Sequence
  Synthetic oligonucleotide
ctgagctggcggacaactaa

SEQ ID NO: 41 - DNA, 20 bp, Artificial Sequence
  Synthetic oligonucleotide
ctcgtcttcatggtccacgg

SEQ ID NO: 42 - DNA, 20 bp, Artificial Sequence
  Synthetic oligonucleotide
ctgagctggcggacaactaa

SEQ ID NO: 43 - DNA, 20 bp, Artificial Sequence
  Synthetic oligonucleotide
tgcatctctcgttgagccag

SEQ ID NO: 44 - DNA, 20 bp, Artificial Sequence
  Synthetic oligonucleotide
ctgagctggcggacaactaa

SEQ ID NO: 45 - DNA, 20 bp, Artificial Sequence
  Synthetic oligonucleotide
ccaggctgttcagattccga

SEQ ID NO: 46 - DNA, 20 bp, Artificial Sequence
  Synthetic oligonucleotide
ttcctcttcggggagtccat

SEQ ID NO: 47 - DNA, 20 bp, Artificial Sequence
  Synthetic oligonucleotide
tgcatctctcgttgagccag

SEQ ID NO: 48 - protein, 334 aa, Zea mays
MPADGEALAPAVHFWGEHPATEAEFYSAHGTEGESSYFTTPDAGARRLFTRAWRPRAPERPRALVFMVHGYGNDISWTFQSTAVFLARSGFACFAADLPGHGRSHGLRAFVPDLDAAVADLLAFFRAVRAREEHAGLPCFLFGESMGGAICLLIHLRTRPEEWAGAVLVAPMCRISDRIRPPWPLPEILTFVARFAPTAAIVPTADLIEKSVKVPAKRIVAARNPVRYNGRPRLGTVVELLRATDELAKRLGEVSIPFLVVHGSTDEVTDPEVSRALYAAAASKDKTIKIYDGMLHSLLFGEPDENIERVRGDILAWLNERCTAQATHRNIPVE

SEQ ID NO: 49 - DNA, 1497 bp, Zea mays
ccaccaaggcaccaacccgaaacgaatccagtgatttcccctcccgcatcgaaacgtcccccaagcagccctgcccggctgcccctgccgcgacgcaactggcaagcatccagcatagcagcgactcccccgctcgccggccagcggccaccagttccctttacatccacacacaacgcgcaccacaccacaccacccgacgccaacgtccgggaccaaactccgatccccaccactatgccggcggacggggaggcgctggcgccggccgttcacttctggggcgagcacccggccacggaggcggagttctactcggcgcacggcacggagggcgagtcctcctacttcaccacgcccgacgcgggcgcccggcggctcttcacgcgcgcgtggaggccccgcgcgcccgagcggcccagggcgctcgtgttcatggtccacggctacggcaacgacatcagctggacgttccagtccacggcggtcttcctcgcgcggtccgggttcgcctgcttcgcggccgacctcccgggccacggccgctcccacggcctccgcgccttcgtgcccgacctcgacgccgccgtcgctgacctcctcgccttcttccgcgccgtcagggcgagggaggagcacgcgggcctgccctgcttcctgttcggggagtccatgggcggggccatctgcctgctcatccacctccgcacacggccggaggagtgggcgggggcggtcctcgtcgctcccatgtgcaggatctccgaccggatccgcccgccgtggccgctgccggagattctcaccttcgtcgcgcgcttcgcgcccacggcggccatcgtgcccaccgccgacctcatcgagaagtccgtcaaggtgcccgccaagcgcatcgttgcagcgcgcaaccctgtgcgctacaacggccgtcccaggctcggcaccgtcgtcgagctgttgcgtgccaccgacgagctggccaagcgcctcggcgaagtcagcatcccgttccttgtcgtgcacggcagcaccgacgaggttaccgacccggaagtcagccgcgccctgtacgccgccgccgccagcaaggataagactatcaagatatacgacgggatgctccactccttgctatttggggaaccggacgagaacatcgagcgtgtccgtggggacatcctggcctggctcaatgagagatgcacagcccaggcaactcaccgtaacatacctgtcgaataagcattcggatgcatggatacacaagaaaaatgtttcatgtacaacgattgttatatatgctatactcagtatttgactgtaaactgttcggtcaggtttagtggcttggatatacaaaatgttggttgcctcatcagtgtaaaagaatgctgcaaatgcttgggatcgataatatcagctctcttcgggggctatggatggcaatacaaggcgttctctgccctgtacaagcttggcagaccgaattttatctcc

SEQ ID NO: 50 - protein, 335 aa, Setaria italica
MPADGDAPAPAVHFWGDHPATESDYYAAHGAEGEPSYFTTPDEGARRLFTRAWRPRAPARPKALVFMVHGYGNDISWTFQSTAVFLARSGFACFAADLPGHGRSHGLRAFVPDLDAAVADLLAFFRAVRAREEHAGLPCFLFGESMGGAICLLIHLRTPPEEWAGAVLVAPMCRISDRIRPPWPLPEILTFVARFAPTAAIVPTADLIEKSVKVPAKRVIAARNPVRYNGRPRLGTVVELLRATDELAKRLGEVTIPFLVVHGSADEVTDPEVSRALYEAAASKDKTIKIYDGMLHSLLFGELDENIERVRGDILAWLNEKCTLSTSLQRDITVE

SEQ ID NO: 51 - DNA, 1100 bp, Setaria italica
cgactcccccactcgccggccaccagtagttccccatccacaccgcatccccaccccacgccaccgtccggaaccaaaccctgatccccaccatgccggcggacggggacgcgccggcgccggccgtccacttctggggggaccacccggccacggagtccgactactacgccgcgcacggcgcggagggcgagccgtcctacttcaccacgcccgacgagggcgcccggcggctcttcacgcgcgcctggaggccccgcgcgccggcgcgccccaaggcgctcgtcttcatggtccacggctacggcaacgacatcagctggacgttccagtccacggcggtcttcctcgcgaggtccgggttcgcctgcttcgcggccgacctcccgggccacggccgctcccatggcctccgcgccttcgtgcccgacctcgacgccgccgtcgccgacctcctcgccttcttccgcgccgtcagggcgcgggaggagcacgcgggcctgccctgcttcctcttcggggagtccatgggcggcgccatctgcctgctcatccacctccgcacgccgcccgaggagtgggcgggggccgtcctcgtcgcgcccatgtgcaggatctcagaccggatccgcccgccgtggccgctgccggagatcctcaccttcgtcgcccggttcgcgcccaccgccgccatcgtgcccaccgccgacctcatcgagaagtccgtcaaggtgcccgccaagcgcgtcattgcggcgcgcaaccccgtgcgctacaacggccgccccaggctcggcaccgtcgtcgagctgctgcgcgccaccgacgagctggccaagcgcctcggcgaggtcaccatcccgttcctcgtcgtgcacggcagcgccgacgaggtcaccgaccccgaagtcagccgcgccctgtacgaggccgcagccagcaaggacaagaccatcaagatatacgacgggatgctccactccttgctcttcggggagctggacgagaacatcgagcgcgttcgtggcgacatcctcgcctggctcaacgagaaatgcacgctgtcaacttccttgcaacgtgacataactgttgaataa

SEQ ID NO: 52 - protein, 334 aa, Oryza sativa
MPDGERHEEAPDVNFWGEQPATEAEYYAAHGADGESSYFTPPGGRRLFTRAWRPRGDGAPRALVFMVHGYGNDISWTFQSTAVFLARSGFACFAADLPGHGRSHGLRAFVPDLDSAIADLLAFFRSVRRREEHAGLPCFLFGESMGGAICLLIHLRTPPEEWAGAVLVAPMCKISDRIRPPWPLPQILTFVARFAPTLAIVPTADLIEKSVKVPAKRLIAARNPMRYSGRPRLGTVVELLRATDELGARLGEVTVPFLVVHGSADEVTDPDISRALYDAAASKDKTIKIYDGMMHSMLFGEPDENIERVRADILAWLNERCTPREEGSFLTIQD

SEQ ID NO: 53 - DNA, 1336 bp, Oryza sativa
aaaaccgaaacgccgaacgaaacgaatcgtaaactcccctgctgctacgcaacgactccccaactctccggccaccaccaccaccacctgttccccatccgcacgccacgcaccggcccaaccgattccccaccatgccggacggcgagcggcatgaggaggccccggatgtgaacttctggggcgagcagccggcgacggaggctgagtactacgcggcgcacggcgcggatggcgagtcgtcctacttcaccccgccgggcgggcgccgcctcttcacgcgggcgtggcggccccgtggcgacggcgcgccgcgggcgctcgtgttcatggtgcacggctacggcaacgacatcagctggacgttccagtccacggccgtcttcctcgcccgctccggcttcgcctgcttcgccgccgacctccccggccatggccgctcccacggcctccgcgcgttcgtccccgacctcgattccgccatcgccgacctgctcgccttcttccgctccgtccggcggcgggaggagcacgccgggctgccgtgcttcctgttcggggagtccatgggcggggccatctgcctcctcatccacctccgcacgccgccggaggagtgggccggcgccgtgctggtggcgcccatgtgcaagatctccgaccggatccgcccgccatggccgctgccgcagatcctcaccttcgtcgcccgcttcgcgcccacgctcgccatcgtccccaccgccgacctcatcgagaagtccgtcaaggtgccggccaagcgcctcatcgccgcgcgcaaccccatgcgctatagcggccggccgaggctcggcaccgtcgtcgagctgctgcgcgccaccgacgagctcggcgcccgcctcggcgaagtcaccgtcccgttcctcgtcgtgcacggcagcgccgacgaggtgaccgacccggacatcagccgcgcgctgtacgacgccgccgccagcaaggacaagaccatcaagatatacgacgggatgatgcactccatgctcttcggggagcctgacgagaacatcgagcgcgtccgcgctgacattctcgcgtggctcaacgagagatgcacgccgagggaggagggcagcttcctgacaatacaagattagtatccaggattcactccactctattcagattattgtgaagtagcaaatgcacaaaaagaatgattaaatgtgcaaatttgcagtgattctatatataaatttgatgaacatttgcagtgattctatatataaatttgatgaactgctcagtcaggtttacatgatttatggtataaaatatgctaagtctcctgacc

SEQ ID NO: 54 - protein, 339 aa, Panicum virgatum
MAPPGDPPPATKYFWGDTPEPDEYYAAQGLRHAESYFQSPHGRLFTHAFHPLAGDVKGVVFMTHGYGSDSSWLFQTAAISYARWGYAVFCADLLGHGRSDGLRGYVGDMEAAAAASLAFFLSVRASAAYAALPAFLFGESMGGAATLLMYLRSPPSARWTGLVLSAPLLVIPDGMYPSRLRLFLYGLLFGLADTWAVLPDKRMVGKAIKDPDKLRLIASNPLGYRGAPRVGTMRELVRVTDLLRESLGEVAAPFLAVHGTDDGVTSPEGSRMLYERASSEDKELILYEGMYHSLIQGEPDENRDRVLADMRRWIDERVRRYGPAAAANGGGGKEEPPAP

SEQ ID NO: 55 - DNA, 1250 bp, Panicum virgatum
agagctcagaccatcttcccagcacactccggcgatggcgccgcccggggacccgccgccggcgaccaagtacttctggggcgacacccccgagcccgacgagtactacgccgcgcaggggctccggcacgccgagtcctacttccagtcccctcacggccgcctcttcacccacgccttccacccgctcgccggcgacgtcaagggcgtcgtcttcatgacccacggctacggttccgactcctcgtggctcttccagaccgccgccatcagctacgcgcgctgggggtacgccgtcttctgcgccgacctcctcggccacggccgctccgacggcctccgcgggtacgtcggcgacatggaggccgccgccgcggcgtccctcgctttcttcctctccgtgcgcgccagcgcggcgtacgccgcgctcccggcgttcctgttcggcgagtccatgggcggcgccgccacgctgctcatgtacctccgctccccgccgtccgcgcgctggacggggctcgtgctctcggcgccgctcctcgtcatccccgacggcatgtacccgtcccgcctccgcctcttcctgtacggcctcctcttcggcctcgccgacacctgggccgtgctcccggacaagaggatggtggggaaggcgatcaaggaccccgacaagctgcggcttatcgcgtccaacccgctcggctaccgcggcgcgccgcgggtgggcacgatgcgggagctggtccgcgtgacggatctgctgcgggagagcctcggggaggtggcggcgccgttcctcgccgtgcacgggacggacgacggcgtgacctcgccggaggggtccaggatgctgtacgagcgcgcgagcagcgaggacaaggagctcatcctgtacgaggggatgtaccactcgctcatccagggggagcccgacgagaaccgcgaccgcgtgctcgccgacatgcgcaggtggatcgacgagcgcgtgcgccgctacggccccgccgccgccgccaacgggggcggcggcaaggaggagccgccggcgccctgacggtgcggtgcagtgttggttgtcacttattcccatcacaactccattcctgtttcttgtttttcttttgggtaatcgctcattcgcttgtagttttacgaagatgatgggcgtcgagtgccatcgactgcaagaaatatctgaactataccttttgctttccttaaaaaaaaagagcttttgctttccttggacc

SEQ ID NO: 56 - DNA, 250 bp, Zea mays
gggcgctcgtgttcatggtccacggctacggcaacgacatcagctggacgttccagtccacggcggtcttcctcgcgcggtccgggttcgcctgcttcgcggccgacctcccgggccacggccgctcccacggcctccgcgccttcgtgcccgacctcgacgccgccgtcgctgacctcctcgccttcttccgcgccgtcagggcgagggaggagcacgcgggcctgccctgcttcctgttcggggagtc

SEQ ID NO: 57 - DNA, 250 bp, Setaria italica
ggcgctcgtcttcatggtccacggctacggcaacgacatcagctggacgttccagtccacggcggtcttcctcgcgaggtccgggttcgcctgcttcgcggccgacctcccgggccacggccgctcccatggcctccgcgccttcgtgcccgacctcgacgccgccgtcgccgacctcctcgccttcttccgcgccgtcagggcgcgggaggagcacgcgggcctgccctgcttcctcttcggggagtcc

SEQ ID NO: 58 - DNA, 147 bp, Oryza sativa
gcgcccatgtgcaagatctccgaccggatccgcccgccatggccgctgccgcagatcctcaccttcgtcgcccgcttcgcgcccacgctcgccatcgtccccaccgccgacctcatcgagaagtccgtcaaggtgccggccaagcgc

SEQ ID NO: 59 - DNA, 627 bp, Artificial Sequence
  Synthetic polynucleotide
gagctcggcgcgcgggcgctcgtgttcatggtccacggctacggcaacgacatcagctggacgttccagtccacggcggtcttcctcgcgcggtccgggttcgcctgcttcgcggccgacctcccgggccacggccgctcccacggcctccgcgccttcgtgcccgacctcgacgccgccgtcgctgacctcctcgccttcttccgcgccgtcagggcgagggaggagcacgcgggcctgccctgcttcctgttcggggagtcccggtcggagatcctgcacatgggagcgacgaggaccgcccccgcccactcctccggccgtgtgcggaggtggatgagcaggcagatggccccgcccatggactccccgaacaggaagcagggcaggcccgcgtgctcctccctcgccctgacggcgcggaagaaggcgaggaggtcagcgacggcggcgtcgaggtcgggcacgaaggcgcggaggccgtgggagcggccgtggcccgggaggtcggccgcgaagcaggcgaacccggaccgcgcgaggaagaccgccgtggactggaacgtccagctgatgtcgttgccgtagccgtggaccatgaacacgagcgcccatttaaatggtacc

SEQ ID NO: 60 - DNA, 627 bp, Artificial sequence
  Synthetic polynucleotide
gagctcggcgcgcggcgctcgtcttcatggtccacggctacggcaacgacatcagctggacgttccagtccacggcggtcttcctcgcgaggtccgggttcgcctgcttcgcggccgacctcccgggccacggccgctcccatggcctccgcgccttcgtgcccgacctcgacgccgccgtcgccgacctcctcgccttcttccgcgccgtcagggcgcgggaggagcacgcgggcctgccctgcttcctcttcggggagtcctccggtctgagatcctgcacatgggcgcgacgaggacggcccccgcccactcctcgggcggcgtgcggaggtggatgagcaggcagatggcgccgcccatggactccccgaagaggaagcagggcaggcccgcgtgctcctcccgcgccctgacggcgcggaagaaggcgaggaggtcggcgacggcggcgtcgaggtcgggcacgaaggcgcggaggccatgggagcggccgtggcccgggaggtcggccgcgaagcaggcgaacccggacctcgcgaggaagaccgccgtggactggaacgtccagctgatgtcgttgccgtagccgtggaccatgaagacgagcgccatttaaatggtacc

SEQ ID NO: 61 - DNA, 421 bp, Artificial sequence
  Synthetic polynucleotide
gagctcggcgcgcgcgcccatgtgcaagatctccgaccggatccgcccgccatggccgctgccgcagatcctcaccttcgtcgcccgcttcgcgcccacgctcgccatcgtccccaccgccgacctcatcgagaagtccgtcaaggtgccggccaagcgccgaggcgggcgccgagctcgtcggtggcgcgcagcagctcgacgacggtgccgagcctcggccggccgctatagcgcatggggttgcgcgcggcgatgaggcgcttggccggcaccttgacggacttctcgatgaggtcggcggtggggacgatggcgagcgtgggcgcgaagcgggcgacgaaggtgaggatctgcggcagcggccatggcgggcggatccggtcggagatcttgcacatgggcgcatttaaatggtacc

SEQ ID NO: 62 - DNA, 2020 bp, Sorghum bicolor
gaattcttaattaaggatccaaaattatggctaaaagtattgtttactgatttattatggaagaaaagcactactgactagcagaaaaagtacggcttataacacaaacgaacggaacctatgtactaactattaactagatcggtgctaaaatgtactccctccattcctaaataaattaaattctagagttatcttaaataaaacttttttaacgttttactgaatttatagaaagaaacacaaatatttatgacaccaaatgatcatattataaaaattattatggtgtatctcatgatactaatatagtgtcataaattttgacatttttattaaataaaataaaatttagtcaaattttaaaaagttggacttaaggcaaatctaaaagttgatttattcaggaatcagaggaagttaaaaaaaaatgattccagagctgttcttaaatttgttgcaaacacatggagggattgcttaaagatacatgggctcaggggatgctgcagtaccggtagcacctgccctgagctggcggacaactaaaatatttaagcaaaaaaaatgatggctacgattgtaaattgagcgtagttcagcaagtgaacccaatccaccatgttcaaatttttctatcttttttctagaatttaacaacgttgtgttttttaatgttaggagacatggtactatgatcaactgatcatttcgttaacctttttatgtacagcatcatcgagcatgcactggtccgagatataggcagcttaagcaccagttttatgtgcagccggataggtgatatgtccttgctaattaggctcctatttgtagctatagtattatctattcatacggccctatccattgctaagagcaagtataataagttatttttagccggttgcaagagtccacctaatcaaaaaagcagaccacgtaggagagatattagggcactcacaatgcaagactctatcacaaagtccaagacaattaattacatattatttatggtattttgctgatgtggcagcatatttattgaagaaagaggtagaaaaaaataagactccaagtcttatttagactctaagtccacattgttcgaggtaataaataactttagactctatgatagagtctgcattgtgagtgcccttatagagccggcgattcccatctcgcccgcctctagctcaagatacgagaaaaaaaaatttgtcctagacgtcttccagcccgctgtgagcgcgatgccgacgcttccatctcccgccgttccgctccctaattctgtgctctactcgatcattacctgacattaaatacttgtatttttattatagtacacctccaagctggctaaaccattttgatgtttaggttagtacatgttgatgtttaggttaggtgtaagtgatatgacaacttctctcaaccgtcagccggctaaaccattagccttgctctaactgggctttatttgttgctacagtactagtatctacaccttcggtcgtacccattttcacactctatgaaaacgctccgtttaatggaacttgttttctgcttaatctgccaaggctctcgttcatcaaaagaaaataaagcgagaatcaggtgatggagcgacatggttcttaaaatcatttttttcataaactaaaaatcgaaaggtttattggccctaataatgtcggtacacgagttaatgttccctgcatgggccaactatgaacgagaatagtataccacgtggacccgtgggccgcggcacgagccgttccacctacccgcaacgaaccgagcgatttcgccgtcccgcatccaaacgcccccagcagcccttcccctgccccagtgccccgtcgcaactggcagcagcagcgaccagcgactcccccaactcgccggccaccagtagttccctgcttccccatcccatccacacacaccgcacaccaaccaaccccaccacgccaacgtccgggaccaaactctgatccccacc

SEQ ID NO: 63 - DNA, 695 bp, Sorghum bicolor
gcattccaggctgttcagattccgatgtatcgattacacaagaaaattggtttcatgtacaacgattcttatactatacgctatatacttggtcgtatttgactataaactgtctggtcaggtttagtggcttggatatacaaaatgttggttgcctgatcagtgtaaaagaatgctgcaaatgcttggggtcgataatatcagctctcttcgggggctattgatggcagcacaaggcgttccctgccttgtacaagcttggcagaacgaattttatccccggtcttaatctgcgatagaacatctcttccatccgtggtatacctgcaattgtttggatatacgcataacatttcttacagcgttcttatccacaatggaatagatcgattttgcaactcaatgtttacataatgaaatcagtcacgacttacccgaaaactgaaaactgtccctcatcaaacgatattcctcctaagccagactacagaaaagaaagagaaacatgttaactcacatatctatacagaaattcatgcttcttcagattattacaggctggagaagcaacttgttacttgttatattagtacattgggcattcatattctttgtatgactgacctggcagagtctggtctgttatctgaatacttatattcatctttatgtttaaagaaaagcaaatatggttt

SEQ ID NO: 64 - protein, 365 aa, Sorghum bicolor
MGSLASERKVVGWAARDATGHLSPYTYTLRNTGPEDVVVKVLYCGICHTDIHQAKNHLGASKYPMVPGHEVVGEVVEVGPEVSKYGVGDVVGVGVIVGCCRECSPCKANVEQYCNKKIWSYNDVYTDGRPTQGGFASTMVVDQKFVVKIPAGLAPEQAAPLLCAGVTVYSPLKAFGLTAPGLRGGIVGLGGVGHMGVKVAKAMGHHVTVISSSSKKRAEAMDHLGADAYLVSTDAAAMAAAADSLDYIIDTVPVHHPLEPYLSLLRLDGKHVLLGVIGEPLSFVSPMVMLGRKAITGSFIGSIDETAEVLQFCVDKGLTSQIEVVKMGYVNEALERLERNDVRYRFVVDVAGSNVEEDAADAPSN

SEQ ID NO: 65 - DNA, 1457 bp, Sorghum bicolor
gatcgcccaccctctcggcctctccaggccgccgccggctccgtcgtcgtgttccccgacgcccgtagcgttcgaccgcggccagtcccagtccaagaggagaatggggagcctggcgtccgagaggaaggtggtcggctgggccgccagggacgccaccggacacctctccccctacacctacaccctcaggaacacaggccctgaagatgtggtggtgaaggtgctctactgtggaatctgccacacggacatccaccaggccaagaaccacctcggggcttcaaagtaccctatggtccctgggcacgaggtggtcggtgaggtggtggaggtcgggcccgaggtgagcaagtatggcgtcggcgacgtggtaggcgtcggggtgatcgtcgggtgctgccgcgagtgcagcccctgcaaggccaacgttgagcagtactgcaacaagaagatctggtcctacaacgatgtctacactgacggccggcccacgcagggcggcttcgcctccaccatggtcgtcgaccagaagtttgtggtgaagatcccggcgggtctggcgccggagcaagcggcgccgctgctgtgcgcgggcgtgacggtgtacagcccgctaaaggcctttgggctgacggccccgggcctccgcggtggcatcgtgggcctgggcggcgtgggccacatgggcgtgaaggtggcgaaggccatgggccaccacgtgacggtgatcagctcgtcgtccaagaagcgcgcggaggcgatggaccacctgggcgcggacgcgtacctggtgagcacggacgcggcggccatggcggcggccgccgactcgctggactacatcatcgacacggtgcccgtgcaccacccgctggagccctacctgtcgctgctgaggctggacggcaagcacgtgctgctgggcgtcatcggcgagcccctcagcttcgtgtccccgatggtgatgctggggcggaaggccatcacggggagcttcatcggcagcatcgacgagaccgccgaggtgctccagttctgcgtcgacaaggggctcacctcccagatcgaggtggtcaagatggggtacgtgaacgaggcgctggagcggctcgagcgcaacgacgtccgctaccgcttcgtcgtcgacgtcgccggcagcaacgtcgaggaggatgccgctgatgcgccgagcaactgacggcgtgcaacgttcgttcggggctcgaggctgcctgcgcttctgcttcctttagtaattgtgggctttgtgcgttcttgccgtgttctgttctggttctgggctttcagatgagttgaaggatggtctgtttaaatggcatcagactgaataactatatgttgtagtagtacgtgttatactcggagtacgccacgatatggtgtggtgtcagtgtcaccagcattctggatttgcagtttacccaaaaaaaaaa

SEQ ID NO: 66 - DNA, 4870 bp, Sorhum bicolor
gttgttggaccatttataatttttctccagtagccaccgcagaagatcctgctggcaggtggcctgccggttgccggactgccacttttgcacagcgccgatcgagctcggctctccgactgcccctatatagcgcgcactccgctcacgcatttttttcctaccaaaaagacaggcgcactagttgtcgcgcggctttctttcccgaaggctgagccgggctcgtccgtctccatcgcccaccctctcggcctctccaggccgccgccggctccgtcgtcgtgttccccgacgcccgtagcgttcgaccgcggccagtcccagtccaagaggagaatggggagcctggcgtccgagaggaaggtggtcggctgggccgccagggacgccaccggacacctctccccctacacctacaccctcaggtacgccgctccgccgccgccgccgccactctagatcgctcgtgttcgtcttctcacttttcctacccctagtcccctcccccttcatgtccgtccgactgtgtctcctgctccttgtgcaaacacgaaaatagatccaggagaggatgagggacggtttggcttgtgcggcgccttcttcagtgattgtccgagatcgaccaggaacaggaagaacagtaaaatctgagtcatgattgtgatgattttttttttaaaaaaaaaaacaggatatatttccgatccacttccacgattaggccggtgcacgtatctaatcgccggcaggttttaatttgggaaggatgctatacgtatgcatattctgatccatatactataactgatacgtttacggttatcatttaccgagtattccttctcttgatttctgtaagatgttccttatgttatatgctgtggtcgtatctttttcctcacacatactgtagtatactagtacaccttagtaggagcactactccacaacaaacgcatgcatgcgcatgcgcgcggcagcatgcgcatgataggtcttcaactccaggtccaactctagtgccgccgcacatgcatgtatggatgccacggttgaggatatattttgcttcaatattaatatttgtgccctgcacctgcactgcacgtgagtttgacgacgtttcgtacagacccagtagccaacgtgttgtgtggagtagcttgtcgtactggcaggtacaataccagcaaacctaaaatatggatacgggtgatgacaccgtacctacagctacctaccacctggtagctgtttgcaacactggcctggcgcgcgcacaccataattcttaaattttttttgtttggttattgtagcattttgtttgtatttgataattattgttaatcatggattaactaagctcaaagaattcatctagcaaatgacagttaaactgtaccattagttattatttttgtttatatttaatacttcattatgtggcgtaagattcgatgtgatgaagaatcttaaaaagttttttggattttggggtaaactaaacaagaactagttggcgaaaaaatttgggtttggctattatagcacttttgtttaatttgtatttgacaattattatcccattaaagactagctaggctcaaaagattcgtctcgcaaattaaatgcaacctgtgcaattagttattttttaatctatatttaatgctccatgtatgtgtccaaagatttgatatgacggaaaattttgaaaaaatagaaaatttttggaactaaacagcctttataagtgatattattccgatcaggctggaggaaattgaacagccatgggtttgtttactcatatataagtgatcgatactgttgattattccgatcaggctggaggaaattgaacagcactacataaacccttggctttcggttcattaagtagtagtagtcttaatagtagtagtggtcactaggttatgtggtgcagtaatttgaaagcatccatccatcgcctgcatatacttttattattgcttcgagagaagactcttgcactgctttctcatgtcatcaactactagtgtacgatgatactatctagctaactgtggcggttcttgcatatttctatatgctgctggtccttctgcaagaataaactaattaacactggtctctttttatatgggatgtgctgtgggtgacaacaacaaaaacaggaacacaggccctgaagatgtggtggtgaaggtgctctactgtggaatctgccacacggacatccaccaggccaagaaccacctcggggcttcaaagtaccctatggtccctgggtgagcacaaacaaaccccctagctagcgattttatttttcagcacctttgggatcgagtaatactctgtatatggtttacgataaactgaattttccagtgttctattattcaaactgtctgaaaagtataaatgaataggacacatatatagcgacatgccgtttccgcattttgatgagaaaactacacatgcagacaaatttaggtatatctatctgattgacctgcatagactggtagataggtcagtgcacatttggtaactacaaacgtcagcatctcagtccgtagctattcttagatttacaggtggcacataccacactaaaactctttgttacgtagttggttgccaattactgtcattccatcagtttaccaaattatttgaagcacaagagtttgttgcgtctaagatgttcttttcatgatagctaaagagctgcagaaatgagtagtaaagcaaaccccaccggccggcctatataccttttttctgacatgtttgcgagggggaaaaaaattaaataaacataaacttttcctgacagcacaaccactccactactgcgaactgataatgtgcacactagctatcatgggttggtttttgctaatgtcgtgtgtctgaaacttttgcaggcacgaggtggtcggtgaggtggtggaggtcgggcccgaggtgagcaagtacggcgtcggcgacgtggtaggcgtcggggtgatcgtcgggtgctgccgcgagtgcagcccctgcaaggccaacgttgagcagtactgcaacaagaagatctggtcctacaacgatgtctacactgacggccggcccacgcagggcggcttcgcctccaccatggtcgtcgaccagaagtgagtttcttgaaactgaaaactaatcatcaggttcattcagcgttatcttgcctgcagtgttctagctagagataatttcttgttttttttttttaaaaaaagttggtctgaagtctgaactaagcaagaaatagttgagcttcagtttgaacttttgtggaagtggatggtgatgtccaatccttctagaaaaggtggaggggagagtatatgggtatgggaaaaaatttatcattgagagagtccatcatcgtccagctgcaagtcagcgtatggatgccttgtggtgaccaggcaagagtgtgatgtgaaaagtacgacgtggtgtgctttactggctcatctttgtcaagttgaaccataaccacagaagccgaatcctcacctactactcactactcatgtctgaagattggtcatccaaaccatcactggttgttgggagaaatggggataactttctccatcgtttgattccaaacttgcctgcgactttagtgtactgtctttttcagtcagtgggcaaatcacactacctaatccaacaactctttgagatagcgattgcttgtttttttttaaaaaaaaaatgggatatatgtgtgaattatgatagaacagtaactcctgaagctattttatttggtgctagttaaatactatccaacaactctttgagatagcgattgcttgttgataattaatgcattttgtttcaggtttgtggtgaagatcccggcgggtctggcgccggagcaagcggcgccgctgctgtgcgcgggcgtaacggtgtacagcccgctaaaggcctttgggctgacggccccgggcctccgcggtggcatcgtgggcctgggcggcgtgggccacatgggcgtgaaggtggcgaaggccatgggccaccacgtgacggtgatcagctcgtcgtccaagaagcgcgcggaggcgatggaccacctgggcgcggacgcgtacctggtgagcacggacgcggcggccatggcggcggccgccgactcgctggactacatcatcgacacggtgcccgtgcaccacccgctggagccctacctgtcgctgctgaggctggacggcaagcacgtgctgctgggcgtcatcggcgagcccctcagcttcgtgtccccgatggtgatgctggggcggaaggccatcacggggagcttcatcggcagcatcgacgagaccgccgaggtgctccagttctgcgtcgacaaggggctcacctcccagatcgaggtggtcaagatggggtacgtgaacgaggcgctggagcggctcgagcgcaacgacgtccgctaccgcttcgtcgtcgacgtcgccggcagcaacgtcgaggaggatgccgctgatgcgccgagcaactgacggcgtgcaacgttcgttcggggctcgaggctgcctgcgcttctgcttcctttagtaattgtgggctttgtgcgttcttgccgtgttctgttctggttctgggctttcagatgagttgaaggatggtctgtttaaatggcatcagactgaataactatatgttgtagtagtacgtgttatactcggagtacgccacgatatggtgtggtgtcagtgtcaccagcattctggatttgcagtttacccaaatgtttctggtgctgcgtctcctacactgggctaacctttttcagacgtatgcccaaatg

SEQ ID NO: 67 - DNA, 3000 bp, Sorghum bicolor
ttaattgacgtatttggtctttttgttcattacaatgttgaatgttcaatacaaaaagttctcgttgctaattaattagaaaacagcacgttattaattatataaaagaaataaaacaaataaaactgcaggaaccgtagacttcgtgcatgaaaagattaatgctagcatagaaaaagactataactaccctaatctagctagagtcaatatgtatgaaacactctggattagggtgccttaaccaacttatatatgcttcgaagtgagtctgaattccggatagctaattagttattagaattataggtcagtcttagtaaaagtttcattagggtttcatttgcattgtcacataagcgcgcacttttgatgatgtgacaacgtttttaaaaagagagggaagatgtaagttttacagggatgaaactcttttagtacgattatcaacactttattagtcatgaaatgaaagatctatatctacaaaaccatagaataaattttttcattgagatgatgtttcttacatgttttattttattctatatgacatgatattcttgaaaaataacgttacaaaactctctattaagacttaccttagttattgtttgaatcctccagctagctagtagttaattgcatttagatagagatagagagagccagctatttagctgagatatttggatggaagcagccaacagtaattagctgtgcagtggagtattttagctagctgaagggaggctttaatttgggtttgttcgaaaggtgacgtggtcctgacgtcagatcctgcgggccccactcacctaccacgcccaacgacccccggcatccctttcacgtttgtcatcctcctcgcggcttatcaatatcaactgcctcttcgcggcacgtcacttttctcccatgcatcagccagctcctcgtgcgcccaatctctacttcatttgctcctgatttgctcccatgcagaatctacggacaaatcaacccaccactggaaattaaaacgtacgattctgattgccgaagaaacaagcacctattgcttctccctccgtagcatggaaagagtattcgatatttttttcttttagaacatagagttttgtaactcttaaaagagtattgagaggaataaagaatgtcagctttaagacttttcaataatccgctcttaaaatatagaaacaattttacatcatgattcatatactaattctatctcttctctcttgtatatttaatttacctcagtaactttttcctactctttgttttcttcacgcccttccacctttagattagccgacccatgcacatcaaaaagaaaatacgcatgacttgaagtctgcggaactttacacgcaaataggagttttttctcccaagtgccaaaagattgggagatggattttttttttcattctttccaagaatcatgaattgaaaaagattattgggtacttttggagatgctcattctcttgtttataaataatatagtagttaatgttattctaaacggtaaatggattaacgtttaaacactcttgaaatggtttaaataaaatgtttatatggtattaccaaaatatgcatatctgttcatgtaaaaaagcttaatatgctaacaaagatatataagtgtatattctaagaaaattagttggctgccaacaatatggtggataggatcctcataccggttaaattattaattaaatgtctatttatcatgtctaatcatgtttatccctttcgcgttgtcttcctcctcacggcttatcgatatcaactcagcttcttcgtggcacatcactttaatttcctcccatcagtgggttcctccatctctacttcattagctcccatgcagaattatactaataacaaatcaatccacccgccgctggaaaatgtgcgaagaaacagcacctactgctccctccctagcacggaatggatgatgtcaactctctcttgtctataatagtagttatcagtcttattctaaacggtaaagggattaacgtgtagacaccgtttaaatggcctaaacaattctttatatattaccaaaatatgcatgtctattcatgtaaaaaagtttaatatggtaaaaaagatatataaatatgtatgtaaaagtgcatattctaagaaaaaaaatatgtatgtagactcaaatatttttttttacatttcctttcttttatttagtgcggaacgaatagtttcagtcttgcagacatgtttgaattcaataatttcttgaaagaacatcactgatgaaacccatataagcagcaggcacactctccttgttatcaaacttattccaatgaaattacgaatcaccaatagcttagtagcagcagccatgcttaacatgaagattctacaatggcaactgatacgcccaggtctgcaatattaaagatttagtttggttttccttaactcatgtcaagtagcactattaaatcttcaggattatgtacatcgttcccatcaaattatctaagaaaatgatgtcacggtccatcgtatatactatggaatacctttaaatatttcatgaaacttgatttcatcttattagaaatagtttttattttgttttctttctttcctctatatagtggtgagcaatgcaaatccgccgcaacacgcgagagagtattcatctatttctacagactattaacatcatgtttagaacatgagatttttccttttattttctttccctaccttattcctgtgaaattaaacgaaaattctatgaaattcctttgcaaaccctacaaaaaattcctacgtacattgcaaacatgtagctccaaatgatttgtccaatttgtcagtacatacagagcttggagctgcggtgttttcttggctgacctacatatggagccacgctcatgctgacctatcatccggggggctgtgtacgatttgccacttgccagtgggatcacg

SEQ ID NO: 68 - DNA, 23 bp, Sorghum bicolor
  misc_feature (21)..(21): n is a, c, g, or t
ggcgcggacgcgtacctggtnag

SEQ ID NO: 69 - DNA, 23 bp, Sorghum bicolor
  misc_feature (21)..(21): n is a, c, g, or t
aacggtgtacagcccgctaangg

SEQ ID NO: 70 - DNA, 23 bp, Sorghum bicolor
  misc_feature (21)..(21): n is a, c, g, or t
taacggtgtacagcccgctanag

SEQ ID NO: 71 - DNA, 23 bp, Sorghum bicolor
  misc_feature (21)..(21): n is a, c, g, or t
gcccggggccgtcagcccaangg

SEQ ID NO: 72 - DNA, 23 bp, Sorghum bicolor
  misc_feature (21)..(21): n is a, c, g, or t
gccatcacggggagcttcatngg

SEQ ID NO: 73 - DNA, 23 bp, Sorghum bicolor
  misc_feature (21)..(21): n is a, c, g, or t
gcgacaggtagggctccagcngg

SEQ ID NO: 74 - DNA, 23 bp, Sorghum bicolor
  misc_feature (21)..(21): n is a, c, g, or t
agcgacaggtagggctccagngg

SEQ ID NO: 75 - DNA, 23 bp, Sorghum bicolor
  misc_feature (21)..(21): n is a, c, g, or t
gatcccggcgggtctggcgcngg

SEQ ID NO: 76 - DNA, 23 bp, Sorghum bicolor
  misc_feature (21)..(21): n is a, c, g, or t
ccgcggaggcccggggccgtnag

SEQ ID NO: 77 - DNA, 23 bp, Sorghum bicolor
  misc_feature (21)..(21): n is a, c, g, or t
ccgcggaggcccggggccgtnag

SEQ ID NO: 78 - DNA, 23 bp, Sorghum bicolor
  misc_feature (21)..(21): n is a, c, g, or t
accaggtacgcgtccgcgccnag

SEQ ID NO: 79 - DNA, 23 bp, Sorghum bicolor
  misc_feature (21)..(21): n is a, c, g, or t
accaggtacgcgtccgcgccnag

SEQ ID NO: 80 - DNA, 667 bp, Sorghum bicolor
gtttgtggtgaagatcccggcgggtctggcgccggagcaagcggcgccgctgctgtgcgcgggcgtaacggtgtacagcccgctaaaggcctttgggctgacggccccgggcctccgcggtggcatcgtgggcctgggcggcgtgggccacatgggcgtgaaggtggcgaaggccatgggccaccacgtgacggtgatcagctcgtcgtccaagaagcgcgcggaggcgatggaccacctgggcgcggacgcgtacctggtgagcacggacgcggcggccatggcggcggccgccgactcgctggactacatcatcgacacggtgcccgtgcaccacccgctggagccctacctgtcgctgctgaggctggacggcaagcacgtgctgctgggcgtcatcggcgagcccctcagcttcgtgtccccgatggtgatgctggggcggaaggccatcacggggagcttcatcggcagcatcgacgagaccgccgaggtgctccagttctgcgtcgacaaggggctcacctcccagatcgaggtggtcaagatggggtacgtgaacgaggcgctggagcggctcgagcgcaacgacgtccgctaccgcttcgtcgtcgacgtcgccggcagcaacgtcgaggaggatgccgctgatgcgccgagcaactga

SEQ ID NO: 81 - DNA, 23 bp, Sorghum bicolor
gccatcacggggagcttcatcgg

SEQ ID NO: 82 - DNA, 23 bp, Sorghum bicolor
gccatcacggggagcttcatcgg

SEQ ID NO: 83 - DNA, 326 bp, Sorghum bicolor
gtttgtggtgaagatcccggcgggtctggcgccggagcaagcggcgccgctgctgtgcgcgggcgtaacccagggaccatagggtactttgaagccccgaggtggttcttggcctggtggatgtccgtgtggcagattccacagtagagcaccttcaccaccacatcttcagggcctgtgttcctgagggtgtaggtgtagggggagaggtgtccggtggcgtccctggcggcccagccgaccaccttcctctcggacgccaggctccccatcggcagcatcgacgagaccgccgaggtgctccagttctgcgtcgacaaggggct

SEQ ID NO: 84 - DNA, 1509 bp, Sorghum bicolor
ggtgatggagcgacatggttcttaaaatcatttttttcataaactaaaaatcgaaaggtttattggccctaataatgtcggtacacgagttaatgttccctgcatgggccaactatgaacgagaatagtataccacgtggacccgtgggccgcggcacgagccgttccacctacccgcaacgaaccgagcgatttcgccgtcccgcatccaaacgcccccagcagcccttcccctgccccagtgccccgtcgcaactggcagcagcagcgaccagcgactcccccaactcgccggccaccagtagttccctgcttccccatcccatccacacacaccgcacaccaaccaaccccaccacgccaacgtccgggaccaaactctgatccccaccatgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccagggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtccatgggcggggccatctgcctgctcatccacctccgcacgcggccggaggagtgggcgggggcggtcctcgtcgcgcccatgtgcaggatctccgaccggatccgcccgccgtggccgctgccggagatcctcaccttcgtcgcgcgcttcgcgcccacggccgctatcgtgcccaccgccgacctcatcgagaagtccgtcaaggtgcccgccaagcgcatcgttgcagcccgcaaccctgtgcgctacaacggtcgccccaggctcggcaccgtcgtcgagctgttgcgtgccaccgacgagctgggcaagcgtctcggcgaggtcagcatcccgttccttgtcgtgcacggcagcgccgacgaggttactgacccggaagtcagccgcgccctgtacgccgccgccgccagcaaggacaagactatcaagatatacgacgggatgctccactccttgctatttggggaaccggacgagaacatcgagcgtgtccgcggcgacatcctggcctggctcaacgagagatgcacaccgccggcaactccctggcaccgtgacatacctgtcgaataagcattccaggctgttcagattccgatgtatcgattacacaagaaaattggtttcatgtacaacgattcttatactatacgctatatacttggtcgtattt

SEQ ID NO: 85 - DNA, 2000 bp, Sorghum bicolor
aaaattatggctaaaagtattgtttactgatttattatggaagaaaagcactactgactagcagaaaaagtacggcttataacacaaacgaacggaacctatgtactaactattaactagatcggtgctaaaatgtactccctccattcctaaataaattaaattctagagttatcttaaataaaacttttttaacgttttactgaatttatagaaagaaacacaaatatttatgacaccaaatgatcatattataaaaattattatggtgtatctcatgatactaatatagtgtcataaattttgacatttttattaaataaaataaaatttagtcaaattttaaaaagttggacttaaggcaaatctaaaagttgatttattcaggaatcagaggaagttaaaaaaaaatgattccagagctgttcttaaatttgttgcaaacacatggagggattgcttaaagatacatgggctcaggggatgctgcagtaccggtagcacctgccctgagctggcggacaactaaaatatttaagcaaaaaaaatgatggctacgattgtaaattgagcgtagttcagcaagtgaacccaatccaccatgttcaaatttttctatcttttttctagaatttaacaacgttgtgttttttaatgttaggagacatggtactatgatcaactgatcatttcgttaacctttttatgtacagcatcatcgagcatgcactggtccgagatataggcagcttaagcaccagttttatgtgcagccggataggtgatatgtccttgctaattaggctcctatttgtagctatagtattatctattcatacggccctatccattgctaagagcaagtataataagttatttttagccggttgcaagagtccacctaatcaaaaaagcagaccacgtaggagagatattagggcactcacaatgcaagactctatcacaaagtccaagacaattaattacatattatttatggtattttgctgatgtggcagcatatttattgaagaaagaggtagaaaaaaataagactccaagtcttatttagactctaagtccacattgttcgaggtaataaataactttagactctatgatagagtctgcattgtgagtgcccttatagagccggcgattcccatctcgcccgcctctagctcaagatacgagaaaaaaaaatttgtcctagacgtcttccagcccgctgtgagcgcgatgccgacgcttccatctcccgccgttccgctccctaattctgtgctctactcgatcattacctgacattaaatacttgtatttttattatagtacacctccaagctggctaaaccattttgatgtttaggttagtacatgttgatgtttaggttaggtgtaagtgatatgacaacttctctcaaccgtcagccggctaaaccattagccttgctctaactgggctttatttgttgctacagtactagtatctacaccttcggtcgtacccattttcacactctatgaaaacgctccgtttaatggaacttgttttctgcttaatctgccaaggctctcgttcatcaaaagaaaataaagcgagaatcaggtgatggagcgacatggttcttaaaatcatttttttcataaactaaaaatcgaaaggtttattggccctaataatgtcggtacacgagttaatgttccctgcatgggccaactatgaacgagaatagtataccacgtggacccgtgggccgcggcacgagccgttccacctacccgcaacgaaccgagcgatttcgccgtcccgcatccaaacgcccccagcagcccttcccctgccccagtgccccgtcgcaactggcagcagcagcgaccagcgactcccccaactcgccggccaccagtagttccctgcttccccatcccatccacacacaccgcacaccaaccaaccccaccacgccaacgtccgggaccaaactctgatccccacc

SEQ ID NO: 86 - DNA, 23 bp, Sorghum bicolor
  misc_feature (21)..(21): n is a, c, g, or t
agccgccgggcgcccgcgtcngg

SEQ ID NO: 87 - DNA, 23 bp, Sorghum bicolor
  misc_feature (21)..(21): n is a, c, g, or t
gagccgccgggcgcccgcgtngg

SEQ ID NO: 88 - DNA, 23 bp, Sorghum bicolor
  misc_feature (21)..(21): n is a, c, g, or t
ggcggctcttcacgcgcgcgngg

SEQ ID NO: 89 - DNA, 23 bp, Sorghum bicolor
  misc_feature (21)..(21): n is a, c, g, or t
ggcctccacgcgcgcgtgaanag

SEQ ID NO: 90 - DNA, 23 bp, Sorghum bicolor
  misc_feature (21)..(21): n is a, c, g, or t
cggcgtcgaggtcgggcacgnag

SEQ ID NO: 91 - DNA, 23 bp, Sorghum bicolor
  misc_feature (21)..(21): n is a, c, g, or t
ttcttccgcgccgtcagggcnag

SEQ ID NO: 92 - DNA, 23 bp, Sorghum bicolor
  misc_feature (21)..(21): n is a, c, g, or t
gtcctcgtcgcgcccatgtgnag

SEQ ID NO: 93 - DNA, 23 bp, Sorghym bicolor
  misc_feature (21)..(21): n is a, c, g, or t
ccaggctcggcaccgtcgtcnag

SEQ ID NO: 94 - DNA, 23 bp, Sorghum bicolor
  misc_feature (21)..(21): n is a, c, g, or t
tacagggcgcggctgacttcngg

SEQ ID NO: 95 - DNA, 23 bp, Sorghum bicolor
  misc_feature (21)..(21): n is a, c, g, or t
cgacaggtatgtcacggtgcnag

SEQ ID NO: 96 - DNA, 1117 bp, Sorghum bicolor
atgcaggcggacggggacgcgccggcgccggcgccggccgtccacttctggggcgagcacccggccacggaggcggagttctacgcggcgcacggcgcggagggcgagccctcctacttcaccacgcccgacgcgggcgcccggcggctcttcacgcgcgcgtggaggccccgcgcgcccgagcggcccagggcgctcgtcttcatggtccacggctacggcaacgacgtcagctggacgttccagtccacggcggtcttcctcgcgcggtccgggttcgcctgcttcgcggccgacctcccgggccacggccgctcccacggcctccgcgccttcgtgcccgacctcgacgccgccgtcgccgacctcctcgccttcttccgcgccgtcagggcgagggaggagcacgcgggcctgccctgcttcctcttcggggagtccatgggcggggccatctgcctgctcatccacctccgcacgcggccggaggagtgggcgggggcggtcctcgtcgcgcccatgtgcaggatctccgaccggatccgcccgccgtggccgctgccggagatcctcaccttcgtcgcgcgcttcgcgcccacggccgctatcgtgcccaccgccgacctcatcgagaagtccgtcaaggtgcccgccaagcgcatcgttgcagcccgcaaccctgtgcgctacaacggtcgccccaggctcggcaccgtcgtcgagctgttgcgtgccaccgacgagctgggcaagcgtctcggcgaggtcagcatcccgttccttgtcgtgcacggcagcgccgacgaggttactgacccggaagtcagccgcgccctgtacgccgccgccgccagcaaggacaagactatcaagatatacgacgggatgctccactccttgctatttggggaaccggacgagaacatcgagcgtgtccgcggcgacatcctggcctggctcaacgagagatgcacaccgccggcaactccctggcaccgtgacatacctgtcgaataagcattccaggctgttcagattccgatgtatcgattacacaagaaaattggtttcatgtacaacgattcttatactatacgctatatacttggtcgtattt

SEQ ID NO: 97 - DNA, 23 bp, Sorghum bicolor
ttcttccgcgccgtcagggcgag

SEQ ID NO: 98 - DNA, 23 bp, Sorghum bicolor
cgacaggtatgtcacggtgccag

SEQ ID NO: 99 - DNA, 406 bp, Sorghum bicolor
tccgcgccttcgtgcccgacctcgacgccgccgtcgccgacctcctcgccttcgaggtcggccgcgaagcaggcgaacccggaccgcgcgaggaagaccgccgtggactggaacgtccagctgacgtcgttgccgtagccgtggaccatgaagacgagcgccctgggccgctcgggcgcgcggggcctccacgcgcgcgtgaagagccgccgggcgcccgcgtcgggcgtggtgaagtaggagggctcgccctccgcgccgtgcgccgcgtagaactccgcctccgtggccgggtgctcgccccagaagtggacggccggcgccggcgccggcgcgtccccgtccgcctgcattcgaataagcattccaggctgttcagattccgatgtatcgattacacaagaaa

(19) United States
(12) Patent Application Publication
(10) Pub. No.: US 2015/0291969 A1
(43) Pub. Date: Oct. 15, 2015

(54) COMPOSITIONS FOR REDUCED LIGNIN CONTENT IN SORGHUM AND IMPROVING CELL WALL DIGESTIBILITY, AND METHODS OF MAKING THE SAME

(71) Applicant: Chromatin, Inc., Chicago, IL (US)

(72) Inventors: Ramesh B. Nair, Naperville, IL (US); Hyeran Lee, Champaign, IL (US)

(21) Appl. No.: 14/611,168

(22) Filed: Jan. 30, 2015

Related U.S. Application Data
(60) Provisional application No. 61/933,582, filed on Jan. 30, 2014.
(60) Provisional application No. 62/107,336, filed on Jan. 23, 2015.

(51) Int. Cl. C12N 15/82

(52) U.S. Cl. CPC C12N 15/8218

(57) ABSTRACT

RNAi vectors comprising a fragment of the SbCSE polynucleotide sequence and transgenic plants, e.g. transgenic sorghum plants, comprising said RNAi vectors are described. Aspects of the technology are further directed to methods of using the RNAi vectors of the present technology to silence SbCSE gene expression or activity in a transgenic plant, such as a transgenic sorghum plant. Silencing the SbCSE gene leads to reduced lignin content in a transgenic plant.

### CROSS-REFERENCE TO RELATED APPLICATION

The present application claims priority to U.S. Provisional Patent Application No. 61/933,582, filed Jan. 30, 2014, entitled “COMPOSITIONS FOR REDUCED LIGNIN CONTENT IN SORGHUM AND IMPROVING CELL WALL DIGESTIBILITY, AND METHODS OF MAKING THE SAME,” and U.S. Provisional Patent Application No. 62/107,336, filed Jan. 23, 2015, entitled GENE MODIFICATION-MEDIATED METHODS FOR GENERATING DOMINANT TRAITS IN EUKARYOTIC SYSTEMS”, which is incorporated herein by reference in its entirety.

### TECHNICAL FIELD

The present technology relates generally to reduced lignin sorghum compositions and methods of making the same in sorghum. By reducing lignin content, forage quality is improved, as well as cellulosic biomass feedstock characteristics.

### SEQUENCE LISTING

The instant application contains a Sequence Listing which has been submitted in ASCII format via EFS-Web and is hereby incorporated by reference in its entirety. Said ASCII copy, created on Jan. 30, 2015, is named 80829-8011US01_ST25.TXT and is 95,200 bytes in size.

### BACKGROUND

Sorghum

Sorghum (such as the commercially common Sorghum bicolor) is a tropical grass that can be grouped into three basic types: (i) grain, (ii) forage, and (iii) sweet sorghum (Monk, 1980). Over 22,000 varieties of sorghum exist throughout the world (Jackson and al, 1980). Sorghum-sudangrass hybrids are intermediate in plant size between sorghum and sudangrass. Sorghum is indigenous to Africa.

Sorghum has many advantageous biological characteristics, including a high photosynthetic rate and high drought tolerance. Sorghum can grow under intense light and heat. In addition, sorghum plants have a waxy surface which reduces internal moisture loss and facilitates drought resistance.

Compared to corn, sorghum suffers harsh environmental conditions successfully, including especially low water and high heat situations (Bennett et al., 1990). However, sorghum grain yields are typically lower than corn, which limits adoption of sorghum cultivation in many corn-growing regions.

Sorghum Forage

Sorghum forage can be used to feed animals, as fuel for biopower plants (“green coal”) and in cellulosic ethanol processes, among other uses.

Sorghum for Feed (Undersander and Lane, 2001)

Sorghums, sorghum-sudangrass hybrids and sudangrasses grown for forage are most appropriately compared with corn silage in feed value. Table 1 lists representative feed values for the various classes of sorghum and sudangrass forages. Corn silage is also included in this table for reference. Table 2 shows the values of Table 1 as a percentage of corn silage.

While generally similar to corn silage for beef cattle and sheep, there are some interesting differences. Sudangrass grazed in its early vegetative stage contains as much available energy as corn silage and considerably more protein. Mature sudangrasses and most sorghum and sudangrass silages are 15-20% lower in available energy than corn silage. Crude protein levels are similar to corn silage, but they are variable and depend in part on available nitrogen.

Calcium and phosphorus levels are higher than corn silage, and the calcium-phosphorus ratio is more optimal. Sorghum and sudangrass contain relatively high levels of potassium. Brown mid-rib (bmr) sorghums are considered to be more digestible.

Sorghum for Cellulosic Ethanol

Lignin inherent in sorghum makes it hard to digest, especially in cellulosic ethanol processes, where the cell wall needs to be broken down to allow full access of the cellulose to the enzymes of the reaction.

Lignin is a phenolic compound and are polymers of p-coumaryl, coniferyl, and sinapyl alcohols and is the second most abundant compound on Earth (Raven et al., 1999). Lignin has several roles: (1) adds to the compressive strength and stiffness plant cell walls; (2) “water proofs” cell walls and consequently aids in the upward transport of water in the xylem; (3) protects plants in case of fungal attack by increasing cell wall resistance to fungal enzymes and diffusion of fungal toxins and enzymes (Raven et al., 1999).

To produce cellulosic ethanol, biomass, such as sorghum biomass, requires that the cell wall portion (the lignocellulose) be pretreated to “loosen” the structure of the cell wall (van der Weijde et al., 2013). This process consists of applying heat, pressure, and chemicals in an attempt to disrupt the cross-links in the cell walls, thus allowing access to the polysaccharides of the cell wall to the enzymes of the cellulosic bioethanol production. The quality of the biomass is important; two of the most important factors are maximizing lignocellulose yield in a sustainable and cost-effective way, and improving the conversion efficiency of lignocellulosic biomass into ethanol (van der Weijde et al., 2013). However, efforts to improve conversion have often ignored biomass composition (van der Weijde et al., 2013). There are, however, studies that have concentrated on lignin's effect in conversion efficiency. For example, when brown midrib mutants in maize and sorghum is assayed for conversion, enzymatic digestibility is improved compared to wild type ((van der Weijde et al., 2013), citing (Dien et al., 2009; Saballos et al., 2008; Sattler et al., 2010; Sattler et al., 2012; Vermerris et al., 2007; Wu et al., 2011)). Similarly, studies in sugarcane, corn and switchgrasss that transgenically down-regulate monolignol biosynthesis genes also improves enzymatic digestibility ((van der Weijde et al., 2013), citing (Fu et al., 2011a; Fu et al., 2011b; Jung et al., 2012; Park et al., 2012; Saathoff et al., 2011). Finally, studies that alter lignin composition (or study natural variants that have altered lignin compared to wild type) can also increase digestibility ((van der Weijde et al., 2013), citing (Fornale et al., 2012; Jung et al., 2012; Saballos et al., 2008; Sattler et al., 2012; Vermerris et al., 2007)).

TABLE 1
Forage Composition of Sorghum Types (expressed as 100% dry matter basis) (Undersander and Lane, 2001)

| | DM¹ | TDN² | NEG³ | NEM⁴ | CP⁵ | EE⁶ | Ca | P | K | NDF⁷ | ADF⁸ |
|---|---|---|---|---|---|---|---|---|---|---|---|
| Grain Sorghum - silage | 30 | 50 | 1.31 | 0.74 | 7.5 | 3.0 | 0.35 | 0.21 | 1.37 | n/a | 38 |
| Forage Sorghum - sorgo | 27 | 58 | 1.24 | 0.68 | 6.2 | 2.5 | 0.34 | 0.17 | 1.12 | n/a | n/a |
| Sudan grass - fresh, early vegetative | 18 | 70 | 1.63 | 1.03 | 16.8 | 3.9 | 0.43 | 0.41 | 2.14 | 55 | 29 |
| Sudan grass - fresh, mid-bloom | 23 | 63 | 1.41 | 0.83 | 8.8 | 1.8 | 0.43 | 0.36 | 2.14 | 65 | 40 |
| Sudan grass-hay, sun-cured | 91 | 56 | 1.18 | 0.61 | 8.0 | 1.8 | 0.55 | 0.30 | 1.87 | 68 | 42 |
| Sudan grass-silage | 28 | 55 | 1.14 | 0.58 | 10.8 | 2.8 | 0.46 | 0.21 | 2.25 | n/a | 42 |
| Corn - silage (well-eared) | 33 | 70 | 1.63 | 1.03 | 8.1 | 3.1 | 0.23 | 0.22 | 0.96 | 51 | 28 |

¹Dry Matter
²Total Digestible Nutrient
³Net Energy for Gain
⁴Net Energy for Maintenance
⁵Crude Protein
⁶Ether Extract (measure of lipid content)
⁷Neutral detergent fiber (measure of digestibility)
⁸Acid detergent fiber (measure of cellulose and lignin)

TABLE 2
Forage Composition of Sorghum Types Expressed as Percentage of Corn Silage (derived from Table 1)

| | DM | TDN | NEG | NEM | CP | EE | Ca | P | K | NDF | ADF |
|---|---|---|---|---|---|---|---|---|---|---|---|
| Grain Sorghum - silage | 90.91 | 71.43 | 80.34 | 71.84 | 92.59 | 96.77 | 152.17 | 95.45 | 142.71 | n/a | 135.71 |
| Forage Sorghum - sorgo | 81.82 | 82.86 | 76.07 | 66.02 | 76.54 | 80.65 | 147.83 | 77.27 | 116.67 | n/a | n/a |
| Sudan grass - fresh, early vegetative | 54.55 | 100 | 100 | 100 | 207.41 | 125.81 | 186.96 | 186.36 | 222.92 | 107.84 | 103.57 |
| Sudan grass - fresh, mid-bloom | 69.70 | 90 | 86.50 | 80.58 | 108.64 | 58.06 | 186.96 | 163.63 | 222.92 | 127.45 | 142.86 |
| Sudan grass-hay, sun-cured | 275.76 | 80 | 72.39 | 59.22 | 98.77 | 58.06 | 239.13 | 136.36 | 194.79 | 133.33 | 150 |
| Sudan grass-silage | 84.85 | 78.57 | 69.94 | 56.31 | 133.33 | 90.32 | 200 | 95.45 | 234.38 | n/a | 150 |

A novel gene (caffeoyl shikimate esterase; CSE) that is involved in lignin biosynthesis has been recently identified in Arabidopsis (Vanholme et al., 2013). An Arabidopsis mutant that is knocked out or knocked down showed reduced level of lignin and improved cell wall digestibility (Vanholme et al., 2013). The general applicability of Vanholme et al.'s findings beyond Arabidopsis is uncertain.

### SUMMARY

Various aspects of the present disclosure provide methods and compositions for altering, modifying or silencing expression of one or more gene products. In one aspect, the present disclosure can be used to modify the expression of the caffeoyl shikimate esterase gene (SbCSE) in Sorghum. For example, in some embodiments, transgenic technology, such as RNAi vectors comprising one or more selected nucleotide sequences, can be used to silence SbCSE gene expression. Other embodiments are directed to methods and compositions for modifying an endogenous gene loci, such as the SbCSE gene in a manner that reduces and/or silences expression of the SbCSE gene. Accordingly, aspects of the present technology can be used for suppressing and/or silencing expression of the SbCSE gene in Sorghum in a manner that reduces lignin biosynthesis, reduces a level of lignin present in the Sorghum plant cell wall and/or improves cell wall digestibility.

One aspect of the present technology provides for an RNAi vector comprising a SbCSE polynucleotide, SbCSE sequence variant polynucleotide, a fragment of at least 20 contiguous nucleotides of a SBCSE polynucleotide or a fragment of at least 20 contiguous nucleotides of a SbCSE sequence variant polynucleotide. These RNAi vectors can facilitate silencing of the SbCSE gene in transgenic plant cells and in transgenic plants which are transformed with the RNAi vectors of the present technology. For example, silencing of the SbCSE gene is accomplished by reducing the level of SbCSE mRNA transcript in the transgenic plant or transgenic plant cell through expression of the RNAi vector in said plant or plant cell.

The RNAi vectors of the present technology comprise a polynucleotide having at least 70%, sequence identity with a nucleic acid sequence selected from the group consisting of SEQ ID NOs:6, 11-13, 49, 51, 53, 55-58, 62 and 63. In addition, the present technology provides for RNAi vectors comprising at least 75%, 80%, 85%, 90%, 95%, 96%, 97%, 98% or 99% sequence identity with a nucleic acid sequence selected from the group consisting of SEQ ID NOs: 6, 11-13, 49, 51, 53, 55-58, 62 and 63. The present technology also provides for RNAi vectors comprising a polynucleotide having a nucleic acid sequence of SEQ ID NO: 6, 11-13, 49, 51, 53, 55-58, 62 and 63 or a fragment thereof which is at least 20 contiguous nucleotides. The RNAi vectors may comprise a polynucleotide having a nucleic acid sequence that is a fragment of at least 25, 30, 35, 40, 45, 50, 75, 100, 110, 120, 140, 150, 160, 170, 180, 190, 200, 204, 220, 250, 300, 350, 359, 360, 367, 400 or 500 contiguous nucleotides of SEQ ID NO: 6, 11-13, 49, 51, 53, 55-58, 62 and 63. The RNAi vectors may comprise a polynucleotide having a nucleic acid sequence that is a fragment of at least 25, 30, 35, 40, 45, 50, 75, 100, 110, 120, 140, 150, 160, 170, 180, 190, 200, 204, 220, 250, 300, 350, 359, 360, 367, 400 or 500 contiguous nucleotides of a SbCSE polynucleotide sequence variant such as a nucleotide sequence that is at least 70%, 90% or 95% identical to SEQ ID NO: 6, 11-13, 49, 51, 53, 55-58, 62 and 63.

The RNAi vectors of the present technology also comprise a polynucleotide having at least 70%, sequence identity with a nucleic acid sequence selected from the group consisting of SEQ ID NOs:14-19, 59, 60, and 61. In addition, the present technology provides for RNAi vectors comprising at least 75%, 80%, 85%, 90%, 95%, 96%, 97%, 98% or 99% sequence identity with a nucleic acid sequence selected from the group consisting of SEQ ID NOs: 14-19, 59, 60, and 61. The present technology also provides for RNAi vectors comprising a polynucleotide having a nucleic acid sequence of SEQ ID NO: 14-19, 59, 60, and 61 or a fragment thereof which is at least 20 contiguous nucleotides. The RNAi vectors may comprise a polynucleotide having a nucleic acid sequence that is a fragment of at least 25, 30, 35, 40, 45, 50, 75, 100, 110, 120, 140, 150, 160, 170, 180, 190, 200, 204, 220, 250, 300, 350, 359, 360, 367, 400 or 500 contiguous nucleotides of SEQ ID NO: 14-19, 59, 60, and 61. The RNAi vectors may comprise a polynucleotide having a nucleic acid sequence that is a fragment of at least 25, 30, 35, 40, 45, 50, 75, 100, 110, 120, 140, 150, 160, 170, 180, 190, 200, 204, 220, 250, 300, 350, 359, 360, 367, 400 or 500 contiguous nucleotides of a SbCSE polynucleotide sequence variant such as a nucleotide sequence that is at least 70%, 90% or 95% identical to SEQ ID NO: 14-19, 59, 60, and 61.

The present technology also provides for plant cells comprising any of the RNAi vectors of the present technology. The present technology also provides for a plant part comprising any of the RNAi vectors of the present technology, such plant parts include seeds and stems.

Other aspects of the present technology provide for transgenic plants comprising any of the RNAi vectors disclosed herein. For example, the present technology provides for Sorghum sp. plants comprising any of the RNAi vectors of the present technology. The present technology also provides for Sorghum bicolor plants comprising any of the RNAi vectors of the present technology. In particular, the present technology provides for transgenic plants, such as Sorghum sp. plants and Sorghum bicolor plants, that have the SbCSE gene silenced such the level of SbCSE expression is decreased compared to the level of SbCSE expression in a control, non-transgenic plant, wherein expression is decreased by reducing the level of mRNA transcript in the plant and the decrease is accomplished by any of the RNAi vectors of the present technology. For example, the present technology provides for transgenic plants and plant cells wherein expression of a SbCSE gene is decreased by at least 90% or 95% when compared to a non-transformed plant cell.

The present technology also provides for seeds and other plant parts of a transgenic plant comprising any of the RNAi vectors of the present technology.

The present technology also provides for methods for silencing SbCSE gene in a transgenic plant such as a transgenic Sorghum plant or a transgenic plant cells, such as a transgenic Sorghum plant cell, comprising decreasing the level of SbCSE expression compared to the level of SbCSE expression its level in a control, non-transgenic plant by reducing the level of an mRNA in the transgenic plant, wherein the mRNA is encoded by a polynucleotide having at least 70% sequence identity to a nucleic acid sequence of SEQ ID NO:6, and by expression of an RNAi construct comprising a fragment of at least 20 contiguous nucleotides of a sequence having at least 90% sequence identity to SEQ ID NO:6.

These methods may be carried out with any of the above-described RNAi vectors of the present technology. For example, the methods may be carried out with an RNAi vector comprising a polynucleotide having at least 90% sequence identity to a polynucleotide selected from the group consisting of SEQ ID NOs:11-13, or an RNAi vector comprising a polynucleotide having at least 95% sequence identity to a polynucleotide selected from the group consisting of SEQ ID NOs: 11-13 or an RNAi vector comprising a polynucleotide having at least 98% sequence identity to a polynucleotide selected from the group consisting of SEQ ID NOs: 11-13. The methods of the present technology also may be carried out with an RNAi vector comprising a polynucleotide selected from the group consisting of SEQ ID NOs: 11-13 or a fragment thereof that is at least 20 contiguous nucleotides of any one of SEQ ID NOs: 11-13.

In addition any of the methods described above may further comprise the step of screening the transgenic plants for a reduction of SbCSE expression by comparing the SBCSE expression in the transgenic plant to a control plant.

The present technology also provides for methods of increasing digestibility of a sorghum plant, comprising transgenically reducing lignin compared to a non-transgenic sorghum plant. For example the increasing digestibility step of this method may be accomplished by expression of any one of the RNAi vectors of the present technology in the sorghum plant.

Additional aspects of the technology are directed to methods and compositions for altering, modifying or silencing expression of the SbCSE gene using a gene-editing/gene-modification-mediated approach. For example, gene editing (i.e., gene-modifying) can be accomplished using a variety of molecular techniques, such as CRISPR-Cas, TALEN (Transcription Activator-Like Effector Nucleases) and Zinc Fingers. In a particular example, the CRISPR-Cas9 technology is a genome editing tool that can target genomes in a gene-specific manor in both mammalian and plant systems [1-4]. In another embodiment, Targeted Induced Local Lesions in Genomes (TILLING) can be used to identify sorghum CSE homologue mutants generated via treatment with a chemical mutagenic agent, such as ethyl methanesulfonate (EMS) [5-6]. Using these gene modification systems, Sorghum sp. with reduced lignin biosynthesis can be generated.

Various aspects of the present technology are directed to a method for altering or modifying expression of a CSE homologue in sorghum. In one embodiment, the method can include introducing into a sorghum cell an engineered, non-naturally occurring vector system comprising one or more vectors, wherein the cell contains and expresses DNA molecules encoding the CSE homologue. The one or more vectors can include: a) a first regulatory element operably linked to one or more Clustered Regularly Interspaced Short Palindromic Repeats (CRISPR)-CRISPR associated (Cas) system guide RNAs that hybridize with CSE homologue target sequences in a genomic loci of the DNA molecules encoding the CSE homologue, b) a second regulatory element operably linked to a Type-II Cas9 protein, wherein components (a) and (b) are located on the same or different vectors of the system. Operatively, the guide RNAs target the genomic loci of the DNA molecules encoding the CSE homologue and the Cas9 protein cleaves the genomic loci of the DNA molecules encoding the CSE homologue. As a result, expression of the CSE homologue is altered. In one embodiment, the guide RNAs include a guide sequence fused to a tracr sequence. The Cas9 protein can be, in certain embodiments, codon optimized for expression in the sorghum cell. In a further embodiment, the expression of sorghum CSE homologue is decreased. Those of ordinary skill in the art, such as those familiar with gene-modification methodology, will understand that cleaving of the genomic loci of the DNA molecule encoding the sorghum CSE homologue encompasses cleaving either one or both strands of the DNA duplex.

### BRIEF DESCRIPTION OF THE DRAWINGS

FIG. 1 shows a diagram schematically illustrating a plasmid construct for use in various disclosed methods and in accordance with an embodiment of the present technology.

FIGS. 2A-5C show nucleotide sequence alignments of Sorghum bicolor (SEQ ID NO:6) with identified orthologs (* indicates identity between the sequences). FIGS. 2A-2C show an alignment between S. bicolor (SEQ ID NO:6) and Zea mays (maize; SEQ ID NO:49). FIGS. 3A-3B show an alignment between S. bicolor (SEQ ID NO:6) and Setaria italica (fox millet; SEQ ID NO:51). FIGS. 4A-4C show an alignment between S. bicolor (SEQ ID NO:6) and Oryza sativa (rice; SEQ ID NO:53). FIGS. 5A-5C show an alignment between S. bicolor (SEQ ID NO:6) and Panicum virgatum (switchgrass; SEQ ID NO:55).

FIG. 6A shows a diagram schematically illustrating a method for CRISPR-Cas-mediated gene replacement in accordance with one embodiment of the present technology.

FIG. 6B shows a diagram schematically illustrating a donor arm for performing the method illustrated in FIG. 6A and in accordance with one embodiment of the present technology.

FIG. 6C shows a diagram schematically illustrating a plasmid map for expression of CRISPR guide RNA for performing the method illustrated in FIG. 6A and in accordance with one embodiment of the present technology.

FIG. 6D shows a diagram schematically illustrating a plasmid map for expression of CRISPR guide RNA and Cas9 for performing the method illustrated in FIG. 6A and in accordance with another embodiment of the present technology.

FIG. 6E shows a diagram schematically illustrating double-stranded RNA formation from the transcription product of the edited gene from FIG. 6A and in accordance with an embodiment of the present technology.

FIG. 7 shows a flow diagram illustrating a method for editing a gene in accordance with an aspect of the present technology.

FIG. 8 shows a diagram schematically illustrating targeting and double-stranded RNA formation of the Sorghum bicolor CAD2 gene in accordance with an embodiment of the present technology.

FIG. 9 shows a diagram schematically illustrating a CRISPR/Cas9 targeted double-stand break on site 1 of SbCAD2 in accordance with one embodiment of the present technology.

FIG. 10 illustrates target sequences and donor sequences for gene replacement in the SbCAD2 gene in accordance with one embodiment of the present technology.

FIG. 11 shows a diagram schematically illustrating a method for CRISPR-Cas-mediated gene replacement in accordance with another embodiment of the present technology.

FIG. 12 shows a diagram schematically illustrating a CRISPR/Cas9 targeted double-stand break on site 1 of SbCSE in accordance with another embodiment of the present technology.

FIG. 13 illustrates target sequences and donor sequences for gene replacement in the SbCSE gene in accordance with another embodiment of the present technology.

### DETAILED DESCRIPTION

The following description provides specific details for a thorough understanding of, and enabling description for, embodiments of the technology. However, one skilled in the art will understand that the technology may be practiced without these details. In other instances, well-known components, derivatives, substitutes and functions have not been shown or described in detail to avoid unnecessarily obscuring the description of the embodiments of the disclosure.

Various aspects of the present technology can be used to modify the genotype and phenotype of any eukaryotic organism (e.g., plant, algal, animal). In a particular example, the present methods disclosed herein can be used to reduce the lignin content in sorghum in a manner that improves cell wall digestibility. Accordingly, aspects of the technology can be used for modifying and selecting plants with reduced, suppressed and/or silenced expression of a CSE homologue, using either a transgenic (e.g., RNAi) approach and/or a gene-modification approach (CRISPR-Cas, TALENs, zinc fingers, etc.). Other embodiments include recovery and identification of ethyl methanesulfonate (EMS)-derived sorghum CSE homologue mutants using TILLING.

### I. INTRODUCTION

As described in more detail in this disclosure, homology searches have revealed the existence of a CSE homologous gene in Sorghum bicolor, named SbCSE. As described herein, and in accordance with aspects of the present technology, mutation of the SbCSE homologue leads to reduction or loss of function resulting in a reduction or recomposing of lignin in sorghum, thereby improving sorghum's digestibility for both livestock and industrial processes.

In embodiments of the present technology, proof of identifying SbCSE is accomplished by RNAi-mediated down-regulation of the candidate gene, which, depending on the degree of penetrance achieved among different transgenic events, should result in a range of morphological phenotypes consistent with disruption of lignin biosynthesis in sorghum. In addition to inducing post-transcriptional gene silencing by RNAi, artificial microRNAs (amiRNAs) can be used to specifically target one or more CSE functional homologues, including SbCSE (Eamens and Waterhouse, 2011; Ossowski et al., 2008; Schwab et al., 2006; Warthmann et al., 2008; Waterhouse and Helliwell, 2003).

Alternatively, targeted mutagenesis can be used to effect a complete loss of function of the candidate gene via deletion, substitution, or insertion of DNA in the gene or its regulatory elements, (Curtain et al., 2012; Gao et al., 2010; Lloyd et al., 2005; Voytas, 2013) which results in quantitative loss of lignin or lignin components. In certain embodiments, gene editing (i.e., gene-modifying) can be accomplished using a variety of molecular techniques, such as CRISPR-Cas9, TALEN (Transcription Activator-Like Effector Nucleases) and Zinc Fingers.

Moreover, in a combination of these approaches, targeted mutagenesis may be used to replace a portion of SbCSE with DNA sequences that cause its transcript to assume a hairpin structure which acts as an RNAi or amiRNA that now causes post-transcriptional silencing of that gene and its homologues, for example in a cross intended to make a hybrid seed. Similarly, an endogenous miRNA locus could be modified by targeted mutagenesis to add, or replace a native sequence with a SbCSE-homologous region resulting in an amiRNA at that locus which acts to post-transcriptionally silence SbCSE and/or its homologues.

RNAi-, miRNA-, or amiRNA-based constructs act as dominant traits, which allows for accelerated trait assessment, for example in a range of test crosses designed to discover modifiers. Moreover, as a dominant-acting trait, both hybrid seed production and inbred development are simplified by use of RNAi or amiRNA. In hybrid seed production, only one inbred parent needs to carry the trait for its expression in F1 seed, which creates flexibility in testing and production of new hybrid combinations. Similarly, development and genetic improvement of inbred parent lines is simplified because only one parental lineage requires conversion and introgression of the trait.

### II. MAKING AND USING ASPECTS OF THE PRESENT TECHNOLOGY

### Note: Definitions are Found at the End of the Detailed Description, Before the Examples; a Table of Selected Abbreviations is Found after the Examples

For reference, the identity of the SEQ ID NOs is shown below:

| SEQ ID NO: | Sequence |
|---|---|
| 1 (aa), 7 (nts) | Arabidopsis thaliana CSE |
| 2 (aa), 6 (nts) | Sorghum bicolor CSE (SbCSE) |
| 3-5 (aa), 8-10 (nts) | S. bicolor CSE homologues of A. thaliana CSE |
| 11 | 5′UTR and 5′-CDS of SbCSE |
| 12 | Central portion of SbCSE |
| 13 | 3′-UTR and 3′-CDS of SbCSE |
| 14-19 | SbCSE RNAi cassettes |
| 20-21 | Vector backbone |
| 22-45 | Event screening primers |
| 46-47 | SbCSE RT-PCR primers |
| 48 (aa), 49 (nts) | Maize CSE (ZmCSE) |
| 50 (aa), 51 (nts) | Setaria italica (fox millet) CSE (SiCSE) |
| 52 (aa), 53 (nts) | Oryza sativa (rice) CSE (OsCSE) |
| 54 (aa), 55 (nts) | Panicum virgatum (switchgrass) CSE (PvCSE) |
| 56 | ZmCSE cds |
| 57 | SiCSE cds |
| 58 | OsCSE cds |
| 59 | ZmCSE RNAi cassette |
| 60 | SiCSE RNAi cassette |
| 61 | OsCSE RNAi cassette |
| 62 | SbCSE promoter |
| 63 | 3′ SbCSE (terminator) |

A. Identification of a CSE Functional Homologue in Sorghum

The polypeptide sequence for CSE from Arabidopsis thaliana (SEQ ID NO:1) is shown in Table 3, while Table 4 shows the polynucleotide sequence encoding SEQ ID NO:1 (SEQ ID NO:7). Using the polypeptide sequence, the sorghum sequence databases were queried using standard procedures and candidate genes were identified. Of these candidate genes, the SbCSE locus was chosen to be the gene Sb02g036570. The SbCSE polynucleotide sequence (SEQ ID NO:6) and the corresponding polypeptide sequence (SEQ ID NO:2) are shown in Tables 5 and 6, respectively.

TABLE 3
Arabidopsis thaliana CSE, polypeptide sequence
(SEQ ID NO: 1)

| | |
|---|---|
| MPSEAESSAN SAPATPPPPP NFWGTMPEEE YYTSQGVRNS KSYFETPNGK LFTQSFLPLD | 60 |
| GEIKGTVYMS HGYGSDTSWM FQKICMSFSS WGYAVFAADL LGHGRSDGIR CYMGDMEKVA | 120 |
| ATSLAFFKHV RCSDPYKDLP AFLFGESMGG LVTLLMYFQS EPETWTGLMF SAPLFVIPED | 180 |
| MKPSKAHLFA YGLLFGLADT WAAMPDNKMV GKAIKDPEKL KIIASNPQRY TGKPRVGTMR | 240 |
| ELLRKTQYVQ ENFGKVTIPV FTAHGTADGV TCPTSSKLLY EKASSADKTL KIYEGMYHSL | 300 |
| IQGEPDENAE IVLKDMREWI DEKVKKYGSK TA | 332 |

TABLE 4
Arabidopsis thaliana CSE, polynucleotide sequence
(SEQ ID NO: 7)

| | |
|---|---|
| atgccgtcgg aagcggagag ctcagcgaat tcagctccgg caactccgcc accaccaccg | 60 |
| aatttctggg gaaccatgcc ggaggaagag tactacactt cacaaggagt acgtaacagc | 120 |
| aaatcatact tcgaaacacc aaacggcaag ctcttcactc agagcttctt accattagat | 180 |
| ggtgaaatca aaggcactgt gtacatgtct catggatacg gatccgatac aagctggatg | 240 |
| tttcagaaga tctgtatgag tttctctagt tggggttacg ctgttttcgc cgccgatctt | 300 |
| ctcggtcacg gccgttccga tggtatccgc tgctacatgg gtgatatgga gaaagttgca | 360 |
| gcaacatcat tggctttctt caagcatgtt cgttgtagtg atccatataa ggatcttccg | 420 |
| gcttttctgt ttggtgaatc gatgggaggt cttgtgacgc ttttgatgta ttttcaatcg | 480 |
| gaacctgaga cttggaccgg tttgatgttt tcggctcctc tctttgttat ccctgaggat | 540 |
| atgaaaccaa gcaaggctca tctttttgct tatggtctcc tctttggttt ggctgatacg | 600 |
| tgggctgcaa tgccggataa taagatggtt gggaaggcta tcaaggaccc tgaaaagctt | 660 |
| aagatcatcg cttctaaccc gcaaagatat acagggaagc ctagagtggg aacaatgaga | 720 |
| gagttactga ggaagactca atacgttcag gagaatttcg ggaaagttac tattccggtg | 780 |
| tttacggcgc acgggacagc ggatggagta acatgtccta catcttcgaa gctactatac | 840 |
| gaaaaagcgt caagcgctga taaaacgttg aagatctatg aagggatgta tcactcgctg | 900 |
| attcaaggag agcctgacga gaacgctgag atagtcttga aggatatgag agagtggatc | 960 |
| gatgagaagg ttaagaagta tggatctaaa accgcttga | 999 |

TABLE 5
Sorghum bicolor CSE (SbCSE), polynucleotide sequence
(SEQ ID NO: 6)

| | |
|---|---|
| atgcaggcgg acggggacgc gccggcgccg gcgccggccg tccacttctg gggcgagcac | 60 |
| ccggccacgg aggcggagtt ctacgcggcg cacggcgcgg agggcgagcc ctcctacttc | 120 |
| accacgcccg acgcgggcgc ccggcggctc ttcacgcgcg cgtggaggcc ccgcgcgccc | 180 |
| gagcggccca gggcgctcgt cttcatggtc cacggctacg gcaacgacgt cagctggacg | 240 |
| ttccagtcca cggcggtctt cctcgcgcgg tccgggttcg cctgcttcgc ggccgacctc | 300 |
| ccgggccacg gccgctccca cggcctccgc gccttcgtgc ccgacctcga cgccgccgtc | 360 |
| gccgacctcc tcgccttctt ccgcgccgtc agggcgaggg aggagcacgc gggcctgccc | 420 |
| tgcttcctct tcggggagtc catgggcggg gccatctgcc tgctcatcca cctccgcacg | 480 |
| cggccggagg agtgggcggg ggcggtcctc gtcgcgccca tgtgcaggat ctccgaccgg | 540 |
| atccgcccgc cgtggccgct gccggagatc ctcaccttcg tcgcgcgctt cgcgcccacg | 600 |
| gccgctatcg tgcccaccgc cgacctcatc gagaagtccg tcaaggtgcc cgccaagcgc | 660 |
| atcgttgcag cccgcaaccc tgtgcgctac aacggtcgcc ccaggctcgg caccgtcgtc | 720 |
| gagctgttgc gtgccaccga cgagctgggc aagcgtctcg gcgaggtcag catcccgttc | 780 |
| cttgtcgtgc acggcagcgc cgacgaggtt actgacccgg aagtcagccg cgccctgtac | 840 |
| gccgccgccg ccagcaagga caagactatc aagatatacg acgggatgct ccactccttg | 900 |
| ctatttgggg aaccggacga gaacatcgag cgtgtccgcg gcgacatcct ggcctggctc | 960 |
| aacgagagat gcacaccgcc ggcaactccc tggcaccgtg acatacctgt cgaataa | 1017 |

TABLE 6
Sorghum bicolor CSE (SbCSE), polypeptide sequence
(SEQ ID NO: 2)

| | |
|---|---|
| MQADGDAPAP APAVHFWGEH PATEAEFYAA HGAEGEPSYF TTPDAGARRL FTRAWRPRAP | 60 |
| ERPRALVFMV HGYGNDVSWT FQSTAVFLAR SGFACFAADL PGHGRSHGLR AFVPDLDAAV | 120 |
| ADLLAFFRAV RAREEHAGLP CFLFGESMGG AICLLIHLRT RPEEWAGAVL VAPMCRISDR | 180 |
| IRPPWPLPEI LTFVARFAPT AAIVPTADLI EKSVKVPAKR IVAARNPVRY NGRPRLGTVV | 240 |
| ELLRATDELG KRLGEVSIPF LVVHGSADEV TDPEVSRALY AAAASKDKTI KIYDGMLHSL | 300 |
| LFGEPDENIE RVRGDILAWL NERCTPPATP WHRDIPVE | 338 |

Similarly, Zea mays (maize), Setaria italica (fox millet), Oryza sativa (rice), and Panicum virgatum (switchgrass) sequence databases were queried using standard procedures and identified orthologous genes. The identified sequences (amino acid and nucleotide, the nucleotide showing the 5′ untranslated regions, the open reading frames, and the 3′ untranslated regions) are shown in Tables 7 and 8 (Z. mays; SEQ ID NOs:48 and 49), 9 and 10 (S. italica; SEQ ID NOs:50 and 51)), 11 and 12 (O. sativa; SEQ ID NOs: 52 and 53)), and 13 and 14 (P. virgatum; SEQ ID NOs:54 and 55).

TABLE 7
Zea mays CSE (ZmCSE), amino acid sequence
(SEQ ID NO: 48)

| | |
|---|---|
| MPADGEALAP AVHFWGEHPA TEAEFYSAHG TEGESSYFTT PDAGARRLFT RAWRPRAPER | 60 |
| PRALVFMVHG YGNDISWTFQ STAVFLARSG FACFAADLPG HGRSHGLRAF VPDLDAAVAD | 120 |
| LLAFFRAVRA REEHAGLPCF LFGESMGGAI CLLIHLRTRP EEWAGAVLVA PMCRISDRIR | 180 |
| PPWPLPEILT FVARFAPTAA IVPTADLIEK SVKVPAKRIV AARNPVRYNG RPRLGTVVEL | 240 |
| LRATDELAKR LGEVSIPFLV VHGSTDEVTD PEVSRALYAA AASKDKTIKI YDGMLHSLLF | 300 |
| GEPDENIERV RGDILAWLNE RCTAQATHRN IPVE | 334 |

TABLE 8
Zea mays CSE (ZmCSE), nucleotide sequence
(SEQ ID NO: 49)

| | |
|---|---|
| ccaccaaggc accaacccga aacgaatcca gtgatttccc ctcccgcatc gaaacgtccc | 60 |
| ccaagcagcc ctgcccggct gcccctgccg cgacgcaact ggcaagcatc cagcatagca | 120 |
| gcgactcccc cgctcgccgg ccagcggcca ccagttccct ttacatccac acacaacgcg | 180 |
| caccacacca caccacccga cgccaacgtc cgggaccaaa ctccgatccc caccactatg | 240 |
| ccggcggacg gggaggcgct ggcgccggcc gttcacttct ggggcgagca cccggccacg | 300 |
| gaggcggagt tctactcggc gcacggcacg gagggcgagt cctcctactt caccacgccc | 360 |
| gacgcgggcg cccggcggct cttcacgcgc gcgtggaggc cccgcgcgcc cgagcggccc | 420 |
| agggcgctcg tgttcatggt ccacggctac ggcaacgaca tcagctggac gttccagtcc | 480 |
| acggcggtct tcctcgcgcg gtccgggttc gcctgcttcg cggccgacct cccgggccac | 540 |
| ggccgctccc acggcctccg cgccttcgtg cccgacctcg acgccgccgt cgctgacctc | 600 |
| ctcgccttct tccgcgccgt cagggcgagg gaggagcacg cgggcctgcc ctgcttcctg | 660 |
| ttcggggagt ccatgggcgg ggccatctgc ctgctcatcc acctccgcac acggccggag | 720 |
| gagtgggcgg gggcggtcct cgtcgctccc atgtgcagga tctccgaccg gatccgcccg | 780 |
| ccgtggccgc tgccggagat tctcaccttc gtcgcgcgct tcgcgcccac ggcggccatc | 840 |
| gtgcccaccg ccgacctcat cgagaagtcc gtcaaggtgc ccgccaagcg catcgttgca | 900 |
| gcgcgcaacc ctgtgcgcta caacggccgt cccaggctcg gcaccgtcgt cgagctgttg | 960 |
| cgtgccaccg acgagctggc caagcgcctc ggcgaagtca gcatcccgtt ccttgtcgtg | 1020 |
| cacggcagca ccgacgaggt taccgacccg gaagtcagcc gcgccctgta cgccgccgcc | 1080 |
| gccagcaagg ataagactat caagatatac gacgggatgc tccactcctt gctatttggg | 1140 |
| gaaccggacg agaacatcga gcgtgtccgt ggggacatcc tggcctggct caatgagaga | 1200 |
| tgcacagccc aggcaactca ccgtaacata cctgtcgaat aagcattcgg atgcatggat | 1260 |
| acacaagaaa aatgtttcat gtacaacgat tgttatatat gctatactca gtatttgact | 1320 |
| gtaaactgtt cggtcaggtt tagtggcttg gatatacaaa atgttggttg cctcatcagt | 1380 |
| gtaaaagaat gctgcaaatg cttgggatcg ataatatcag ctctcttcgg gggctatgga | 1440 |
| tggcaataca aggcgttctc tgccctgtac aagcttggca gaccgaattt tatctcc | 1497 |

TABLE 9
Setaria italica CSE (SiCSE), amino acid sequence
(SEQ ID NO: 50)

| | |
|---|---|
| MPADGDAPAP AVHFWGDHPA TESDYYAAHG AEGEPSYFTT PDEGARRLFT RAWRPRAPAR | 60 |
| PKALVFMVHG YGNDISWTFQ STAVFLARSG FACFAADLPG HGRSHGLRAF VPDLDAAVAD | 120 |
| LLAFFRAVRA REEHAGLPCF LFGESMGGAI CLLIHLRTPP EEWAGAVLVA PMCRISDRIR | 180 |
| PPWPLPEILT FVARFAPTAA IVPTADLIEK SVKVPAKRVI AARNPVRYNG RPRLGTVVEL | 240 |
| LRATDELAKR LGEVTIPFLV VHGSADEVTD PEVSRALYEA AASKDKTIKI YDGMLHSLLF | 300 |
| GELDENIERV RGDILAWLNE KCTLSTSLQR DITVE | 335 |

TABLE 10
Setaria italica CSE (SiCSE), nucleotide sequence
(SEQ ID NO: 51)

| | |
|---|---|
| cgactccccc actcgccggc caccagtagt tccccatcca caccgcatcc ccaccccacg | 60 |
| ccaccgtccg gaaccaaacc ctgatcccca ccatgccggc ggacggggac gcgccggcgc | 120 |
| cggccgtcca cttctggggg gaccacccgg ccacggagtc cgactactac gccgcgcacg | 180 |
| gcgcggaggg cgagccgtcc tacttcacca cgcccgacga gggcgcccgg cggctcttca | 240 |
| cgcgcgcctg gaggccccgc gcgccggcgc gccccaaggc gctcgtcttc atggtccacg | 300 |
| gctacggcaa cgacatcagc tggacgttcc agtccacggc ggtcttcctc gcgaggtccg | 360 |
| ggttcgcctg cttcgcggcc gacctcccgg gccacggccg ctcccatggc ctccgcgcct | 420 |
| tcgtgcccga cctcgacgcc gccgtcgccg acctcctcgc cttcttccgc gccgtcaggg | 480 |
| cgcgggagga gcacgcgggc ctgccctgct tcctcttcgg ggagtccatg ggcggcgcca | 540 |
| tctgcctgct catccacctc cgcacgccgc ccgaggagtg ggcgggggcc gtcctcgtcg | 600 |
| cgcccatgtg caggatctca gaccggatcc gcccgccgtg gccgctgccg gagatcctca | 660 |
| ccttcgtcgc ccggttcgcg cccaccgccg ccatcgtgcc caccgccgac ctcatcgaga | 720 |
| agtccgtcaa ggtgcccgcc aagcgcgtca ttgcggcgcg caaccccgtg cgctacaacg | 780 |
| gccgccccag gctcggcacc gtcgtcgagc tgctgcgcgc caccgacgag ctggccaagc | 840 |
| gcctcggcga ggtcaccatc ccgttcctcg tcgtgcacgg cagcgccgac gaggtcaccg | 900 |
| accccgaagt cagccgcgcc ctgtacgagg ccgcagccag caaggacaag accatcaaga | 960 |
| tatacgacgg gatgctccac tccttgctct tcggggagct ggacgagaac atcgagcgcg | 1020 |
| ttcgtggcga catcctcgcc tggctcaacg agaaatgcac gctgtcaact tccttgcaac | 1080 |
| gtgacataac tgttgaataa | 1100 |

TABLE 11
Oryza sativa CSE (OsCSE), amino acid sequence
(SEQ ID NO: 52)

| | |
|---|---|
| MPDGERHEEA PDVNFWGEQP ATEAEYYAAH GADGESSYFT PPGGRRLFTR AWRPRGDGAP | 60 |
| RALVFMVHGY GNDISWTFQS TAVFLARSGF ACFAADLPGH GRSHGLRAFV PDLDSAIADL | 120 |
| LAFFRSVRRR EEHAGLPCFL FGESMGGAIC LLIHLRTPPE EWAGAVLVAP MCKISDRIRP | 180 |
| PWPLPQILTF VARFAPTLAI VPTADLIEKS VKVPAKRLIA ARNPMRYSGR PRLGTVVELL | 240 |
| RATDELGARL GEVTVPFLVV HGSADEVTDP DISRALYDAA ASKDKTIKIY DGMMHSMLFG | 300 |
| EPDENIERVR ADILAWLNER CTPREEGSFL TIQD | 334 |

TABLE 12
Oryza sativa CSE (OsCSE), nucleotide sequence
(SEQ ID NO: 53)

| | |
|---|---|
| aaaaccgaaa cgccgaacga aacgaatcgt aaactcccct gctgctacgc aacgactccc | 60 |
| caactctccg gccaccacca ccaccacctg ttccccatcc gcacgccacg caccggccca | 120 |
| accgattccc caccatgccg gacggcgagc ggcatgagga ggccccggat gtgaacttct | 180 |
| ggggcgagca gccggcgacg gaggctgagt actacgcggc gcacggcgcg gatggcgagt | 240 |
| cgtcctactt caccccgccg ggcgggcgcc gcctcttcac gcgggcgtgg cggccccgtg | 300 |
| gcgacggcgc gccgcgggcg ctcgtgttca tggtgcacgg ctacggcaac gacatcagct | 360 |
| ggacgttcca gtccacggcc gtcttcctcg cccgctccgg cttcgcctgc ttcgccgccg | 420 |
| acctccccgg ccatggccgc tcccacggcc tccgcgcgtt cgtccccgac ctcgattccg | 480 |
| ccatcgccga cctgctcgcc ttcttccgct ccgtccggcg gcgggaggag cacgccgggc | 540 |
| tgccgtgctt cctgttcggg gagtccatgg gcggggccat ctgcctcctc atccacctcc | 600 |
| gcacgccgcc ggaggagtgg gccggcgccg tgctggtggc gcccatgtgc aagatctccg | 660 |
| accggatccg cccgccatgg ccgctgccgc agatcctcac cttcgtcgcc cgcttcgcgc | 720 |
| ccacgctcgc catcgtcccc accgccgacc tcatcgagaa gtccgtcaag gtgccggcca | 780 |
| agcgcctcat cgccgcgcgc aaccccatgc gctatagcgg ccggccgagg ctcggcaccg | 840 |
| tcgtcgagct gctgcgcgcc accgacgagc tcggcgcccg cctcggcgaa gtcaccgtcc | 900 |
| cgttcctcgt cgtgcacggc agcgccgacg aggtgaccga cccggacatc agccgcgcgc | 960 |
| tgtacgacgc cgccgccagc aaggacaaga ccatcaagat atacgacggg atgatgcact | 1020 |
| ccatgctctt cggggagcct gacgagaaca tcgagcgcgt ccgcgctgac attctcgcgt | 1080 |
| ggctcaacga gagatgcacg ccgagggagg agggcagctt cctgacaata caagattagt | 1140 |
| atccaggatt cactccactc tattcagatt attgtgaagt agcaaatgca caaaaagaat | 1200 |
| gattaaatgt gcaaatttgc agtgattcta tatataaatt tgatgaacat ttgcagtgat | 1260 |
| tctatatata aatttgatga actgctcagt caggtttaca tgatttatgg tataaaatat | 1320 |
| gctaagtctc ctgacc | 1336 |

TABLE 13
Panicum virgatum CSE (PvCSE), amino acid sequence
(SEQ ID NO: 54)

| | |
|---|---|
| MAPPGDPPPA TKYFWGDTPE PDEYYAAQGL RHAESYFQSP HGRLFTHAFH PLAGDVKGVV | 60 |
| FMTHGYGSDS SWLFQTAAIS YARWGYAVFC ADLLGHGRSD GLRGYVGDME AAAAASLAFF | 120 |
| LSVRASAAYA ALPAFLFGES MGGAATLLMY LRSPPSARWT GLVLSAPLLV IPDGMYPSRL | 180 |
| RLFLYGLLFG LADTWAVLPD KRMVGKAIKD PDKLRLIASN PLGYRGAPRV GTMRELVRVT | 240 |
| DLLRESLGEV AAPFLAVHGT DDGVTSPEGS RMLYERASSE DKELILYEGM YHSLIQGEPD | 300 |
| ENRDRVLADM RRWIDERVRR YGPAAAANGG GGKEEPPAP | 339 |

TABLE 14
Panicum virgatum CSE (PvCSE), nucleotide sequence
(SEQ ID NO: 55)

| | |
|---|---|
| agagctcaga ccatcttccc agcacactcc ggcgatggcg ccgcccgggg acccgccgcc | 60 |
| ggcgaccaag tacttctggg gcgacacccc cgagcccgac gagtactacg ccgcgcaggg | 120 |
| gctccggcac gccgagtcct acttccagtc ccctcacggc cgcctcttca cccacgcctt | 180 |
| ccacccgctc gccggcgacg tcaagggcgt cgtcttcatg acccacggct acggttccga | 240 |
| ctcctcgtgg ctcttccaga ccgccgccat cagctacgcg cgctgggggt acgccgtctt | 300 |
| ctgcgccgac ctcctcggcc acggccgctc cgacggcctc cgcgggtacg tcggcgacat | 360 |
| ggaggccgcc gccgcggcgt ccctcgcttt cttcctctcc gtgcgcgcca gcgcggcgta | 420 |
| cgccgcgctc ccggcgttcc tgttcggcga gtccatgggc ggcgccgcca cgctgctcat | 480 |
| gtacctccgc tccccgccgt ccgcgcgctg gacggggctc gtgctctcgg cgccgctcct | 540 |
| cgtcatcccc gacggcatgt acccgtcccg cctccgcctc ttcctgtacg gcctcctctt | 600 |
| cggcctcgcc gacacctggg ccgtgctccc ggacaagagg atggtgggga aggcgatcaa | 660 |
| ggaccccgac aagctgcggc ttatcgcgtc caacccgctc ggctaccgcg gcgcgccgcg | 720 |
| ggtgggcacg atgcgggagc tggtccgcgt gacggatctg ctgcgggaga gcctcgggga | 780 |
| ggtggcggcg ccgttcctcg ccgtgcacgg gacggacgac ggcgtgacct cgccggaggg | 840 |
| gtccaggatg ctgtacgagc gcgcgagcag cgaggacaag gagctcatcc tgtacgaggg | 900 |
| gatgtaccac tcgctcatcc agggggagcc cgacgagaac cgcgaccgcg tgctcgccga | 960 |
| catgcgcagg tggatcgacg agcgcgtgcg ccgctacggc cccgccgccg ccgccaacgg | 1020 |
| gggcggcggc aaggaggagc cgccggcgcc ctgacggtgc ggtgcagtgt tggttgtcac | 1080 |
| ttattcccat cacaactcca ttcctgtttc ttgtttttct tttgggtaat cgctcattcg | 1140 |
| cttgtagttt tacgaagatg atgggcgtcg agtgccatcg actgcaagaa atatctgaac | 1200 |
| tatacctttt gctttcctta aaaaaaaaga gcttttgctt tccttggacc | 1250 |

More details are provided in the Examples below.

B. Silencing SbCSE in Sorghum with RNAi

The present technology includes methods of silencing the SbCSE gene, wherein a sorghum plant is transformed with nucleic acids capable of silencing a SbCSE gene. Silencing SbCSE can be done conveniently by sub-cloning a SbCSE targeting sequence, such as one of the polynucleotides of SEQ ID NOs:11-13 (Table 15), into RNAi vectors or using an RNAi vector comprising a fragment of at least 20 contiguous nucleotides of a sequence having at least 90% sequence identity to SEQ ID NO:6. Exemplary fragments of SEQ ID NO:6 are portions of the 5′UTR and CDS portion of the coding regions such as SEQ ID NO:11, a central portion of the coding region of SEQ ID NO:6 that is not highly conserved such as SEQ ID NO:12, or the 3′CDS and 3′UTR portion of the coding region such as SEQ ID NO:13. Alternatively, the sequences of SEQ ID NOs:56-58 (see Table 24) can be used.

TABLE 15
SbCSE targeting sequences

| SEQ | | |
|---|---|---|
| ID NO: | Sequence | |
| 11 | ccaaccaacc ccaccacgcc aacgtccggg accaaactct gatccccacc atgcaggcgg | 60 |
| | acggggacgc gccggcgccg gcgccggccg tccacttctg gggcgagcac ccggccacgg | 120 |
| | aggcggagtt ctacgcggcg cacggcgcgg agggcgagcc ctcctacttc accacgcccg | 180 |
| | acgcgggcgc ccggcggctc ttcacgcgcg cgtggaggcc ccgcgcgccc gagcggccca | 240 |
| | gg | 242 |
| 12 | gggcgctcgt cttcatggtc cacggctacg gcaacgacgt cagctggacg ttccagtcca | 60 |
| | cggcggtctt cctcgcgcgg tccgggttcg cctgcttcgc ggccgacctc ccgggccacg | 120 |
| | gccgctccca cggcctccgc gccttcgtgc ccgacctcga cgccgccgtc gccgacctcc | 180 |
| | tcgccttctt ccgcgccgtc agggcgaggg aggagcacgc gggcctgccc tgcttcctct | 240 |
| | tcggggagtc | 250 |
| 13 | atcgagcgtg tccgcggcga catcctggcc tggctcaacg agagatgcac accgccggca | 60 |
| | actccctggc accgtgacat acctgtcgaa taagcattcc aggctgttca gattccgatg | 120 |
| | tatcgattac acaagaaaat tggtttcatg tacaacgatt cttatactat acgctatata | 180 |
| | cttggtcgta ttt | 193 |

RNA interference (RNAi) in plants (i.e., post-transcriptional gene silencing (PTGS)) is an example of a broad family of phenomena collectively called RNA silencing (Hannon, 2002). The unifying features of RNA silencing phenomena are the production of small (21-26 nt) RNAs that act as specificity determinants for down-regulating gene expression (Djikeng et al., 2001; Hamilton and Baulcombe, 1999; Hammond et al., 2000; Parrish and Fire, 2001; Parrish et al., 2000; Tijsterman et al., 2002; Zamore et al., 2000) and the requirement for one or more members of the Argonaute family of proteins (or PPD proteins, named for their characteristic PAZ and Piwi domains) (Fagard and Vaucheret, 2000; Hammond et al., 2001; Hutvagner and Zamore, 2002; Kennerdell et al., 2002; Martinez et al., 2002; Pal-Bhadra et al., 2002; Tabara et al., 1999; Williams and Rubin, 2002).

Small RNAs are generated in animals by members of the Dicer family of double-stranded RNA (dsRNA)-specific endonucleases (Bernstein et al., 2001; Grishok et al., 2001; Ketting et al., 2001). Dicer family members are large, multi-domain proteins that contain putative RNA helicase, PAZ, two tandem ribonuclease III (RNase III), and one or two dsRNA-binding domains. The tandem RNase III domains are believed to mediate endonucleolytic cleavage of dsRNA into small interfering RNAs (siRNAs), the mediators of RNAi. In Drosophila and mammals, siRNAs, together with one or more Argonaute proteins, form a protein-RNA complex, the RNA-induced silencing complex (RISC), which mediates the cleavage of target RNAs at sequences with extensive complementarity to the siRNA (Zamore et al., 2000).

In addition to Dicer and Argonaute proteins, RNA-dependent RNA polymerase (RdRP) genes are required for RNA silencing in PTGS initiated by transgenes that overexpress an endogenous mRNA in plants (Zamore et al., 2000), although transgenes designed to generate dsRNA bypass this requirement (Beclin et al., 2002).

Dicer in animals and CARPEL FACTORY (CAF, a Dicer homologue) in plants also generate microRNAs (miRNAs), 20-24-nt, single-stranded non-coding RNAs thought to regulate endogenous mRNA expression (Park et al., 2002). miRNAs are produced by Dicer cleavage of stem-loop precursor RNA transcripts (pre-miRNAs); the miRNA can reside on either the 5′ or 3′ side of the double-stranded stem. Generally, plant miRNAs have far greater complementarity to cellular mRNAs than is the case in animals, and have been proposed to mediate target RNA cleavage via an RNAi-like mechanism (Llave et al., 2002; Rhoades et al., 2002).

In plants, RNAi can be achieved by a transgene that produces hairpin RNA (hpRNA) with a dsRNA region (Waterhouse and Helliwell, 2003). Although antisense-mediated gene silencing is an RNAi-related phenomenon (Di Serio et al., 2001), hpRNA-induced RNAi is more efficient (Chuang and Meyerowitz, 2000). As an example, in an hpRNA-producing vector, the target gene is cloned as an inverted repeat spaced with an unrelated sequence as a spacer and is driven by a strong promoter, such as the ³⁵S CaMV promoter for dicots or the maize ubiquitin 1 promoter for monocots, or alternatively, with a native promoter. When an intron is used as the spacer, essential for stability of the inverted repeat in Escherichia coli, efficiency becomes high: almost 100% of transgenic plants show gene silencing (Smith et al., 2000; Wesley et al., 2001). RNAi can be used against a vast range of targets; 3′ and 5′ untranslated regions (UTRs) as short as 100 nt can be efficient targets of RNAi (Kusaba, 2004).

For genome-wide analysis of gene function, a vector for high-throughput cloning of target genes as inverted repeats, which is based on an LR clonase reaction, is useful (Wesley et al., 2001). Another high-throughput RNAi vector is based on “spreading of RNA targeting” (transitive RNAi) from an inverted repeat of a heterologous 3′ UTR (Brummell et al., 2003a; Brummell et al., 2003b). A chemically regulated RNAi system has also been developed (Guo et al., 2003).

Virus-induced gene silencing (VIGS) is another approach often used to analyze gene function in plants (Waterhouse and Helliwell, 2003). RNA viruses generate dsRNA during their life cycle by the action of virus-encoded RdRP. If the virus genome contains a host plant gene, inoculation of the virus can trigger RNAi against the plant gene. This approach is especially useful for silencing essential genes that would otherwise result in lethal phenotypes when introduced in the germplasm. Amplicon is a technology related to VIGS (Waterhouse and Helliwell, 2003). It uses a set of transgenes comprising virus genes that are necessary for virus replication and a target gene. Like VIGS, amplicon triggers RNAi but it can also overcome the problems of host-specificity of viruses (Kusaba, 2004).

In addition, siRNAs and hpRNAs can be synthesized and then introduced into host cells. The polynucleotides of SEQ ID NOs:11-13 can be prepared by conventional techniques, such as solid-phase synthesis using commercially available equipment, such as that available from Applied Biosystems USA Inc. (Foster City, Calif.; USA), DuPont, (Wilmington, Del.; USA), Genescript USA (Piscataway, N.J., USA), GeneArt/ThermoFisher Scientific (Waltham, Mass., USA) or Milligen (Bedford, Mass.; USA). Modified polynucleotides, such as phosphorothioates and alkylated derivatives, can also be readily prepared by similar methods known in the art. The polynucleotides of SEQ ID NOs:11-13 can also be generated by conventional PCR of genomic DNA from sorghum.

1. RNAi Vectors

Excellent guidance can be found in Preuss and Pikaard regarding RNAi vectors (Preuss and Pikaard, 2004). In some embodiments, RNAi vectors are introduced using Agrobacterium tumefaciens-mediated delivery into plantsd; alternatively, ballistic delivery may be used. Several families of RNAi vectors that use Agrobacterium tumefaciens-mediated delivery into plants are widely available. All share the same overall design, but differ in terms of selectable markers, cloning strategies and other elements (Table 16). A typical design for an RNAi-inducing transgene comprises a strong promoter driving expression of sequences matching the targeted mRNA(s). These targeting sequences are cloned in both orientations flanking an intervening spacer, which can be an intron or a spacer sequence that will not be spliced. For stable transformation, a selectable marker gene, such as herbicide resistance or antibiotic resistance, driven by a plant promoter, is included adjacent to the RNAi-inducing transgene. The selectable marker gene plays no role in RNAi, but allows transformants to be identified by treating seeds, whole plants or cultured cells with herbicide or antibiotic. For transient expression experiments, no selectable marker gene would be necessary. In constructs for use in A. tumefaciens-mediated delivery, the T-DNA is flanked by a left border (LB) and right border (RB) sequence that delimit the segment of DNA to be transferred. For stable transformation mediated by means other than A. tumefaciens, LB and RB sequences are irrelevant (Preuss and Pikaard, 2004).

TABLE 16
Exemplary vectors for stable transformation for hpRNA production

| | pFGC5941 | PMCG161 | pHannibal | pHELLSGATE |
|---|---|---|---|---|
| Organism | Dicots | Monocots | Dicots | Dicots |
| Cloning Method | restriction | restriction | restriction | GATEWAY ® |
| | digest/ligation | digest/ligation | digest/ligation | recombination |
| | | | | (Invitrogen) |
| Bacterial Selection | Kanamycin | chloramphenicol | ampicillin | Spectinomycin and |
| | | | | chloramphenicol |
| Plant Selection | Basta | Basta | (none) | geneticin |
| dsRNA promoter | CaMV 35S | CaMV 35S | CaMV 35S | CaMV 35S |
| Inverted repeat | ChsA intron | Waxy intron | Pdk intron | Pdk intron |
| spacer | | | | |

Two vectors are especially useful, pHANNIBAL and pHELLSGATE (Helliwell et al., 2005; Wesley et al., 2001). pHELLSGATE vectors are also described in U.S. Pat. No. 6,933,146 and US Patent Publication 2005/0164394. The pHANNIBAL vector has an E. coli origin of replication and includes a bacterial selection gene (ampicillin) and a strong promoter (CaMV 35S) upstream of a pair of multiple cloning sites flanking the PDK intron. This structure allows cloning sense and antisense copies of target sequence, separated by the intron. The pHELLSGATE vectors facilitate high-throughput cloning of target sequences directly into an Agrobacterium vector by taking advantage of Gateway® (Life Technologies; Grand Island, N.Y.; USA) recombination technology. The efficiency of pHELLSGATE vectors provides a potential advantage for large scale projects seeking to knock down entire categories of genes. In pHELLSGATE2, the target sequences are incorporated into the T-DNA region (the portion of the plasmid transferred to the plant genome via Agrobacterium-mediated transformation) via the aatB site-specific recombination sequence. pHELLSGATE8 is identical to pHELLSGATE2 but contains the more efficient aatP recombination sites.

Another set of RNAi vectors originally designed for Arabidopsis and maize are freely available through the Arabidopsis Biological Resource Center (ABRC, Ohio State University, Columbus, Ohio) and were donated by the Functional Genomics of Plant Chromatin Consortium (Gendler et al., 2008). Vectors pFGC5941 and pMCG161 include within the T-DNA a selectable marker gene, phosphinothricin acetyl transferase, conferring resistance to the herbicide Basta, and a strong promoter (CaMV 35S) driving expression of the RNAi-inducing dsRNA. Introduction of target sequences into the vector requires two cloning steps, making use of polylinkers flanking a Petunia chalcone synthase intron, an overall design similar to pHANNIBAL. Other ChromDB RNAi vectors, such as pGSA1131, pGSA1165, pGSA1204, pGSA1276, and pGSA1252, pGSA1285, offer kanamycin or hygromycin resistance as plant selectable markers, instead of Basta resistance, and a non-intronic spacer sequence instead of the chalcone synthase intron. The ChromDB vectors are based on pCAMBIA plasmids developed by the Center for Application of Molecular Biology to International Agriculture (CAMBIA; Canberra, Australia). These plasmids have two origins of replication, one for replication in Agrobacterium tumefaciens and another for replication in E. coli. Thus, all cloning steps can be conducted in E. coli prior to transformation (Preuss and Pikaard, 2004).

2. Design of Targeting Sequences (Preuss and Pikaard, 2004)

RNAi vectors are typically designed such that the targeting sequence corresponding to each of the inverted repeats is 300-700 nucleotides in length; however, a stretch of perfect complementarity larger than 14 nucleotides appears absolutely required; 20 nucleotides is a convenient minimum. Success is more easily achieved when the dsRNA targeting sequence is 300-700 nucleotides. Exemplary targeting sequences of the present technology include those of SEQ ID NOs:11-13, 14-19, 49, 515, 53, 55, 56-58, 59-61, and those having at least 90%-99% sequence (e.g., 91%, 92%, 93%, 94%, 95%, 96%, 97%, 98%) identity thereto, as well as any 20 contiguous nucleotides of SEQ ID NO:6 (Table 5) or those having at least 90%-99% sequence (e.g., 91%, 92%, 93%, 94%, 95%, 96%, 97%, 98%) identity thereto.

Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other.

In hpRNAs, one portion of the duplex stem is a nucleic acid sequence that is complementary to the target mRNA. Thus, engineered hpRNA precursors include a duplex stem with two portions and a loop connecting the two stem portions. The two stem portions are about 18 or 19 to about 25, 30, 35, 37, 38, 39, or 40 or more nucleotides in length. In plant cells, the stem can be longer than 30 nucleotides. The stem can include much larger sections complementary to the target mRNA (up to, and including the entire mRNA). The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides.

hpRNAs of the present technology include the sequences of the desired siRNA duplex. The desired siRNA duplex, and thus both of the two stem portions in the engineered RNA precursor, are selected by methods known in the art. These include, but are not limited to, selecting an 18, 19, 20, 21 nucleotide, or longer, sequence from the target gene mRNA sequence from a region 100 to 200 or 300 nucleotides on the 3′ side of the start of translation. In general, the sequence can be selected from any portion of the mRNA from the target gene (such as that of SEQ ID NO:6; Table 5).

3. Inactivation of SbCSE Via Targeted Mutagenesis.

Suitable methods for SbCSE inactivation include any method by which a target sequence-specific DNA-binding molecule can be introduced into a cell. In some embodiments, such agents are, or are operably linked to, a nuclease, which generates double-stranded cuts in the target DNA. Double-stranded DNA breaks initiate endogenous DNA repair mechanisms, primarily non-homologous end-joining, that can result in the deletion or insertion of one, a few, or many nucleotides at the site at which the double-stranded break occurred. These insertions or deletions can result in loss of function of the target gene through introduction of frameshift, nonsense, or missense mutations. In certain embodiments, agents capable of generating double-stranded breaks in target DNA can include meganucleases, homing endonuceases, zinc finger nucleases, or TALENs (Transcription Activator-Like Effector Nucleases) (Curtain et al., 2012; Gao et al., 2010; Lloyd et al., 2005; Voytas, 2013). In other embodiments, methods and compositions for targeted mutagenesis of the SbCSE gene loci, can include CRISPR-Cas gene-editing technologies such as, but not limited to, those described in U.S. Pat. No. 8,697,359, filed Oct. 15, 2013; U.S. patent application Ser. No. 14/211,712, filed Mar. 14, 2014; and International Patent Application No. PCT/US2013/032589, filed Mar. 15, 2013; all of which are incorporated herein by reference in their entireties.

4. Methods for Delivering Polynucleotides to Plants and Plant Cells

Suitable methods include any method by which DNA can be introduced into a cell, such as by Agrobacterium or viral infection, direct delivery of DNA such as, for example, by PEG-mediated transformation of protoplasts (Omirulleh et al., 1993), by desiccation/inhibition-mediated DNA uptake, by electroporation, by agitation with silicon carbide fibers, by acceleration of DNA coated particles, etc. In certain embodiments, acceleration methods include, for example, microprojectile bombardment.

Technology for introduction of DNA into cells is well-known to those of skill in the art. Four general methods for delivering a gene into cells have been described: (1) chemical methods (Graham and van der Eb, 1973; Zatloukal et al., 1992); (2) physical methods such as microinjection (Capecchi, 1980), electroporation (Fromm et al., 1985; Wong and Neumann, 1982) and the gene gun (Fynan et al., 1993; Johnston and Tang, 1994); (3) viral vectors (Clapp, 1993; Eglitis and Anderson, 1988; Eglitis et al., 1988; Lu et al., 1993); and (4) receptor-mediated mechanisms (Curiel et al., 1991; Curiel et al., 1992; Wagner et al., 1992).

Electroporation can be extremely efficient and can be used both for transient expression of cloned genes and for establishment of cell lines that carry integrated copies of the gene of interest. The introduction of DNA by electroporation is well-known to those of skill in the art. In this method, certain cell wall-degrading enzymes, such as pectin-degrading enzymes, are employed to render the target recipient cells more susceptible to transformation by electroporation than untreated cells. Alternatively, recipient cells are made susceptible to transformation by mechanical wounding. To effect transformation by electroporation one can use either friable tissues such as a suspension culture of cells or embryogenic callus, or alternatively one can transform immature embryos or other organized tissues directly. Cell walls are partially degraded of the chosen cells by exposing them to pectin-degrading enzymes (pectolyases) or mechanically wounded in a controlled manner.

Microprojectile bombardment shoots particles coated with the DNA of interest into to plant cells. In this process, the desired nucleic acid is deposited on or in small dense particles, e.g., tungsten, platinum, or 1 micron gold particles, that are then delivered at a high velocity into the plant tissue or plant cells using a specialized biolistics device, such as are available from Bio-Rad® Laboratories (Hercules, Calif.; USA). The advantage of this method is that no specialized sequences need to be present on the nucleic acid molecule to be delivered into plant cells.

For bombardment, cells in suspension are concentrated on filters or solid culture medium. Alternatively, immature embryos, seedling explants, or any plant tissue or target cells can be arranged on solid culture medium. The cells to be bombarded are positioned at an appropriate distance below the microprojectile stopping plate.

Various biolistics protocols have been described that differ in the type of particle or the manner in that DNA is coated onto the particle. Any technique for coating microprojectiles that allows for delivery of transforming DNA to the target cells can be used. For example, particles can be prepared by functionalizing the surface of a gold oxide particle by providing free amine groups. DNA, having a strong negative charge, binds to the functionalized particles.

Parameters such as the concentration of DNA used to coat microprojectiles can influence the recovery of transformants containing a single copy of the transgene. For example, a lower concentration of DNA may not necessarily change the efficiency of the transformation but can instead increase the proportion of single copy insertion events. Ranges of approximately 1 ng to approximately 10 pg, approximately 5 ng to 8 μg or approximately 20 ng, 50 ng, 100 ng, 200 ng, 500 ng, 1 pg, 2 μg, 5 μg, or 7 μg of transforming DNA can be used per each 1.0-2.0 mg of starting 1.0 micron gold particles.

Other physical and biological parameters can be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. Physical parameters such as DNA concentration, microprojectile particle size, gap distance, flight distance, tissue distance, and helium pressure, can be optimized.

The particles delivered via biolistics can be “dry” or “wet.” In the “dry” method, the DNA-coated particles such as gold are applied onto a macrocarrier (such as a metal plate, or a carrier sheet made of a fragile material, such as MYLAR® (biaxially-oriented polyethylene terephthalate) and dried. The gas discharge then accelerates the macrocarrier into a stopping screen that halts the macrocarrier but allows the particles to pass through. The particles are accelerated at, and enter, the plant tissue arrayed below on growth media. The media supports plant tissue growth and development and are suitable for plant transformation and regeneration. These tissue culture media can either be purchased as a commercial preparation, or custom prepared and modified. Examples of such media include Murashige and Skoog (MS), N6, Linsmaier and Skoog, Uchimiya and Murashige, Gamborg's B5 media, D medium, McCown's Woody plant media, Nitsch and Nitsch, and Schenk and Hildebrandt. Those of skill in the art are aware that media and media supplements such as nutrients and growth regulators for use in transformation and regeneration and other culture conditions such as light intensity during incubation, pH, and incubation temperatures can be optimized.

Those of skill in the art can use, devise, and modify selective regimes, media, and growth conditions depending on the plant system and the selective agent. Typical selective agents include antibiotics, such as geneticin (G418), kanamycin, paromomycin; or other chemicals, such as glyphosate or other herbicides.

Agrobacterium-mediated transfer is a widely applicable system for introducing genes into plant cells because the DNA can be introduced into whole plant tissues, thereby bypassing the need for regeneration of an intact plant from a protoplast. Daihy-Yelin et al. provide an overview of Agrobacterium transformation (Dafny-Yelin and Tzfira, 2007). Agrobacterium plant integrating vectors to introduce DNA into plant cells is well known in the art, such as those described above, as well as others (Rogers et al., 1987). Further, the integration of the T-DNA is a relatively precise process resulting in few rearrangements. The region of DNA to be transferred is defined by the border sequences (Jorgensen et al., 1987; Spielmann and Simpson, 1986).

A transgenic plant formed using Agrobacterium transformation methods typically contains a single gene on one chromosome. Homozygous transgenic plants can be obtained by sexually mating (selfing) an independent segregant transgenic plant that contains a single added gene, germinating some of the seed produced and analyzing the resulting plants for the targeted trait or insertion.

In some methods, Agrobacterium carrying the gene of interested can be applied to the target plants when the plants are in bloom. The bacteria can be applied via vacuum infiltration protocols in appropriate media, or even simply sprayed onto the blooms.

For RNA-mediated inhibition in a cell line or whole organism, gene expression can be conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucuronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, basta, and tetracyclin. Depending on the assay, quantitation of the amount of gene expression allows one to determine a degree of inhibition which is greater than 10%, 33%, 50%, 90%, 95%, 99%, or 100% as compared to a cell not treated. Lower doses of injected material and longer times after administration of RNAi agent can result in inhibition in a smaller fraction of cells (e.g., at least 10%, 20%, 50%, 75%, 80%, 85%, 90%, or 95% of targeted cells). Quantitation of gene expression in a cell can show similar amounts of inhibition at the level of accumulation of target mRNA or translation of target protein. As an example, the efficiency of inhibition can be determined by assessing the amount of gene product in the cell; mRNA can be detected with a hybridization probe having a nucleotide sequence outside the region used for the inhibitory double-stranded RNA, or translated polypeptide can be detected with an antibody raised against the polypeptide sequence of that region. Quantitative PCR techniques can also be used.

### DEFINITIONS

“Consisting essentially of a polynucleotide having a % sequence identity” means that the polynucleotide does not substantially differ in length, but in sequence. Thus, a polynucleotide “A” consisting essentially of a polynucleotide having 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to either favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decreases DNA duplex stability. A common approach is to vary the temperature: higher relative temperatures result in more stringent reaction conditions. Ausubel et al. (1987) provide an excellent explanation of stringency of hybridization reactions (Ausubel, 1987).

An “isolated” molecule (e.g., “isolated siRNA” or “isolated siRNA precursor”) refers to a molecule that is substantially free of other cellular material, or culture medium when produced by recombinant techniques, or substantially free of chemical precursors or other chemicals when chemically synthesized.

“Linker” refers to a DNA molecule, generally up to 50 or 60 nucleotides long and composed of two or more complementary oligonucleotides that have been synthesized chemically, or excised or amplified from existing plasmids or vectors. In one embodiment, this fragment contains one, or more than one, restriction enzyme site for a blunt cutting enzyme and/or a staggered cutting enzyme, such as BamHI. One end of the linker is designed to be ligatable to one end of a linear DNA molecule and the other end is designed to be ligatable to the other end of the linear molecule, or both ends may be designed to be ligatable to both ends of the linear DNA molecule

“Non-protein expressing sequence” or “non-protein coding sequence” means a nucleic acid sequence that is not eventually translated into protein. The nucleic acid may or may not be transcribed into RNA. Exemplary sequences include ribozymes or antisense RNA.

“Nucleotide analog” or “altered nucleotide” or “modified nucleotide” refers to a non-standard nucleotide, including non-naturally occurring ribonucleotides or deoxyribonucleotides. In one embodiment, nucleotide analogs are modified at any position so as to alter certain chemical properties of the nucleotide yet retain the ability of the nucleotide analog to perform its intended function. Examples of positions of the nucleotide which can be derivitized include the 5 position, e.g., 5-(2-amino)propyl uridine, 5-bromo uridine, 5-propyne uridine, 5-propenyl uridine, etc.; the 6 position, e.g, 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs (Herdewijn, 2000).

“Operably linked” means a configuration in which a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

“Percent (%) nucleic acid sequence identity” with respect to SbCSE sequence-nucleic acid sequences is defined as the percentage of nucleotides in a candidate sequence that are identical with the nucleotides in the SbCSE sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalig (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Phenotype” or “phenotypic trait(s)” refers to an observable property or set of properties resulting from the expression of a gene. The set of properties may be observed visually or after biological or biochemical testing, and may be constantly present or may only manifest upon challenge with the appropriate stimulus or activation with the appropriate signal.

The term “plant part” includes a pod, root, sett root, shoot root, root primordial, shoot, primary shoot, secondary shoot, tassle, panicle, arrow, midrib, blade, ligule, auricle, dewlap, blade joint, sheath, node, internode, bud furrow, leaf scar, cutting, tuber, stem, stalk, fruit, berry, nut, flower, leaf, bark, wood, epidermis, vascular tissue, organ, protoplast, crown, callus culture, petiole, petal, sepal, stamen, stigma, style, bud, meristem, cambium, cortex, pith, sheath, silk, ovule or embryo. Other exemplary plant parts are a meiocyte or gamete or ovule or pollen or endosperm of any of the preceding plants. Other exemplary plant parts are a seed, seed-piece, embryo, protoplast, cell culture, any group of plant cells organized into a structural and functional unit or propagule.

A “polynucleotide” is a nucleic acid polymer of ribonucleic acid (RNA), deoxyribonucleic acid (DNA), modified RNA or DNA, or RNA or DNA mimetics (such as, PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present technology, are referred to as “analogues.” Oligonucleotides are generally short polynucleotides from about 10 to up to about 160 or 200 nucleotides.

“Polypeptide” is a chain of amino acids connected by peptide linkages. The term “polypeptide” does not refer to a specific length of the encoded product and, therefore, encompasses peptides, oligopeptides, and proteins. The term “exogenous polypeptide” is defined as a polypeptide which is not native to the plant cell, a native polypeptide in which modifications have been made to alter the native sequence, or a native polypeptide whose expression is quantitatively altered as a result of a manipulation of the plant cell by recombinant DNA techniques.

A “promoter” is a DNA sequence that allows the binding of RNA polymerase (including RNA polymerase I, RNA polymerase II and RNA polymerase III from eukaryotes) and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” is a promoter that is operably linked to a gene that is different from the gene to which the promoter is normally operably linked in its native state. Similarly, an “exogenous nucleic acid operably linked to a heterologous regulatory sequence” is a nucleic acid that is operably linked to a regulatory control sequence to which it is not normally linked in its native state.

“Regulatory sequence” refers to any DNA sequence that influences the efficiency of transcription or translation of any gene. The term includes sequences comprising promoters, enhancers and terminators. Similarly, an “exogenous regulatory sequence” is a nucleic acid that is associated with a gene to which it is not normally associated with its native state.

“RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. Oligonucleotides can be linked with linkages which result in a lower rate of hydrolysis of the RNA analog as compared to an RNA molecule with phosphodiester linkages. For example, the nucleotides of the analog can comprise methylenediol, ethylene diol, oxymethylthio, oxyethylthio, oxycarbonyloxy, phosphorodiamidate, phophoroamidate, and/or phosphorothioate linkages. RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

“RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

“RNAi vectors” refer to a construct designed to carry and express an RNA interference polynucleotide in a host cell, such as a sorghum cell, and which will decrease expression of the gene of interest or silence the gene of interest. RNAi vectors include vectors comprising RNAi, microRNAs (miRNAa), hairpin RNA (hpRNA) or artificial microRNA (amiRNA).

“CSE sequence variant polynucleotide” or “CSE sequence variant nucleic acid sequence” means a CSE sequence variant polynucleotide having at least about 60% nucleic acid sequence identity, at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity or at least about 99% nucleic acid sequence identity with the nucleic acid sequence of SEQ ID NOs:6, 49, 51, 53, and 55. Variants do not encompass the native nucleotide sequence.

Ordinarily, CSE sequence variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

A “screenable marker” is a gene whose presence results in an identifiable phenotype. This phenotype may be observable under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. For example, screenable markers of the present technology can include genes that encode fluorescent proteins that are detectable by a visual microscope such as the fluorescent reporter genes DsRed, ZsGreen, ZsYellow, AmCyan, Green Fluorescent Protein (GFP) and modifications of these reporter genes to excite or emit at altered wavelengths. An additional screenable marker gene is lac.

Alternative methods of screening for modified plant cells may involve use of relatively low, sub-killing concentrations of a selection agent (e.g. sub-killing antibiotic concentrations), and also involve use of a screenable marker (e.g., a visible marker gene) to identify clusters of modified cells carrying the screenable marker, after which these screenable cells are manipulated to homogeneity. As used herein, a “selectable marker” is a gene whose presence results in a clear phenotype, and most often a growth advantage for cells that contain the marker. This growth advantage may be present under standard conditions, altered conditions such as elevated temperature, specialized media compositions, or in the presence of certain chemicals such as herbicides or antibiotics. Use of selectable markers is described, for example, in (Broach et al., 1979). Examples of selectable markers include the thymidine kinase gene, the cellular adenine phosphoribosyltransferase gene and the dihydrylfolate reductase gene, hygromycin phosphotransferase genes, the bar gene, neomycin phosphotransferase genes and phosphomannose isomerase, among others. Other selectable markers in the present technology include genes whose expression confer antibiotic or herbicide resistance to the host cell, or proteins allowing utilization of a carbon source not normally utilized by plant cells. Expression of one of these markers should be sufficient to enable the survival of those cells that comprise a vector within the host cell, and facilitate the manipulation of the plasmid into new host cells. Of particular interest in the present technology are proteins conferring cellular resistance to kanamycin, G418, paramomycin, hygromycin, bialaphos, and glyphosate for example, or proteins allowing utilization of a carbon source, such as mannose, not normally utilized by plant cells.

“Small interfering RNA” (“siRNA”) (or “short interfering RNA”) refers to an RNA (or RNA analog) comprising between about 10-50 nucleotides (or nucleotide analogs) that is capable of directing or mediating RNA interference. An effective siRNA can comprise between about 15-30 nucleotides or nucleotide analogs, between about 16-25 nucleotides, between about 18-23 nucleotides, and even about 19-22 nucleotides.

“Sorghum” means Sorghum bicolor (primary cultivated species), Sorghum almum, Sorghum amplum, Sorghum angustum, Sorghum rundinaceum, Sorghum brachypodum, Sorghum bulbosum, Sorghum burmahicum, Sorghum controversum, Sorghum drummondii, Sorghum carinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, and Sorghum vulgare (including but not limited to the variety Sorghum vulgare var. sudanens also known as sudangrass). Hybrids of these species are also of interest in the present technology as are hybrids with other members of the Family Poaceae.

“Specifically hybridize” refers to the ability of a nucleic acid to bind detectably and specifically to a second nucleic acid. Polynucleotides specifically hybridize with target nucleic acid strands under hybridization and wash conditions that minimize appreciable amounts of detectable binding by non-specific nucleic acids.

To hybridize under “stringent conditions” describes hybridization protocols in which nucleotide sequences at least 60% homologous to each other remain hybridized.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

A “targeting” sequence means a nucleic acid sequence of SbCSE sequence or complements thereof can silence a SbCSE gene. Exemplary targeting sequences include SEQ ID NOs:11-13. A target sequence can be selected that is more or less specific for a particular Sorghum

“Transformed,” “transgenic,” “modified,” and “recombinant” refer to a host organism such as a plant into which an exogenous or heterologous nucleic acid molecule has been introduced, and includes meiocytes, seeds, zygotes, embryos, endosperm, or progeny of such plant that retain the exogenous or heterologous nucleic acid molecule but which have not themselves been subjected to the transformation process.

“Transgene” refers to any nucleic acid molecule that is inserted by artifice into a cell, and becomes part of the genome of the organism that develops from the cell. Such a transgene can include a gene that is partly or entirely heterologous (i.e., foreign) to the transgenic organism, or can represent a gene homologous to an endogenous gene of the organism. Transgene also means a nucleic acid molecule that includes one or more selected nucleic acid sequences, e.g., DNAs, that encode one or more engineered RNA precursors, to be expressed in a transgenic organism, e.g., plant, that is partly or entirely heterologous, i.e., foreign, to the transgenic plant, or homologous to an endogenous gene of the transgenic plant, but which is designed to be inserted into the plant's genome at a location that differs from that of the natural gene. A transgene includes one or more promoters and any other DNA, such as introns, necessary for expression of the selected nucleic acid sequence, operably linked to the selected sequence, and can include an enhancer sequence.

Comparing a value, level, feature, characteristic, property, etc. to a suitable control means comparing that value, level, feature, characteristic, or property to any control or standard familiar to one of ordinary skill in the art useful for comparison purposes. A suitable control can be a value, level, feature, characteristic, property, etc. determined prior to performing an RNAi methodology. For example, a transcription rate, mRNA level, translation rate, protein level, biological activity, cellular characteristic or property, genotype, phenotype, etc. can be determined prior to introducing an RNAi agent of the present technology into a cell or organism. A suitable control can be a value, level, feature, characteristic, property, etc. determined in a cell or organism, e.g., a control or normal cell or organism, exhibiting, for example, normal traits. A control can also be a predefined value, level, feature, characteristic, property, etc.

### EXAMPLES

The following examples are meant to only exemplify the present technology, not to limit it in any way. One of skill in the art can envision many variations and methods to practice the present technology.

### Example 1

### Identification of the Sorghum CSE Homologue

The amino acid sequence of Arabidopsis CSE gene (At1g52760; SEQ ID NO:1) was used for identifying sorghum homologues from the Phytozome database (Goodstein et al., 2012). When SEQ ID NO:1 was used to query the sorghum database (Altschul et al., 1997), 15 candidate homologous sorghum proteins that varied in amino acid sequence identity from 43.2-32.0% and protein similarity from 61.0-49.6% over a region of 160-308 amino acids were identified. Among the identified sorghum polypeptide sequences, the amino acid sequence of SEQ ID NO:2 (Table 6) showed the highest protein similarity of 61% and amino acid identity of 42.2%. This polypeptide also had the closest number of amino acids (338) as compared to Arabidopsis CSE protein sequence (332 amino acids). Three other top sorghum homologues (SEQ ID NOs:3-5) had lower amino acid sequence identity of 37.6-35.4% and lower amino acid sequence similarity (55.9-53.6%) with protein sequences of 348-353 amino acids. Thus it is highly likely the sorghum homologue of Arabidopsis CSE is encoded by SEQ ID NO:6 (Table 5). A sequence alignment of SEQ ID NO:2 with three other putative sorghum homologues (SEQ ID NOs:3-5, sequences shown in Table 17) showed that the SEQ ID NO:2 shared only 44.6-43.6% sequence identity at the amino acid level. Thus it is highly likely there is only one homologue of CSE in sorghum, SEQ ID NO:2, encoded by SEQ ID NO:6.

TABLE 17
Putative CSE sorghum homologs

| SEQ | | |
|---|---|---|
| ID NO: | Sequence | |
| 3 | MMDVVYHEEY VRNPRGVQLF TCGWLPPASS SPPKALVFLC HGYGMECSDF MRACGIKLAT | 60 |
| | AGYGVFGIDY EGHGKSMGAR CYIQKFENLV ADCDRFFKSI CDMEEYRNKS RFLYGESMGG | 120 |
| | AVALLLHRKD PTFWDGAVLV APMCKISEKV KPHPVVVTLL TQVEEIIPKW KIVPTKDVID | 180 |
| | SAFKDPVKRE KIRKNKLIYQ DKPRLKTALE LLRTSMDVED SLSEVTMPFF ILHGEADTVT | 240 |
| | DPEVSRALYE RAASTDKTIK LYPGMWHGLT AGEPDENVEL VFSDIVSWLD KRSRHWEQDE | 300 |
| | RARTPPEPEN KHRQAATTKI TRVTSSSGGT ESQRRGSCLC GLGGRPHQQQ CRM | 353 |
| 4 | MEVEYHEEYV RNSRGVQLFT CGWLPVATSP KALVFLCHGY GMECSGFMRE CGMRLAAAGY | 60 |
| | GVFGMDYEGH GKSMGARCYI RSFRRLVDDC SHFFKSICEL EEYRGKSRFL YGESMGGAVA | 120 |
| | LLLHRKDPAF WDGAVLVAPM CKISEKVKPH PVVITLLTQV EDVIPKWKIV PTKQDVIDAA | 180 |
| | FKDPVKREKI RRNKLIYQDK PRLKTALEML RTSMYIEDSL SQVKLPFFVL HGEADTVTDP | 240 |
| | EVSRALYERA ASADKTIKLY PGMWHGLTAG ETDENVEAVF SDIVSWLNQR CRSWTMEDRF | 300 |
| | RKLVPAPAKF IHGDDAVDGK AQTQGRPRRR RPGLLCGLAG RTHHHAEM | 348 |
| 5 | MGRSSSSSGG GGADDGGEVL LDHEYKEEYV RNSRGMNLFA CTWLPAGKRK TPKALVFLCH | 60 |
| | GYAVECGVTM RGTGERLARA GYAVYGLDYE GHGRSDGLQG YVPDFELLVQ DCDEYFTSVV | 120 |
| | RSQSIEDKGC KLRRFLLGES MGGAVALLLD LRRPEFWTGA VLVAPMCKIA DDMRPHPLVV | 180 |
| | NILRAMTSIV PTWKIVPSND VIDAAYKTQE KRDEIRGNPY CYKDKPRLKT AYELLKVSLD | 240 |
| | LEQNLLHQVS LPFLIVHGGA DKVTDPSVSE LLYRSAASQD KTLKLYPGMW HALTSGESPD | 300 |
| | NIHTVFQDII AWLDHRSSDD TDQQELLSEV EQKARHDEQH HQQQDGGNK | 349 |

### Example 2

### Functional Characterization of Sorghum CSE

To confirm the selection of SEQ ID NO:6 as the sorghum CSE homologue, in vitro enzymatic activity is assayed. The open reading frame of top four candidate sorghum CSE genes identified in Example 1; SEQ ID NOs:6, 8-10 are synthesized and cloned into protein expression vector containing histidine (His) tags. The polypeptides are expressed in E. coli or in yeast, and the His-tagged recombinant polypeptides are purified and analyzed for the conversion of caffeoyl shikimate to caffeic acid in vitro. Candidate genes that show caffeoyl shikimate esterase activity are used for down regulation of lignin biosynthesis in sorghum.

### Example 3

### Analysis of Expression Profiles of SbCSE

To understand the expression pattern and localization of SbCSE, a gene expression microarray analysis was performed, examining expression in whole plants as well as specific tissues. We conducted a microarray analysis of putative SbCSE (SEQ ID NO:6) using a microarray dataset from different sorghum tissues that we had previously produced and compared SbCSE's expression to the gene expression pattern of the house-keeping gene SbActin. The results of the microarray analysis of gene expression (shown in Table 18) suggests that the SbCSE is constitutively expressed in various tissues, including both tissues that are rich in primary (seedling shoot, root and stem pith) and secondary cell walls (whole stem and in isolated rind tissues). Thus the constitutive expression of SbSCE in all tissues suggest the role of SbSCE in both primary cell wall and secondary cell wall biosynthesis in sorghum.

TABLE 18
Microarray analysis results (all values are in log2 scale)

| Genotype | | | PI455230 | | R159 | | Atlas | |
|---|---|---|---|---|---|---|---|---|
| Sampled tisssues | | | sbCSE | sbACTIN | sbCSE | sbACTIN | sbCSE | sbACTIN |
| seedling | shoot | all | 8.07 | 12.07 | 8.09 | 11.95 | 8.12 | 11.88 |
| seedling | shoot | all | 8.01 | 11.92 | 8.20 | 11.80 | 8.16 | 12.07 |
| seedling | root | all | 8.10 | 12.93 | 8.26 | 12.87 | 7.79 | 12.66 |
| seedling | root | all | | | | | | |
| leaf | leaf | all | 8.81 | 10.32 | 8.55 | 10.22 | 8.56 | 10.21 |
| shoot | shoot_tip | all | 7.82 | 12.80 | 7.87 | 12.80 | 7.78 | 12.65 |
| stem | internode | top | 8.10 | 12.97 | 7.94 | 12.00 | 8.16 | 12.20 |
| stem | internode | middle | 8.01 | 11.85 | | | 7.77 | 11.82 |
| stem | internode | bottom | 8.11 | 11.87 | 8.00 | 11.35 | 7.85 | 12.01 |
| stem | rind | top | 8.06 | 12.23 | 7.85 | 11.22 | 8.00 | 11.75 |
| stem | rind | middle | 7.87 | 12.26 | | | 7.48 | 11.73 |
| stem | rind | bottom | 7.87 | 12.35 | 7.75 | 10.99 | 7.84 | 12.53 |
| stem | pith | top | 8.61 | 12.07 | 8.07 | 11.13 | 7.51 | 10.28 |
| stem | pith | middle | 8.13 | 10.09 | | | 7.51 | 10.74 |
| stem | pith | bottom | 7.78 | 10.57 | 8.02 | 11.17 | 7.84 | 12.21 |
| stem | rind | all | 8.11 | 10.86 | 8.20 | 11.35 | 8.01 | 11.51 |
| stem | rind | all | 8.18 | 11.87 | | | 8.02 | 11.33 |
| stem | rind | all | 7.96 | 10.56 | | | 8.01 | 11.68 |
| stem | pith | all | 7.98 | 11.82 | 7.99 | 11.27 | 7.72 | 12.33 |
| stem | pith | all | 7.97 | 12.21 | | | 7.93 | 11.77 |
| stem | pith | all | 7.85 | 12.30 | | | 7.65 | 11.47 |
| Genotype | | | PI152611 | | AR2400 | | Fremont | |
| Sampled tisssues | | | sbCSE | sbACTIN | sbCSE | sbACTIN | sbCSE | sbACTIN |
| seedling | shoot | all | 8.25 | 11.73 | 8.16 | 11.93 | 8.07 | 11.93 |
| seedling | shoot | all | 8.26 | 11.83 | 8.23 | 11.98 | 7.95 | 11.87 |
| seedling | root | all | 7.69 | 12.70 | 7.84 | 12.64 | 7.95 | 12.69 |
| seedling | root | all | | | 8.01 | 12.68 | 7.80 | 12.57 |
| leaf | leaf | all | 9.21 | 10.53 | 8.65 | 10.28 | 8.40 | 10.04 |
| shoot | shoot_tip | all | 7.64 | 12.61 | 7.72 | 12.61 | 7.88 | 12.55 |
| stem | internode | top | 7.90 | 13.01 | 8.22 | 13.09 | 7.95 | 10.49 |
| stem | internode | middle | 8.13 | 11.21 | 7.81 | 11.11 | 7.77 | 10.28 |
| stem | internode | bottom | 8.05 | 11.69 | 8.00 | 12.26 | 7.68 | 11.73 |
| stem | rind | top | | | | | | |
| stem | rind | middle | | | | | | |
| stem | rind | bottom | | | | | | |
| stem | pith | top | | | | | | |
| stem | pith | middle | | | | | | |
| stem | pith | bottom | | | | | | |
| stem | rind | all | | | | | | |
| stem | rind | all | | | | | | |
| stem | rind | all | | | | | | |
| stem | pith | all | | | | | | |
| stem | pith | all | | | | | | |
| stem | pith | all | | | | | | |

### Example 4

### Production of DNA Elements for RNAi Vectors

Three fragments from the SbCSE cDNA transcript are used in three different RNAi constructs. The three fragments are localized (1) in the 5′ portion of the coding region (SEQ ID NO:11), (2) the central portion of the open reading frame (SEQ ID NO:12), and (3) the 3′ portion of the open reading frame (SEQ ID NO:13), respectively as shown in Table 15 above. The RNAi cassette for target DNA sequences (including the necessary restriction enzyme sites at the ends of the synthesized DNA fragments) are synthesized and shown in Table 19 (SEQ ID NOs:14-19). Either the maize Ubiquitin promoter (ZmUbi) and Arabidopsis terminator (AtT6) or sorghum CSE promoter (upstream 2 kb) and Arabidopsis terminator (AtT6) or SbCSE terminator (Sb-CSE) are synthesized and cloned into the pUC57 vector. Each synthesized RNAi cassette is cloned into a promoter terminator vector backbone. The silencing constructs shown in Table 19 can produce hairpin RNA (hpRNA) of the target gene for gene silencing. The constructs comprise an inverted repeat separated by a homologous spacer; the promoter of the Version 1 silencing construct is immediately operably linked to a shorter sense sequence. The part of the longer sense section is the loop part of hpRNAs when transcribed. The Version 2 silencing construct consists of a promoter that is immediately operably linked to a shorter antisense section, a longer sense section complementary to the 5′ end of the shorter antisense section, wherein the 5′ end of the longer sense section forms an intervening loop. The promoter and terminator elements with the correct restriction sites (Table 20, SEQ ID NOs:20 and 21) are then amplified using PCR from PUC57 vector., following the same PCR conditions as described above. All PCR products and digested vector fragments are purified from a 1% TAE/agarose gel using the QIAquick Gel Extraction Kit (Qiagen, Germantown, Md.).

Alternatively, the SbCSE promoter sequence (SEQ ID NO:60) can be used to target RNAi expression of genes in cells that express endogenous SbSCE RNA transcript to achieve efficient RNAi based gene silencing. The 700 bp of the 3′ UTR of SbCSE gene (SEQ ID NO:61) can be used as the terminator. SEQ ID NOs:62 and 63 are shown in Table 21.

TABLE 19
RNAi cassettes

| | | SEQ ID | | |
|---|---|---|---|---|
| target SEQ ID NO | | NO: | Sequence | |
| sbCSE: | Version 1 | 14 | gagctcggcg cgccccaacc aaccccacca cgccaacgtc cgggaccaaa ctctgatccc | 60 |
| 5′UTR + | | | caccatgcag gcggacgggg acgcgccggc gccggcgccg gccgtccact tctggggcga | 120 |
| 5′CDS | | | gcacccggcc acggaggcgg agttctacgc ggcgcacggc gcggagggcg agccctccta | 180 |
| | | | cttcaccacg cccgacgcgg gcgcccggcg gctcttcacg cgcgcgtgga ggccccgcgc | 240 |
| | | | gcccgagcgg cccaggccgc gaagcaggcg aacccggacc gcgcgaggaa gaccgccgtg | 300 |
| | | | gactggaacg tccagctgac gtcgttgccg tagccgtgga ccatgaagac gagcgccctg | 360 |
| | | | ggccgctcgg gcgcgcgggg cctccacgcg cgcgtgaaga gccgccgggc gcccgcgtcg | 420 |
| | | | ggcgtggtga agtaggaggg ctcgccctcc gcgccgtgcg ccgcgtagaa ctccgcctcc | 480 |
| | | | gtggccgggt gctcgcccca gaagtggacg gccggcgccg gcgccggcgc gtccccgtcc | 540 |
| | | | gcctgcatgg tggggatcag agtttggtcc cggacgttgg cgtggtgggg ttggttggat | 600 |
| | | | ttaaatggta cc | 612 |
| | Version 2 | 15 | gagctcggcg cgcccctggg ccgctcgggc gcgcggggcc tccacgcgcg cgtgaagagc | 60 |
| | | | cgccgggcgc ccgcgtcggg cgtggtgaag taggagggct cgccctccgc gccgtgcgcc | 120 |
| | | | gcgtagaact ccgcctccgt ggccgggtgc tcgccccaga agtggacggc cggcgccggc | 180 |
| | | | gccggcgcgt ccccgtccgc ctgcatggtg gggatcagag tttggtcccg gacgttggcg | 240 |
| | | | tggtggggtt ggttggtgcc ccgtcgcaac tggcagcagc agcgaccagc gactccccca | 300 |
| | | | actcgccggc caccagtagt tccctgcttc cccatcccat ccacacacac cgcacaccaa | 360 |
| | | | ccaaccccac cacgccaacg tccgggacca aactctgatc cccaccatgc aggcggacgg | 420 |
| | | | ggacgcgccg gcgccggcgc cggccgtcca cttctggggc gagcacccgg ccacggaggc | 480 |
| | | | ggagttctac gcggcgcacg gcgcggaggg cgagccctcc tacttcacca cgcccgacgc | 540 |
| | | | gggcgcccgg cggctcttca cgcgcgcgtg gaggccccgc gcgcccgagc ggcccaggat | 600 |
| | | | ttaaatggta cc | 612 |
| sbCSE: | Version 1 | 16 | gagctcggcg cgccgggcgc tcgtcttcat ggtccacggc tacggcaacg acgtcagctg | 60 |
| CDS | | | gacgttccag tccacggcgg tcttcctcgc gcggtccggg ttcgcctgct tcgcggccga | 120 |
| | | | cctcccgggc cacggccgct cccacggcct ccgcgccttc gtgcccgacc tcgacgccgc | 180 |
| | | | cgtcgccgac ctcctcgcct tcttccgcgc cgtcagggcg agggaggagc acgcgggcct | 240 |
| | | | gccctgcttc ctcttcgggg agtcccggtc ggagatcctg cacatgggcg cgacgaggac | 300 |
| | | | cgcccccgcc cactcctccg gccgcgtgcg gaggtggatg agcaggcaga tggccccgcc | 360 |
| | | | catggactcc ccgaagagga agcagggcag gcccgcgtgc tcctccctcg ccctgacggc | 420 |
| | | | gcggaagaag gcgaggaggt cggcgacggc ggcgtcgagg tcgggcacga aggcgcggag | 480 |
| | | | gccgtgggag cggccgtggc ccgggaggtc ggccgcgaag caggcgaacc cggaccgcgc | 540 |
| | | | gaggaagacc gccgtggact ggaacgtcca gctgacgtcg ttgccgtagc cgtggaccat | 600 |
| | | | gaagacgagc gcccatttaa atggtacc | 628 |
| | Version 2 | 17 | gagctcggcg cgccgactcc ccgaagagga agcagggcag gcccgcgtgc tcctccctcg | 60 |
| | | | ccctgacggc gcggaagaag gcgaggaggt cggcgacggc ggcgtcgagg tcgggcacga | 120 |
| | | | aggcgcggag gccgtgggag cggccgtggc ccgggaggtc ggccgcgaag caggcgaacc | 180 |
| | | | cggaccgcgc gaggaagacc gccgtggact ggaacgtcca gctgacgtcg ttgccgtagc | 240 |
| | | | cgtggaccat gaagacgagc gccccacggc gcggagggcg agccctccta cttcaccacg | 300 |
| | | | cccgacgcgg gcgcccggcg gctcttcacg cgcgcgtgga ggccccgcgc gcccgagcgg | 360 |
| | | | cccagggcgc tcgtcttcat ggtccacggc tacggcaacg acgtcagctg gacgttccag | 420 |
| | | | tccacggcgg tcttcctcgc gcggtccggg ttcgcctgct tcgcggccga cctcccgggc | 480 |
| | | | cacggccgct cccacggcct ccgcgccttc gtgcccgacc tcgacgccgc cgtcgccgac | 540 |
| | | | ctcctcgcct tcttccgcgc cgtcagggcg agggaggagc acgcgggcct gccctgcttc | 600 |
| | | | ctcttcgggg agtcatttaa atggtacc | 628 |
| sbCSE: | Version 1 | 18 | gagctcggcg cgccatcgag cgtgtccgcg gcgacatcct ggcctggctc aacgagagat | 60 |
| 3′CDS + | | | gcacaccgcc ggcaactccc tggcaccgtg acatacctgt cgaataagca ttccaggctg | 120 |
| 3′UTR | | | ttcagattcc gatgtatcga ttacacaaga aaattggttt catgtacaac gattcttata | 180 |
| | | | ctatacgcta tatacttggt cgtattttat tatcgacccc aagcatttgc agcattcttt | 240 |
| | | | tacactgatc aggcaaccaa cattttgtat atccaagcca ctaaacctga ccagacagtt | 300 |
| | | | tatagtcaaa tacgaccaag tatatagcgt atagtataag aatcgttgta catgaaacca | 360 |
| | | | attttcttgt gtaatcgata catcggaatc tgaacagcct ggaatgctta ttcgacaggt | 420 |
| | | | atgtcacggt gccagggagt tgccggcggt gtgcatctct cgttgagcca ggccaggatg | 480 |
| | | | tcgccgcgga cacgctcgat atttaaatgg taccctcgat | 520 |
| | Version 2 | 19 | gagctcggcg cgccaaatac gaccaagtat atagcgtata gtataagaat cgttgtacat | 60 |
| | | | gaaaccaatt ttcttgtgta atcgatacat cggaatctga acagcctgga atgcttattc | 120 |
| | | | gacaggtatg tcacggtgcc agggagttgc cggcggtgtg catctctcgt tgagccaggc | 180 |
| | | | caggatgtcg ccgcggacac gctcgattca gccgcgccct gtacgccgcc gccgccagca | 240 |
| | | | aggacaagac tatcaagata tacgacggga tgctccactc cttgctattt ggggaaccgg | 300 |
| | | | acgagaaatc gagcgtgtcc gcggcgacat cctggcctgg ctcaacgaga gatgcacacc | 360 |
| | | | gccggcaact ccctggcacc gtgacatacc tgtcgaataa gcattccagg ctgttcagat | 420 |
| | | | tccgatgtat cgattacaca agaaaattgg tttcatgtac aacgattctt atactatacg | 480 |
| | | | ctatatactt ggtcgtattt atttaaatgg tacc | 514 |

TABLE 20
Promoter and terminator backbone with restriction enzyme sites
(PacI BamHI ... promoter ... SacI ... KpnI ... terminator ... BgIIIPacI)

| SEQ ID NO: | Sequence | |
|---|---|---|
| 20 (ZmUbi and | gaattcttaa ttaaggatcc gtgcagcgtg acccggtcgt gcccctctct agagataatg | 60 |
| AtT6 terminator) | agcattgcat gtctaagtta taaaaaatta ccacatattt tttttgtcac acttgtttga | 120 |
| | agtgcagttt atctatcttt atacatatat ttaaacttta ctctacgaat aatataatct | 180 |
| | atagtactac aataatatca gtgttttaga gaatcatata aatgaacagt tagacatggt | 240 |
| | ctaaaggaca attgtatttt gacaacagga ctctacagtt ttatcttttt agtgtgcatg | 300 |
| | tgttctcctt tttttttgca aatagcttca cctatataat acttcatcca ttttattagt | 360 |
| | acatccattt agggtttagg gttaatggtt tttatagact aattttttta gtacatctat | 420 |
| | tttattctat tttagcctct aaattaagaa aactaaaact ctattttagt ttttttattt | 480 |
| | aatagtttag atataaaata gaataaaata aagtgactaa aaattaaaca aatacccttt | 540 |
| | aagaaattaa aaaaactaag gaaacatttt tcttgtttcg agtagataat gccagcctgt | 600 |
| | taaacgccgt cgacgagtct aacggacacc aaccagcgaa ccagcagcgt cgcgtcgggc | 660 |
| | caagcgaagc agacggcacg gcatctctgt cgctgcctct ggacccctct cgagagttcc | 720 |
| | gctccaccgt tggacttgct ccgctgtcgg catccagaaa ttgcgtggcg gagcggcaga | 780 |
| | cgtgagccgg cacggcaggc ggcctcctcc tcctctcacg gcaccggcag ctacggggga | 840 |
| | ttcctttccc accgctcctt cgctttccct tcctcgcccg ccgtaataaa tagacacccc | 900 |
| | ctccacaccc tctttcccca acctcgtgtt gttcggagcg cacacacaca caaccagatc | 960 |
| | acccccaaat ccacccgtcg gcacctccgc ttcaaggtac gccgctcgtc ctcccccccc | 1020 |
| | ccccccctct ctaccttctc tagatcggcg ttccggtcca tgcatggtta gggcccggta | 1080 |
| | gttctacttc tgttcatgtt tgtgttagat ccgtgtttgt gttagatccg tgctgctagc | 1140 |
| | gttcgtacac ggatgcgacc tgtacgtcag acacgttctg attgctaact tgccagtgtt | 1200 |
| | tctctttggg gaatcctggg atggctctag ccgttccgca gacgggatcg atttcatgat | 1260 |
| | tttttttgtt tcgttgcata gggtttggtt tgcccttttc ctttatttca atatatgccg | 1320 |
| | tgcacttgtt tgtcgggtca tcttttcatg cttttttttg tcttggttgt gatgatgtgg | 1380 |
| | tctggttggg cggtcgttct agatcggagt agtattctgt ttcaaactac ctggtggatt | 1440 |
| | tattaatttt ggatctgtat gtgtgtgcca tacatattca tagttacgaa ttgaagatga | 1500 |
| | tggatggaaa tatcgatcta ggataggtat acatgttgat gcgggtttta ctgatgcata | 1560 |
| | tacagagatg ctttttgttc gcttggttgt gatgatgtgg tgtggttggg cggtcgttca | 1620 |
| | ttcgttctag atcggagtag aatactgttt caaactacct ggtgtattta ttaattttgg | 1680 |
| | aactgtatgt gtgtgtcata catcttcata gttacgagtt taagatggat ggaaatatcg | 1740 |
| | atctaggata ggtatacatg ttgatgtggg ttttactgat gcatatacat gatggcatat | 1800 |
| | gcagcatcta ttcatatgct ctaaccttga gtacctatct attataataa acaagtatgt | 1860 |
| | tttataatta tttcgatctt gatatacttg gatgatggca tatgcagcag ctatatgtgg | 1920 |
| | atttttttag ccctgccttc atacgctatt tatttgcttg gtactgtttc ttttgtcgat | 1980 |
| | gctcaccctg ttgtttggtg ttacttctgc aggagctcgc taccttaaga gaggtttaaa | 2040 |
| | cggtaccctt ttaagatggg atgtctttaa tatgtagaac ctcgtttttg gttataattt | 2100 |
| | tcgttgcatg tctctcttct cttgtactat tcacacttgt tgtttgctgt atcttcttct | 2160 |
| | tcagtttgct ttgctacgat tgtggttttt ggagacatta tagctcatta actgtttgtg | 2220 |
| | agaccaaatg tgtcagaatc cgctattaca cacctagttg tcaacattca ctacaaataa | 2280 |
| | tatggacttt aacgtcggtt taaggcatcc aataaaactg acgttatgtt tctctttcct | 2340 |
| | cgttttgtcg accaaaaaaa ctgaccctaa atgtagatct ttaattaaaa gctt | 2394 |
| 21 (sbCSE | gaattcttaa ttaaggatcc aaaattatgg ctaaaagtat tgtttactga tttattatgg | 60 |
| upstream 2kb and | aagaaaagca ctactgacta gcagaaaaag tacggcttat aacacaaacg aacggaacct | 120 |
| AtT6 terminator) | atgtactaac tattaactag atcggtgcta aaatgtactc cctccattcc taaataaatt | 180 |
| | aaattctaga gttatcttaa ataaaacttt tttaacgttt tactgaattt atagaaagaa | 240 |
| | acacaaatat ttatgacacc aaatgatcat attataaaaa ttattatggt gtatctcatg | 300 |
| | atactaatat agtgtcataa attttgacat ttttattaaa taaaataaaa tttagtcaaa | 360 |
| | ttttaaaaag ttggacttaa ggcaaatcta aaagttgatt tattcaggaa tcagaggaag | 420 |
| | ttaaaaaaaa atgattccag agctgttctt aaatttgttg caaacacatg gagggattgc | 480 |
| | ttaaagatac atgggctcag gggatgctgc agtaccggta gcacctgccc tgagctggcg | 540 |
| | gacaactaaa atatttaagc aaaaaaaatg atggctacga ttgtaaattg agcgtagttc | 600 |
| | agcaagtgaa cccaatccac catgttcaaa tttttctatc ttttttctag aatttaacaa | 660 |
| | cgttgtgttt tttaatgtta ggagacatgg tactatgatc aactgatcat ttcgttaacc | 720 |
| | tttttatgta cagcatcatc gagcatgcac tggtccgaga tataggcagc ttaagcacca | 780 |
| | gttttatgtg cagccggata ggtgatatgt ccttgctaat taggctccta tttgtagcta | 840 |
| | tagtattatc tattcatacg gccctatcca ttgctaagag caagtataat aagttatttt | 900 |
| | tagccggttg caagagtcca cctaatcaaa aaagcagacc acgtaggaga gatattaggg | 960 |
| | cactcacaat gcaagactct atcacaaagt ccaagacaat taattacata ttatttatgg | 1020 |
| | tattttgctg atgtggcagc atatttattg aagaaagagg tagaaaaaaa taagactcca | 1080 |
| | agtcttattt agactctaag tccacattgt tcgaggtaat aaataacttt agactctatg | 1140 |
| | atagagtctg cattgtgagt gcccttatag agccggcgat tcccatctcg cccgcctcta | 1200 |
| | gctcaagata cgagaaaaaa aaatttgtcc tagacgtctt ccagcccgct gtgagcgcga | 1260 |
| | tgccgacgct tccatctccc gccgttccgc tccctaattc tgtgctctac tcgatcatta | 1320 |
| | cctgacatta aatacttgta tttttattat agtacacctc caagctggct aaaccatttt | 1380 |
| | gatgtttagg ttagtacatg ttgatgttta ggttaggtgt aagtgatatg acaacttctc | 1440 |
| | tcaaccgtca gccggctaaa ccattagcct tgctctaact gggctttatt tgttgctaca | 1500 |
| | gtactagtat ctacaccttc ggtcgtaccc attttcacac tctatgaaaa cgctccgttt | 1560 |
| | aatggaactt gttttctgct taatctgcca aggctctcgt tcatcaaaag aaaataaagc | 1620 |
| | gagaatcagg tgatggagcg acatggttct taaaatcatt tttttcataa actaaaaatc | 1680 |
| | gaaaggttta ttggccctaa taatgtcggt acacgagtta atgttccctg catgggccaa | 1740 |
| | ctatgaacga gaatagtata ccacgtggac ccgtgggccg cggcacgagc cgttccacct | 1800 |
| | acccgcaacg aaccgagcga tttcgccgtc ccgcatccaa acgcccccag cagcccttcc | 1860 |
| | cctgccccag tgccccgtcg caactggcag cagcagcgac cagcgactcc cccaactcgc | 1920 |
| | cggccaccag tagttccctg cttccccatc ccatccacac acaccgcaca ccaaccaacc | 1980 |
| | ccaccacgcc aacgtccggg accaaactct gatccccacc ggagctcgct accttaagag | 2040 |
| | aggtttaaac ggtacccttt taagatggga tgtctttaat atgtagaacc tcgtttttgg | 2100 |
| | ttataatttt cgttgcatgt ctctcttctc ttgtactatt cacacttgtt gtttgctgta | 2160 |
| | tcttcttctt cagtttgctt tgctacgatt gtggtttttg gagacattat agctcattaa | 2220 |
| | ctgtttgtga gaccaaatgt gtcagaatcc gctattacac acctagttgt caacattcac | 2280 |
| | tacaaataat atggacttta acgtcggttt aaggcatcca ataaaactga cgttatgttt | 2340 |
| | ctctttcctc gttttgtcga ccaaaaaaac tgaccctaaa tgtagatctt taattaaaag | 2400 |
| | ctt | 2403 |

TABLE 21
Alternative sequences

| SEQ ID NO: | Sequence | |
|---|---|---|
| 62 (SbCSE | gaattcttaa ttaaggatcc aaaattatgg ctaaaagtat tgtttactga tttattatgg | 60 |
| promoter) | aagaaaagca ctactgacta gcagaaaaag tacggcttat aacacaaacg aacggaacct | 120 |
| | atgtactaac tattaactag atcggtgcta aaatgtactc cctccattcc taaataaatt | 180 |
| | aaattctaga gttatcttaa ataaaacttt tttaacgttt tactgaattt atagaaagaa | 240 |
| | acacaaatat ttatgacacc aaatgatcat attataaaaa ttattatggt gtatctcatg | 300 |
| | atactaatat agtgtcataa attttgacat ttttattaaa taaaataaaa tttagtcaaa | 360 |
| | ttttaaaaag ttggacttaa ggcaaatcta aaagttgatt tattcaggaa tcagaggaag | 420 |
| | ttaaaaaaaa atgattccag agctgttctt aaatttgttg caaacacatg gagggattgc | 480 |
| | ttaaagatac atgggctcag gggatgctgc agtaccggta gcacctgccc tgagctggcg | 540 |
| | gacaactaaa atatttaagc aaaaaaaatg atggctacga ttgtaaattg agcgtagttc | 600 |
| | agcaagtgaa cccaatccac catgttcaaa tttttctatc ttttttctag aatttaacaa | 660 |
| | cgttgtgttt tttaatgtta ggagacatgg tactatgatc aactgatcat ttcgttaacc | 720 |
| | tttttatgta cagcatcatc gagcatgcac tggtccgaga tataggcagc ttaagcacca | 780 |
| | gttttatgtg cagccggata ggtgatatgt ccttgctaat taggctccta tttgtagcta | 840 |
| | tagtattatc tattcatacg gccctatcca ttgctaagag caagtataat aagttatttt | 900 |
| | tagccggttg caagagtcca cctaatcaaa aaagcagacc acgtaggaga gatattaggg | 960 |
| | cactcacaat gcaagactct atcacaaagt ccaagacaat taattacata ttatttatgg | 1020 |
| | tattttgctg atgtggcagc atatttattg aagaaagagg tagaaaaaaa taagactcca | 1080 |
| | agtcttattt agactctaag tccacattgt tcgaggtaat aaataacttt agactctatg | 1140 |
| | atagagtctg cattgtgagt gcccttatag agccggcgat tcccatctcg cccgcctcta | 1200 |
| | gctcaagata cgagaaaaaa aaatttgtcc tagacgtctt ccagcccgct gtgagcgcga | 1260 |
| | tgccgacgct tccatctccc gccgttccgc tccctaattc tgtgctctac tcgatcatta | 1320 |
| | cctgacatta aatacttgta tttttattat agtacacctc caagctggct aaaccatttt | 1380 |
| | gatgtttagg ttagtacatg ttgatgttta ggttaggtgt aagtgatatg acaacttctc | 1440 |
| | tcaaccgtca gccggctaaa ccattagcct tgctctaact gggctttatt tgttgctaca | 1500 |
| | gtactagtat ctacaccttc ggtcgtaccc attttcacac tctatgaaaa cgctccgttt | 1560 |
| | aatggaactt gttttctgct taatctgcca aggctctcgt tcatcaaaag aaaataaagc | 1620 |
| | gagaatcagg tgatggagcg acatggttct taaaatcatt tttttcataa actaaaaatc | 1680 |
| | gaaaggttta ttggccctaa taatgtcggt acacgagtta atgttccctg catgggccaa | 1740 |
| | ctatgaacga gaatagtata ccacgtggac ccgtgggccg cggcacgagc cgttccacct | 1800 |
| | acccgcaacg aaccgagcga tttcgccgtc ccgcatccaa acgcccccag cagcccttcc | 1860 |
| | cctgccccag tgccccgtcg caactggcag cagcagcgac cagcgactcc cccaactcgc | 1920 |
| | cggccaccag tagttccctg cttccccatc ccatccacac acaccgcaca ccaaccaacc | 1980 |
| | ccaccacgcc aacgtccggg accaaactct gatccccacc | 2020 |
| 61 (SbCSE 3′ | gcattccagg ctgttcagat tccgatgtat cgattacaca agaaaattgg tttcatgtac | 60 |
| for terminator | aacgattctt atactatacg ctatatactt ggtcgtattt gactataaac tgtctggtca | 120 |
| with promoter) | ggtttagtgg cttggatata caaaatgttg gttgcctgat cagtgtaaaa gaatgctgca | 180 |
| | aatgcttggg gtcgataata tcagctctct tcgggggcta ttgatggcag cacaaggcgt | 240 |
| | tccctgcctt gtacaagctt ggcagaacga attttatccc cggtcttaat ctgcgataga | 300 |
| | acatctcttc catccgtggt atacctgcaa ttgtttggat atacgcataa catttcttac | 360 |
| | agcgttctta tccacaatgg aatagatcga ttttgcaact caatgtttac ataatgaaat | 420 |
| | cagtcacgac ttacccgaaa actgaaaact gtccctcatc aaacgatatt cctcctaagc | 480 |
| | cagactacag aaaagaaaga gaaacatgtt aactcacata tctatacaga aattcatgct | 540 |
| | tcttcagatt attacaggct ggagaagcaa cttgttactt gttatattag tacattgggc | 600 |
| | attcatattc tttgtatgac tgacctggca gagtctggtc tgttatctga atacttatat | 660 |
| | tcatctttat gtttaaagaa aagcaaatat ggttt | 695 |

### Example 5

### Vector Construction

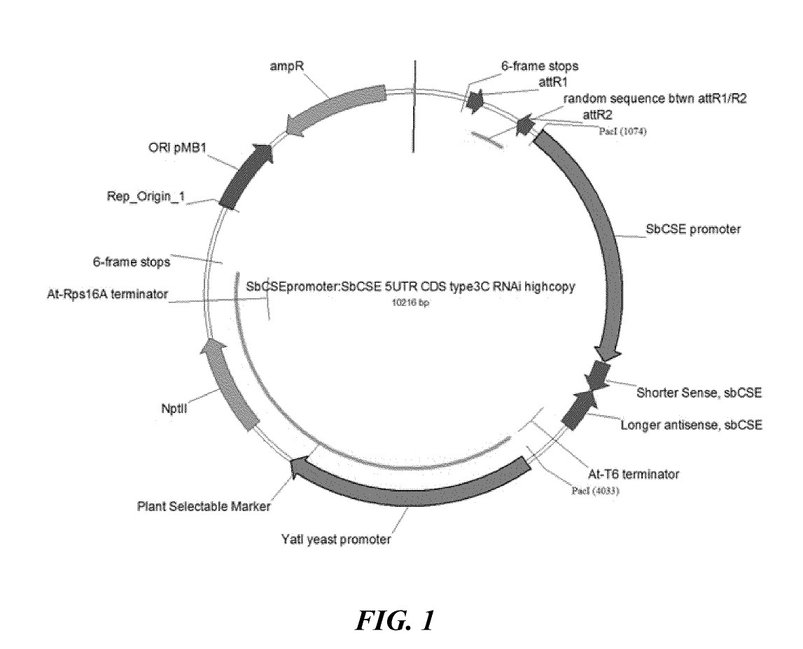

The RNAi vector is created in order to incorporate the desired DNA elements for the SbCSE RNAi experiment (FIG. 1). The antisense and sense DNA elements including the necessary restriction enzyme sites at the ends of the synthesized DNA fragments are synthesized and cloned into the multi cloning site of pUC57 (shown in Table 11). The maize Ubiquitin promoter (Zm-Ubi v3) and Arabidopsis terminator (At-T6 v1) or sorghum CSE promoter (upstream 2 kb) (Sb-CSE v1) and Arabidopsis terminator (At-T6 v1) are synthesized as shown in Table 12. The promoter and terminator element (P/T) cassettes start with Pacl and end with Pacl. Between the promoter and terminator are four restriction enzyme sites: Sacl, Ascl, Swal and Kpnl. High throughput vector system (HTPV) containing multiple cloning sites and a plant selective marker (for example, the Yatl promoter driving expression of the Nptll gene for Geneticin® (Life Technologies) resistance) are synthesized. The synthesized antisense/sense fragments are digested with Sacl and Kpnl and cloned into the synthesized P/T vector that has been digested with Sacl and Kpnl and treated with alkaline phosphatase. Then the RNAi cassettes are built in the HTPV vector by digesting the RNAi cassettes out of the P/T vector with Pacl and inserting into the HTPV vector that has been digested with Pacl and treated alkaline phosphatase. The complete vectors are confirmed by sequencing.

### Example 6

### Production of Transgenic Sorghum with Down-Regulated SbCSE Expression

In order to obtain transgenic plants with down-regulated SbCSE, sorghum is transformed with the SbCSE RNAi vectors described in Example 5. In addition to transforming sorghum with the SbCSE RNAi vectors, we will also transform control plants with the base vector, pHan-OsAct-T6. We will use either particle bombardment (and co-bombard the pHan-SbCSE vectors with a second plasmid containing the plant selection cassette YatI:NptII:AtT6) or Agrobacterium-mediated transformation (after subcloning the RNAi cassettes into a binary vector suitable for Agrobacterium-mediated transformation) to introduce the RNAi vector DNA into the genome of wild-type sorghum. Potentially transformed events will be cultured under Geneticin selection consisting of 20 mg/L G418 for two weeks, then 40 mg/L G418 for two weeks, and finally 60 mg/L G418 for a further two weeks. Resistance to this antibiotic is conferred by the plant selectable marker that will be co-bombarded with pHan-SbCSE-5′/C/3′ plasmids, so any untransformed tissue should be killed on the selective agar plates. Selective pressure will be maintained through the stages of regeneration and rooting to ensure a minimum number of escapes. Regenerated callus and subsequent plants will be screened for the RNAi cassette by PCR using the primers of SEQ ID NOs found in Table 22. The same DNA extraction and PCR techniques described in Example 1.3 will be used for screening the transgenic events.

TABLE 22
Event screening primers

| | | | | |
|---|---|---|---|---|
| SEQ | | | | |
| ID NO | Target | type | primer | seq |

ZmUbi:SbCSE

| | | | | |
|---|---|---|---|---|
| 22 | 5′ | 3C | F | tctaacggac accaaccagc 20 |
| 23 | UTR + | | R | ctgcatggtg gggatcagag 20 |
| 24 | CDS | 3D | F | tctaacggac accaaccagc 20 |
| 25 | | | R | cgggaccaaa ctctgatccc 20 |
| 26 | CDS | 3C | F | tctaacggac accaaccagc 20 |
| 27 | | | R | ccgtggacca tgaagacgag 20 |
| 28 | | 3D | F | tctaacggac accaaccagc 20 |
| 29 | | | R | ctcgtcttca tggtccacgg 20 |
| 30 | CDS + | 3C | F | tctaacggac accaaccagc 20 |
| 31 | 3′ | | R | tgcatctctc gttgagccag 20 |
| 32 | UTR | 3D | F | tctaacggac accaaccagc 20 |
| 33 | | | R | ccaggctgtt cagattccga 20 |

sbCSE promoter:SbCSE

| | | | | |
|---|---|---|---|---|
| 34 | 5′ | 3C | F | ctgagctggc ggacaactaa 20 |
| 35 | UTR + | | R | gtggtgaagt aggagggctc 20 |
| 36 | CDS | 3D | F | ctgagctggc ggacaactaa 20 |
| 37 | | | R | gagccctcct acttcaccac 20 |
| 38 | CDS | 3C | F | ctgagctggc ggacaactaa 20 |
| 39 | | | R | ccgtggacca tgaagacgag 20 |
| 40 | | 3D | F | ctgagctggc ggacaactaa 20 |
| 41 | | | R | ctcgtcttca tggtccacgg 20 |
| 42 | CDS + | 3C | F | ctgagctggc ggacaactaa 20 |
| 43 | 3′ | | R | tgcatctctc gttgagccag 20 |
| 44 | UTR | 3D | F | ctgagctggc ggacaactaa 20 |
| 45 | | | R | ccaggctgtt cagattccga 20 |

### Example 7

### Characterization of Transgenic Plants

After potential transgenic events have been screened for the RNAi cassette using the primers of SEQ ID NOs:22-45 (Table 21), they are transferred from selective in vitro culture to soil and maintained until maturity in a controlled environment. Throughout development, the T₀ lines of transgenic plants, including all three of the SbCSE RNAi lines and the control lines containing the empty base vector are constantly monitored for phenotypic differences. Based on the observations of Vanholme et al. (Vanholme et al., 2013), we expect to see phenotypic differences between the control and experimental plants during vegetative development, at least from knock-out constructs, including reduced height when compared to empty base vector plants.

In order to confirm that the transfected RNAi cassettes are functional in the transgenic plants, we will assay transcript abundance of SbCSE by RT-PCR in the RNAi and control lines. Various tissue types are harvested from developing and mature plants from both transgenic and control lines. We will include the following tissue types in the RT-PCR assay: developing leaves, mature leaves, mature stem, developing entire inflorescences, developing sessile florets, developing pedicellate florets, mature sessile florets, and mature pedicellate florets. RNA will be extracted from these tissues using the RNeasy® Plant Mini Kit (Qiagen®; Redwood City, Calif.; USA). Using the RNA as template, cDNA and subsequent RT-PCR products will be generated in a single step using the OneStep RT-PCR Kit (Qiagen®). The primers for the SbCSE RT-PCR product (Table 23) were designed to be specific to SbCSE and they were designed to span the first intron of SBCSE, thus preventing amplification from genomic DNA. Also, the primers were designed to amplify a region of the ORF of SbCSE that was not used as the RNAi target, in order to avoid any possible amplification from transcripts derived from the transgene from the pHan-SbCSE-ORF construct.

TABLE 23
SbCSE RT-PCR primers

| SEQ ID NO: | Sequence |
|---|---|
| 46 | ttcctcttcg gggagtccat 20 |
| 47 | tgcatctctc gttgagccag 20 |

Alternatively, antibodies that specifically bind the SbCSE polypeptide can be used to evaluate SbCSE gene expression and to determine the overall efficiency of the RNAi vector in the plant cell. Antibodies to SbCSE polypeptides may be obtained by immunization with purified SbCSE polypeptide or a fragment thereof, or with SbCSE peptides produced by biological or chemical synthesis. Suitable procedures for generating antibodies include those described in Hudson and Hay (Hudson and Hay, 1980).

Polyclonal antibodies directed toward a SbCSE polypeptide generally are produced in animals (e.g., rabbits or mice) by means of multiple subcutaneous or intraperitoneal injections of SbCSE polypeptide or SbCSE peptide and an adjuvant. After immunization, the animals are bled and the serum assayed for anti-SbCSE polypeptide antibody titer.

Monoclonal antibodies directed toward a SbCSE polypeptide are produced using any method which provides for the production of antibody molecules by continuous cell lines in culture. Examples of suitable methods for preparing monoclonal antibodies include the hybridism methods of Kohler et al. (Kohler and Milstein, 1975) and the human B-cell hybridism method (Kozbor et al., 1984; Schook, 1987).

### Example 8

### Determination of Lignin Content, Lignin Composition and Forage Digestibility

Transgenic sorghum or mutants characterized for low to negligible amounts of SbCSE RNA expression are analyzed initially for lignin content and quality using Maule (Guo et al., 2001) or Phloroglucinol staining (Nair et al., 2002). Further, the transgenic plants that show reduced level of lignin are further characterized for lignin content and composition by thioacidolysis (Rolando et al., 1992) or by derivatization followed by reductive cleavage (DFRC) method (Lu and Ralph, 1997). The biomass of SbCSE mutant or RNAi down-regulated SbCSE plants is tested for forage digestibility using in vitro dry matter digestibility (IVDMD) assay for forage digestibility (Vogel et al., 1999) and by simultaneous saccharification and fermentation (SSF) for conversion of cellulose to ethanol (Shahsavarani et al., 2013).

### Example 9

### Identification of CSE Orthologs

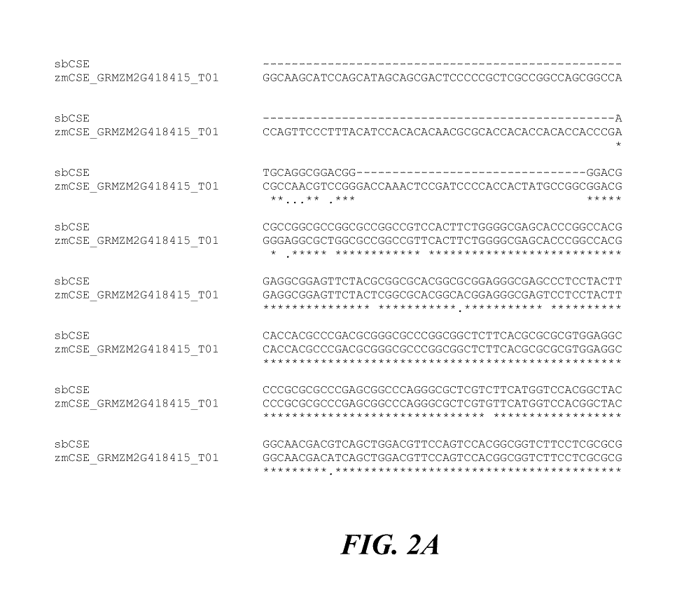

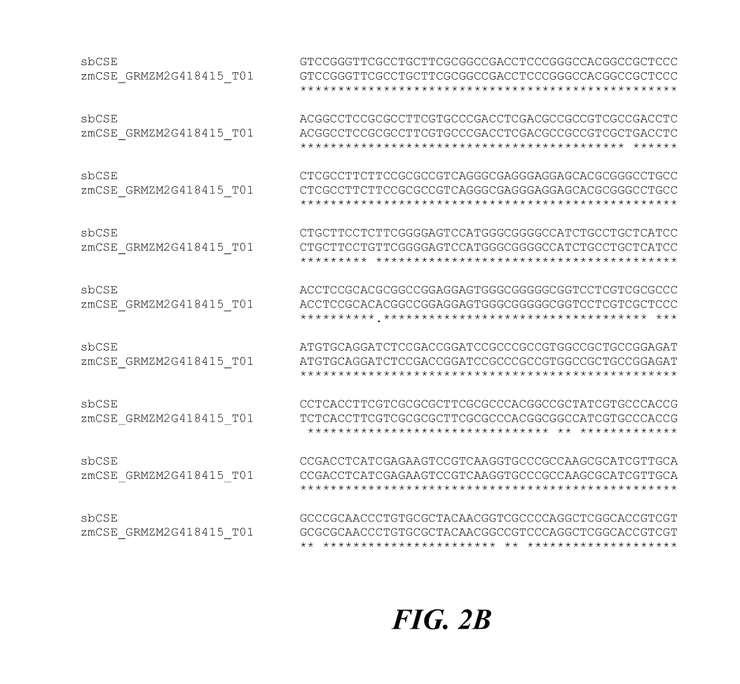

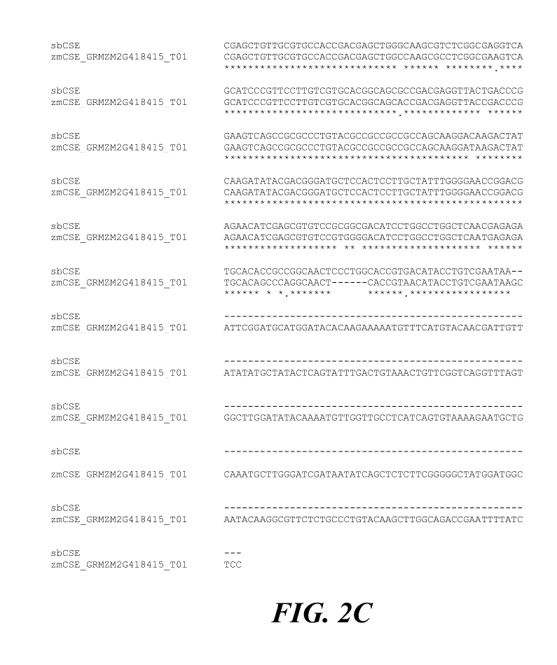

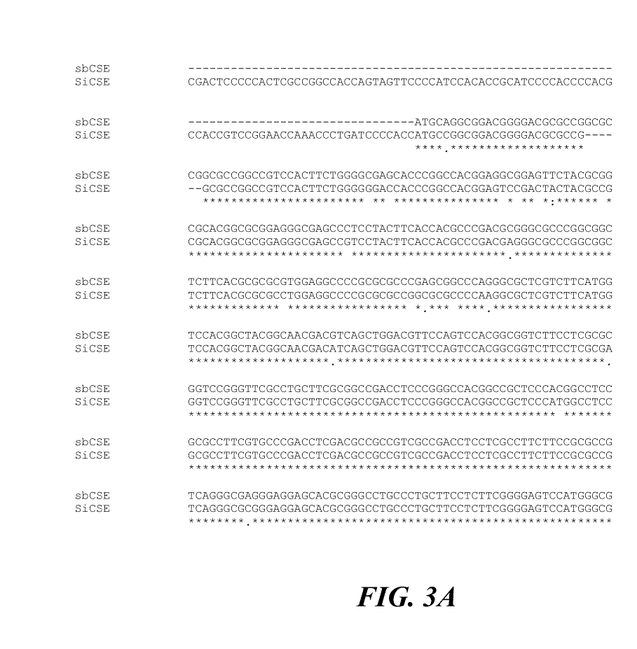

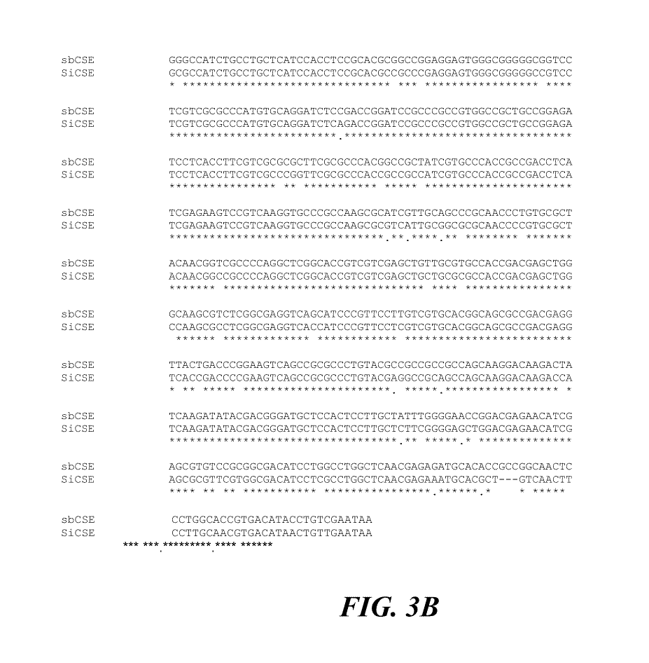

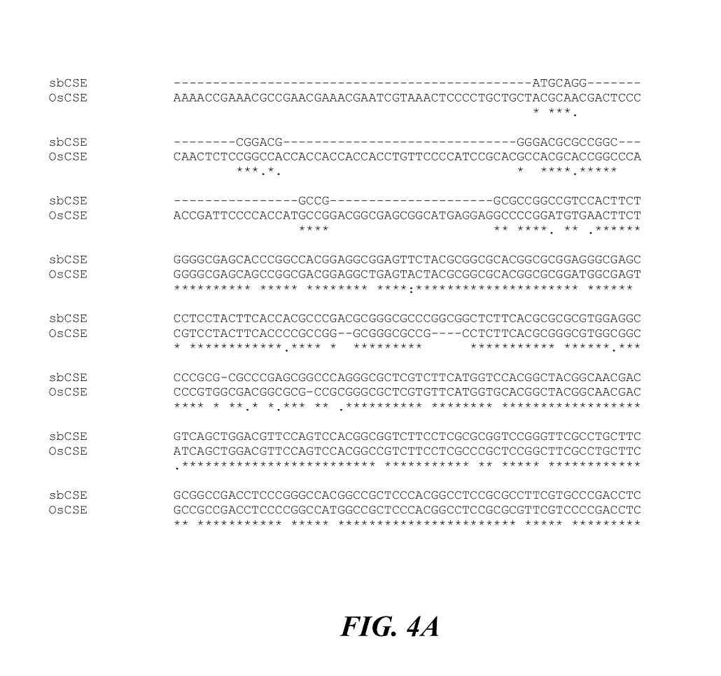

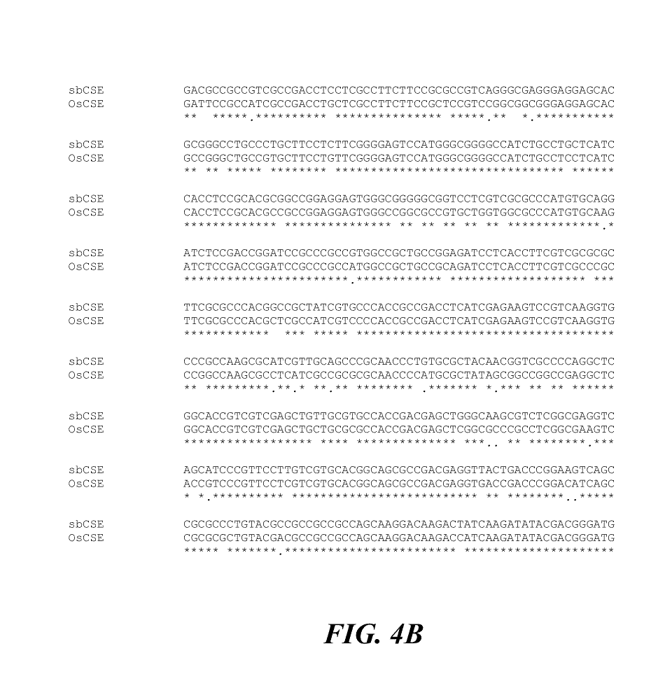

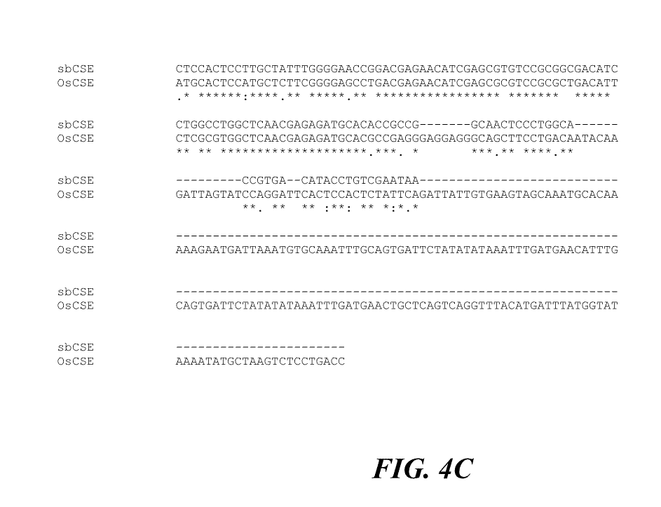

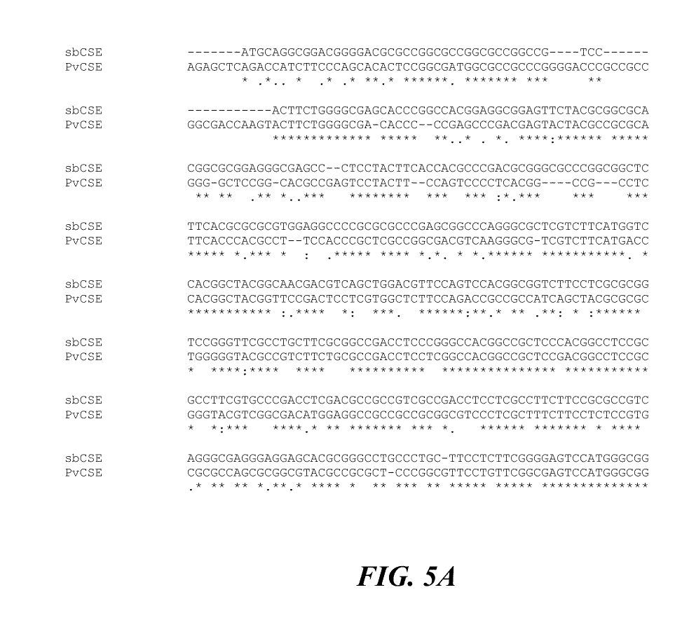

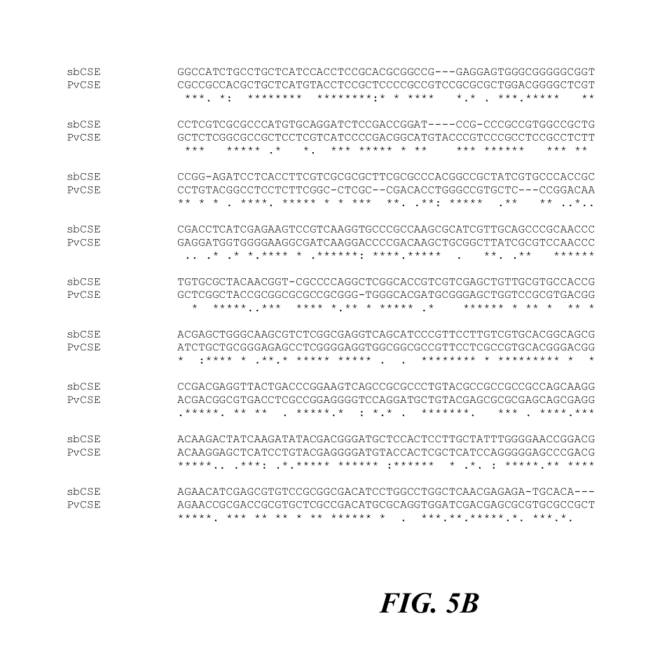

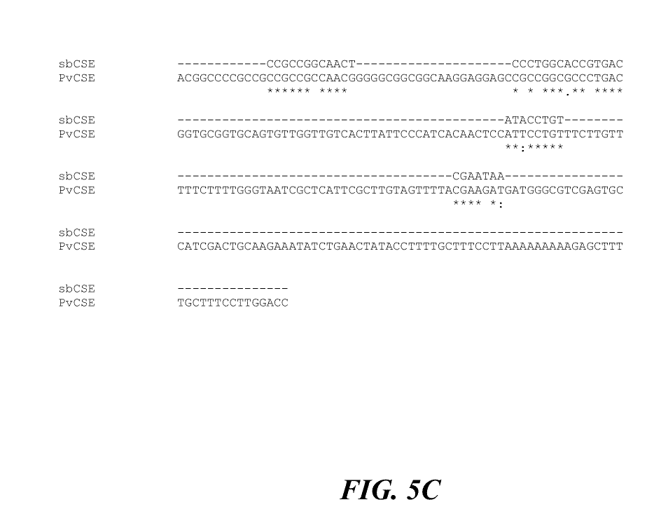

The amino acid sequence of the Arabidopsis CSE (SEQ ID NO:1) was used for identifying CSE orthologs in maize, foxtail millet (Setaria italica), rice, and switchgrass by BLAST search. The annotation sequences of maize, foxtail millet, rice and switchgrass were downloaded (via the Phytozome FTP site (Goodstein et al., 2012). The identified sequences (amino acid and nucleotide, the nucleotide showing the 5′ untranslated regions, the open reading frames, and the 3′ untranslated regions) are shown in Tables 7 and 8 (Z. mays; SEQ ID NOs:48 and 49), 9 and 10 (S. italica; SEQ ID NOs:50 and 51)), 11 and 12 (O. sativa; SEQ ID NOs: 52 and 53)), and 13 and 14 (P. virgatum; SEQ ID NOs:54 and 55). Sequence alignments using Clustal W (Larkin et al., 2007) of SbCSE (SEQ ID NO:6) with the identified sequences are shown in FIGS. 2A-2C (maize), 3A-3B (millet), 4A-4C (rice), and 5A-5C (switchgrass).

### Example 10

### Identification of Targeting RNAis from SbCSE Orthologs

Sequence alignment of sorghum CSE sequences with maize, foxtail millet, rice and switchgrass showed that the maize, setaria and rice sequences are highly conserved at nucleotide level (example 9). Thus the SbCSE ortholog sequences from maize, foxtail millet or rice could be used for generating RNAi constructs and for generating transgenic sorghum that are silenced for sorghum CSE gene. Sequence alignment was used to identify regions from maize, foxtail millet or rice that are highly homologous for designing RNAi sequences. DNA sequences from maize, foxtail millet or rice with regions of polynucleotides that are 100% identical and are more than 20-40 base pairs long were selected for designing the RNAi hairpin structures in the methods of the present technology. (Table 24 and Table 25).

TABLE 24
RNAi molecules of SbCSE orthologs

| SEQ | | | |
|---|---|---|---|
| ID NO: | Target | Sequence | |
| 56 | ZmCSE | gggcgctcgt gttcatggtc cacggctacg gcaacgacat cagctggacg ttccagtcca | 60 |
| | CDS | cggcggtctt cctcgcgcgg tccgggttcg cctgcttcgc ggccgacctc ccgggccacg | 120 |
| | | gccgctccca cggcctccgc gccttcgtgc ccgacctcga cgccgccgtc gctgacctcc | 180 |
| | | tcgccttctt ccgcgccgtc agggcgaggg aggagcacgc gggcctgccc tgcttcctgt | 240 |
| | | tcggggagtc | 250 |
| 57 | SiCSE | ggcgctcgtc ttcatggtcc acggctacgg caacgacatc agctggacgt tccagtccac | 60 |
| | CDS | ggcggtcttc ctcgcgaggt ccgggttcgc ctgcttcgcg gccgacctcc cgggccacgg | 120 |
| | | ccgctcccat ggcctccgcg ccttcgtgcc cgacctcgac gccgccgtcg ccgacctcct | 180 |
| | | cgccttcttc cgcgccgtca gggcgcggga ggagcacgcg ggcctgccct gcttcctctt | 240 |
| | | cggggagtcc | 250 |
| 58 | OsCSE | gcgcccatgt gcaagatctc cgaccggatc cgcccgccat ggccgctgcc gcagatcctc | 60 |
| | CDS | accttcgtcg cccgcttcgc gcccacgctc gccatcgtcc ccaccgccga cctcatcgag | 120 |
| | | aagtccgtca aggtgccggc caagcgc | 147 |

TABLE 25
RNAi cassettes of sbCSE orthologs

| target | SEQ ID | | |
|---|---|---|---|
| SEQ ID NO | NO: | Sequence | |
| ZmCSE | 59 | gagctcggcg cgcgggcgct cgtgttcatg gtccacggct acggcaacga catcagctgg | 60 |
| CDS | | acgttccagt ccacggcggt cttcctcgcg cggtccgggt tcgcctgctt cgcggccgac | 120 |
| | | ctcccgggcc acggccgctc ccacggcctc cgcgccttcg tgcccgacct cgacgccgcc | 180 |
| | | gtcgctgacc tcctcgcctt cttccgcgcc gtcagggcga gggaggagca cgcgggcctg | 240 |
| | | ccctgcttcc tgttcgggga gtcccggtcg gagatcctgc acatgggagc gacgaggacc | 300 |
| | | gcccccgccc actcctccgg ccgtgtgcgg aggtggatga gcaggcagat ggccccgccc | 360 |
| | | atggactccc cgaacaggaa gcagggcagg cccgcgtgct cctccctcgc cctgacggcg | 420 |
| | | cggaagaagg cgaggaggtc agcgacggcg gcgtcgaggt cgggcacgaa ggcgcggagg | 480 |
| | | ccgtgggagc ggccgtggcc cgggaggtcg gccgcgaagc aggcgaaccc ggaccgcgcg | 540 |
| | | aggaagaccg ccgtggactg gaacgtccag ctgatgtcgt tgccgtagcc gtggaccatg | 600 |
| | | aacacgagcg cccatttaaa tggtacc | 627 |
| SiCSE | 60 | gagctcggcg cgcggcgctc gtcttcatgg tccacggcta cggcaacgac atcagctgga | 60 |
| CDS | | cgttccagtc cacggcggtc ttcctcgcga ggtccgggtt cgcctgcttc gcggccgacc | 120 |
| | | tcccgggcca cggccgctcc catggcctcc gcgccttcgt gcccgacctc gacgccgccg | 180 |
| | | tcgccgacct cctcgccttc ttccgcgccg tcagggcgcg ggaggagcac gcgggcctgc | 240 |
| | | cctgcttcct cttcggggag tcctccggtc tgagatcctg cacatgggcg cgacgaggac | 300 |
| | | ggcccccgcc cactcctcgg gcggcgtgcg gaggtggatg agcaggcaga tggcgccgcc | 360 |
| | | catggactcc ccgaagagga agcagggcag gcccgcgtgc tcctcccgcg ccctgacggc | 420 |
| | | gcggaagaag gcgaggaggt cggcgacggc ggcgtcgagg tcgggcacga aggcgcggag | 480 |
| | | gccatgggag cggccgtggc ccgggaggtc ggccgcgaag caggcgaacc cggacctcgc | 540 |
| | | gaggaagacc gccgtggact ggaacgtcca gctgatgtcg ttgccgtagc cgtggaccat | 600 |
| | | gaagacgagc gccatttaaa tggtacc | 627 |
| OsCSE | 61 | gagctcggcg cgcgcgccca tgtgcaagat ctccgaccgg atccgcccgc catggccgct | 60 |
| CDS | | gccgcagatc ctcaccttcg tcgcccgctt cgcgcccacg ctcgccatcg tccccaccgc | 120 |
| | | cgacctcatc gagaagtccg tcaaggtgcc ggccaagcgc cgaggcgggc gccgagctcg | 180 |
| | | tcggtggcgc gcagcagctc gacgacggtg ccgagcctcg gccggccgct atagcgcatg | 240 |
| | | gggttgcgcg cggcgatgag gcgcttggcc ggcaccttga cggacttctc gatgaggtcg | 300 |
| | | gcggtgggga cgatggcgag cgtgggcgcg aagcgggcgacgaaggtgag gatctgcggc | 360 |
| | | agcggccatg gcgggcggat ccggtcggag atcttgcacatgggcgcatt taaatggtac | 420 |
| | | c | 421 |

TABLE OF SELECTED ABBREVIATIONS

| Abbreviation | Term |
|---|---|
| ADF | Acid detergent fiber |
| AHAS | Acetohydroxyacid synthase |
| amiRNA | Artificial microRNA |
| AP | Alkaline phosphatase |
| CAF | CARPEL FACTORY |
| CaMV | Cauliflower Mosaic Virus |
| CAT | Chloramphenicol acetyltransferase |
| CP | Crude protein |
| CSE | Caffeoyl shikimate esterase |
| DM | Dry matter |
| EE | Ether extract |
| GFP | Green fluorescent protein |
| GUS | Beta glucuronidase |
| hpRNA | Hairpin RNA |
| HRP | Horseradish peroxidase |
| LacZ | Beta galactosidase |
| LB | Left border |
| Luc | Luciferase |
| MS | Murashige and Skoog |
| NDF | Neutral detergent fiber |
| NEG | Net energy for gain |
| NEM | Net energy for maintenance |
| NOS | Nopaline synthase |
| OCS | Octopine synthase |
| PTGS | Post-transcriptional gene silencing |
| RB | Right border |
| RdRP | RNA-dependent RNA polymerase |
| RISC | RNA-induced silencing complex |
| RNAi | RNA interference |
| Sb | Sorghum bicolor |
| SbCSE | Sorghum caffeoyl shikimate esterase |
| siRNA | Small interfering RNA |
| TALENs | Transcription Activator-like Effector Nucleases |
| TDN | Total digestible nutrient |
| UTR | Untranslated region |
| VIGS | Virus-induced gene silencing |

### Targeted Mutagenesis for Generating Dominant Traits

The terms “dominant” and “recessive” traits describe the inheritance patterns of a certain phenotype to pass from parent to offspring. Sexually reproducing species such as plants, animals and human have two copies of each gene. The two copies, called alleles, can be slightly different from each other. The differences can cause variations in the protein that's produced, or they can change protein expression: when, where, and how much protein is made. These proteins can affect traits, so variations in protein activity or expression can produce different phenotypes.

A dominant allele produces a phenotype in individual organisms who have one copy of the allele, which can come from just one or both parents. For a recessive allele to produce a phenotype, the individual must have two copies, one from each parent. An individual organism with one dominant and one recessive allele for a gene will demonstrate the dominant phenotype. They are generally considered “carriers” of the recessive allele where the recessive phenotype is not expressed.

In commercial agriculture breeding where hybrid systems are used to produce improved yield and agronomic traits, dominant traits are preferred since it is easy to transfer the trait from one parent to another and select the trait in the progeny lines rapidly. For dominant traits with a visible phenotype, selection can be quick and efficient to identify those plants which carry the gene(s) of interest. A cross between a parent with homozygous dominant trait and a second parent with homozygous recessive trait will result in 100% of progeny plants expressing the dominant trait of interest. Even when the dominant trait is heterozygous, 50% of the progeny will exhibit the trait in the progeny and thus facilitate rapid selection.

In contrast, recessive traits are only expressed when the recessive genes are present in a homozygous state for both the alleles. Thus for commercial plant breeding, selection of recessive traits can be cumbersome since both parents need to be homozygous for the recessive alleles for the trait.

The difficulty of working with recessive genes is particularly evident with hybrid crops such as sorghum or maize. For all hybrid progeny to express the trait, both parents must be homozygous for the recessive gene. This can require many crosses and breeding cycles, in order to ensure homozygosity for the alleles. In contrast, a dominant trait gene that is homozygous in one parent is sufficient to ensure that all progeny plants express this trait in the hybrid progeny, regardless of the 2^{nd} parent's genetic makeup at that locus.

Commercial plant breeders are looking for many specific traits in each plant. Hence, dominant gene traits are highly desired due to the ability to more rapidly and accurately select desired lines. These traits of interest are quickly identified and those plants without the desired trait can be eliminated. These direct visual assays are immediate, saving time and expense of sample collection, DNA extraction and molecular marker analysis to identify the probable presence of a recessive gene. These time savings are compounded with each additional trait that is dominant rather than recessive.

The ability to convert a recessive gene to a dominant one would greatly improve the efficiency of commercial breeding programs.

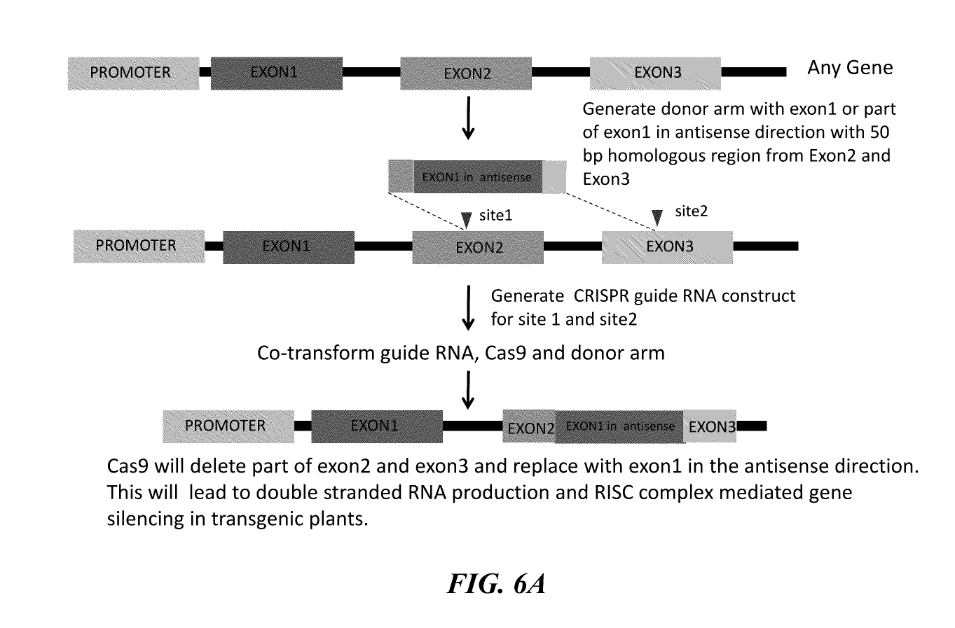

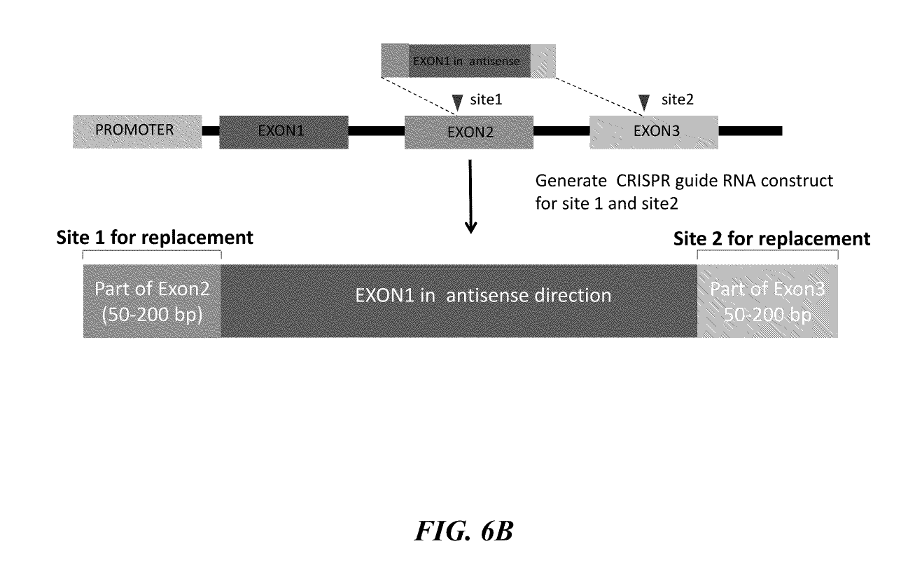

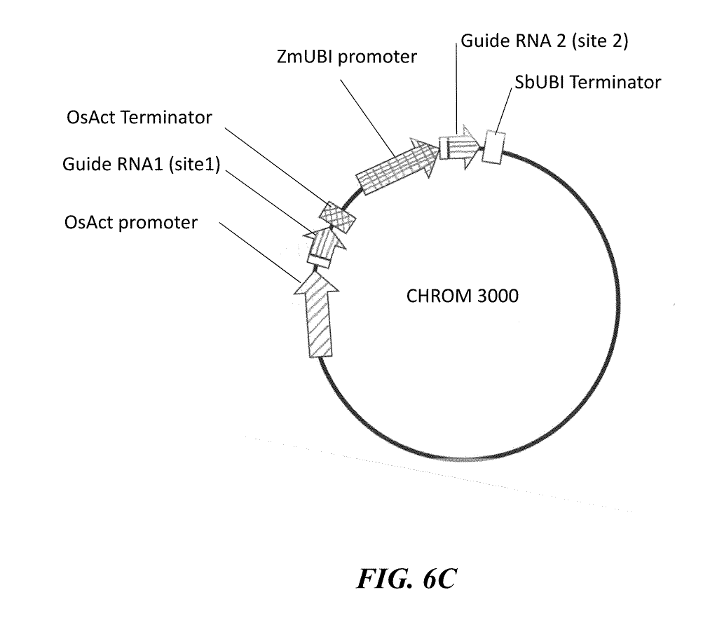

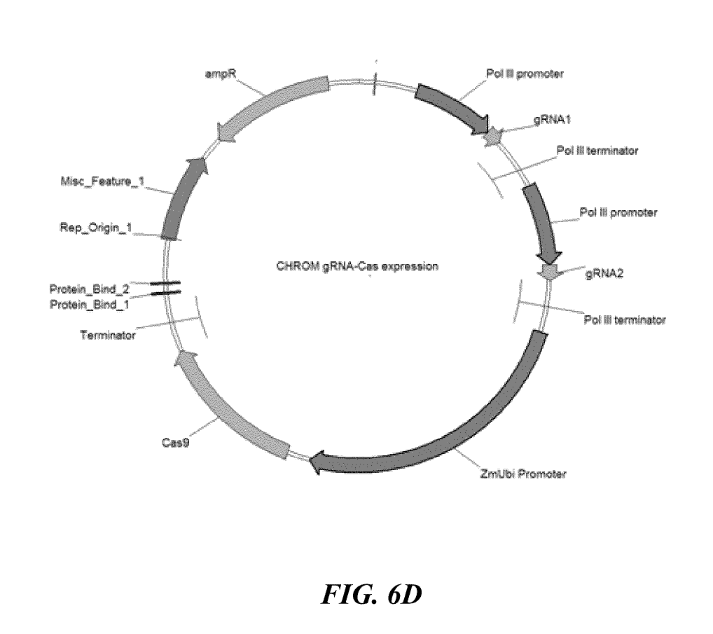

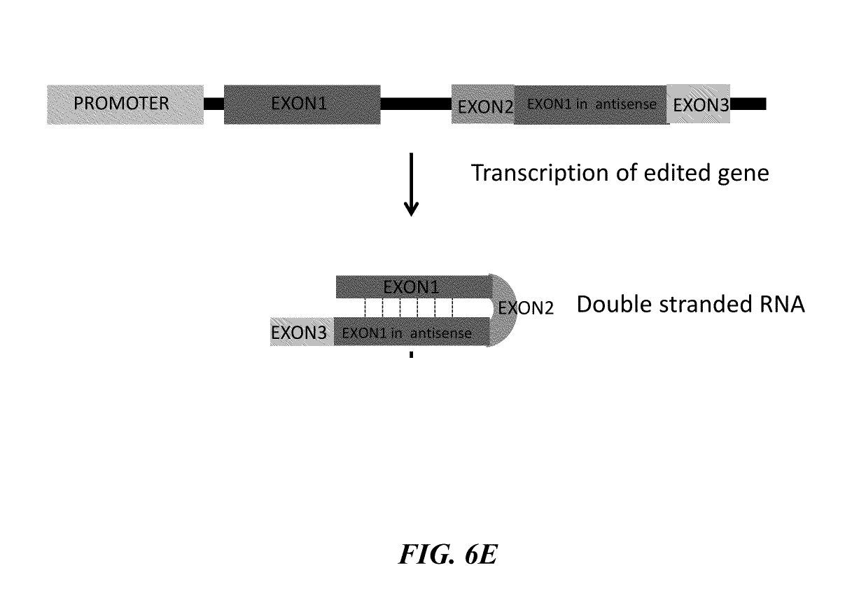

FIG. 6A shows a diagram schematically illustrating a method for CRISPR-Cas-mediated gene replacement in accordance with one embodiment of the present technology. In the example illustrated in FIG. 6A, a target gene is identified and a donor arm is generated. Referring to the illustrated example of FIG. 6A, the donor arm is designed to replace a portion of the target gene with an antisense sequence of a remaining exon. For example the schematic target gene in FIG. 6A shows replacement of a portion of exon 2 and exon3 with an antisense segment of exon 1. In the design of the donor arm (shown with additional detail in FIG. 6B), an antisense of exon 1 or a portion of exon 1 can be flanked with a short sequence (e.g., about 50 bp) of homologous region from exon 2 and exon 3, respectively. It will be understood that in embodiments having a greater number of exons, the donor arm can be suitable to replace larger portions of genomic sequence such that a resultant dsRNA transcript can have between about 50 bp to about 2000 bp. As shown in FIGS. 1A and 1E, an intervening exon portion (shown as the 5′ portion of exon 2) remains to form the hairpin turn. In these arrangements, the replaced exons are 3′ of the exon sequence that is targeted for forming a double stranded RNA. For example, the resulting edited/replaced gene will produce double stranded RNA (FIG. 1E) that will be recruited by the RISC complex for RNA degradation and production of 20- to 25-bp RNA fragments. RNA degradation will lead to post transcription gene silencing since the RNA transcript level available for translation into functional protein is reduced to none or to levels that contribute to plant phenotypes.

Once the donor arm is generated, cells can be co-transformed with the donor arm and plasmids carrying CRISPR guide nucleotide sequences for generating guide RNA (FIG. 1C) and a plasmid for generating Cas9 endonuclease, and using techniques known in the art. In certain embodiments, a suitable CRISPR-Cas9 construct can include CRISPR guide nucleotide sequences for generating guide RNA and include nucleotide sequence for generating a Cas9 endonuclease transcript (FIG. 6D). Suitable methods include any method by which DNA can be introduced into a cell, such as by Agrobacterium or viral infection, direct delivery of DNA such as, for example, by PEG-mediated transformation of protoplasts, by desiccation/inhibition-mediated DNA uptake, by electroporation, by agitation with silicon carbide fibers, by acceleration of DNA coated particles, etc. The transformed material can be introduced into any plant, algal or animal cell. In certain examples, the material can be transformed into protoplasts, embryos, tissue, portions of plants, algae cells, etc.

Referring back to FIGS. 6A-6E, and following transformation of the CRISPR-Cas vector(s) and donor arm, Cas9 endonuclease will replace part of exon2 and exon3 with the portion of exon1 in the antisense direction (from the donor arm). Transcription of the modified gene from its own endogenous promotor will yield a double stranded RNA transcript and RISC complex-mediated gene silencing in the targeted cell(s) (FIG. 6E).

While CRISPR-Cas-mediated gene modification is illustrated in this example, it will be understood that other gene editing/gene replacement methodologies (e.g., TALENs, Zinc Fingers, etc.) may be employed to induce modification of endogenous loci with a donor arm as discussed herein.

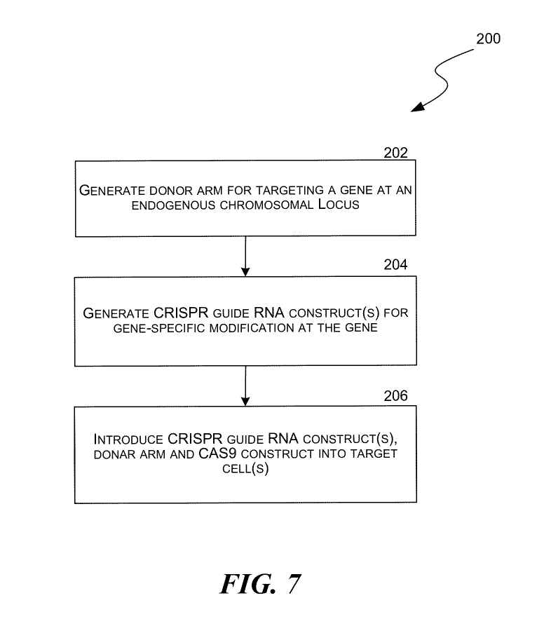

FIG. 7 shows a flow diagram illustrating a method 200 for editing a gene in accordance with an aspect of the present technology. Following selection of a target gene in a selected species (e.g., a plant species, animal, algal), and in one embodiment, the method 200 includes generating a donor arm for targeting a gene at an endogenous chromosomal locus (block 202). The donor arm can include an antisense sequence of a targeted exon in the gene flanked by two different targeted exon regions located 3′ of the targeted exon that can be used for homologous recombination to replace at least portions of the remaining exons with the antisense sequence. The method 200 can also include generating CRISPR guide RNA construct(s) (e.g., vectors) and CAS9 construct for targeted gene-specific modification at the gene (block 204). The method 200 can further include introducing CRISPR guide RNA construct(s), the donor arm and CAS9 construct into the target cell(s) (block 206). The method can induce gene modification at the endogenous chromosomal locus such that transcription of the edited gene (e.g., under its endogenous promotor) will produce a double-stranded RNA (shown in FIG. 1E). The double stranded-RNA will be to siRNA by the RISC complex which can lead to inhibition of gene expression. In a particular example of modifying a plant gene, down-regulation of a targeted gene is driven by the endogenous plant promotor which promotes a dominant trait that can be detected in the T0 plant generation.

### Example 1

### Targeted Mutagenesis of SbCAD2 Using CRISPR-Cas9 to Generate a Dominate Phenotype

One of the sorghum brown midrib (bmr) mutants (Porter et al. 1978), bmr6, is similar to the maize brown midrib1 (bm1) mutant, which has decreased CAD activity and contains cell walls with higher levels of cinnamaldehydes (Sallabos et al. 2008). The sorghum CAD2 (SbCAD2) is the predominantly expressed CAD gene in sorghum indicated that it is highly likely to be the main sorghum CAD involved in cell wall lignifications. In addition, a mutation in this gene is linked to the bmr phenoype (Sallabos et al. 2009).

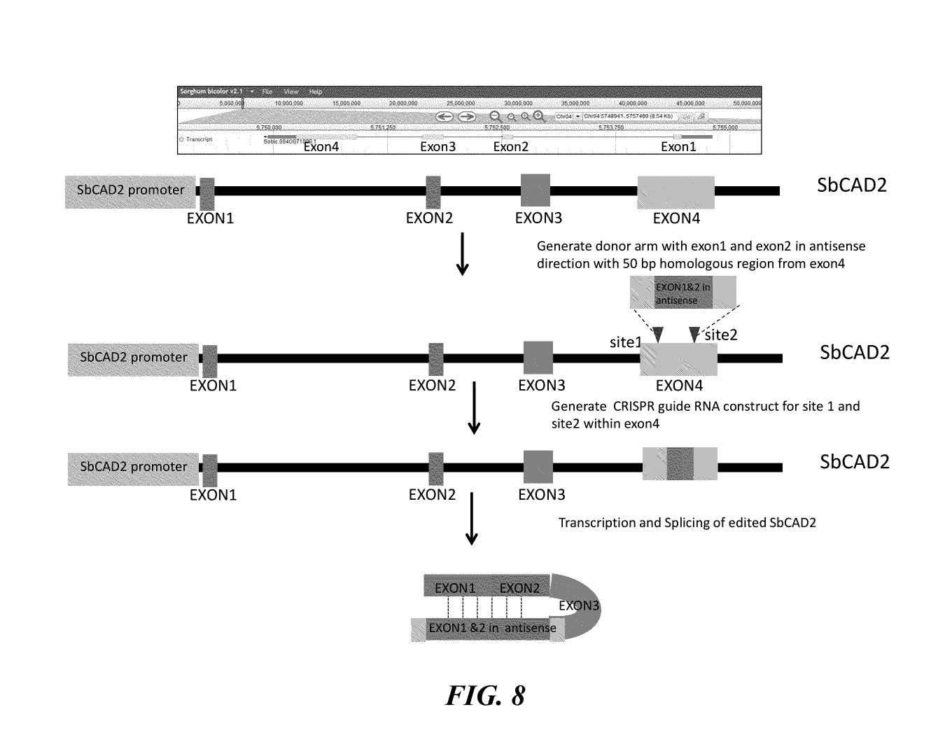

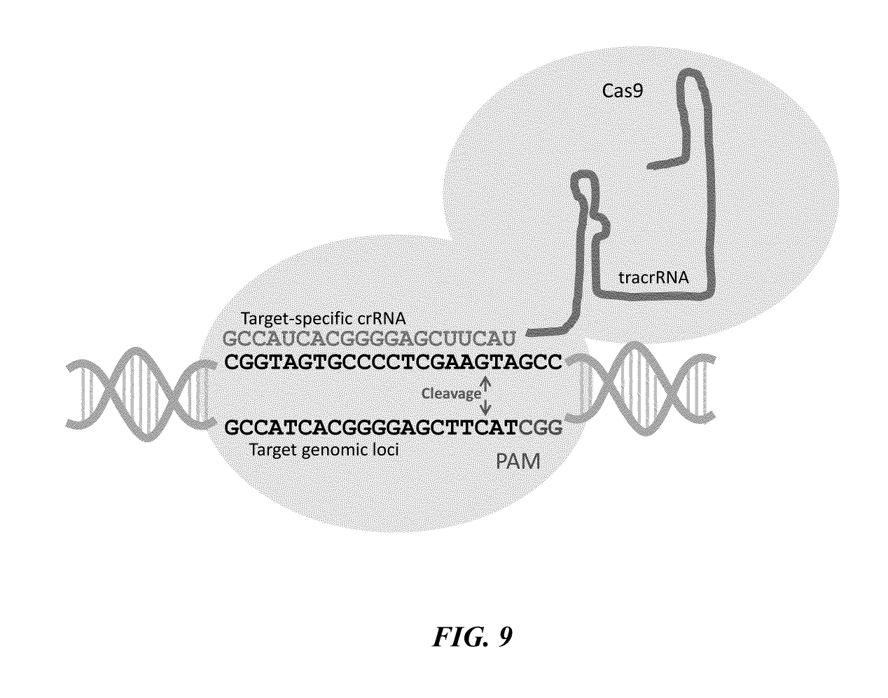

The CRISPR-Cas9-mediated methodology described above for generating a dominant phenotype in sorghum having reduced cell wall lignification is presented in this example. FIG. 8 shows a diagram schematically illustrating method steps for targeting the Sorghum bicolor CAD2 gene in a manner that generates SbCAD2 double-stranded RNA of in accordance with an embodiment of the present technology. In this example, a donor arm with exon1 and exon2 in an antisense direction flanked by two spaced apart 50 bp homologous regions from an internal portion of exon 4 is generated. A CRISPR guide sequence construct is generated for targeting site 1 and site 2 within exon 4 of sbCAD2. While a single vector can be used to produce both guide RNA constructs (e.g., targeting site 1 and site 2, respectively), one of ordinary skill in the art will understand that separate vectors carrying each guide sequence could be generated and co-transformed. Additionally, the Cas9 transcript can be generated from the same or a different vector construct. The donor arm, CRISPR guide sequence construct(s) and Cas9 vector construct (if different) is used to transform sorghum (e.g., cells, protoplasts, embryos, plant tissue, etc.). CRISPR-mediated gene modification is facilitated by the targeting of the homologous regions of the donor arm and the guide RNA (shown in FIG. 9). Referring back to FIG. 8, the modified sbCAD2 is transcribed from its endogenous promotor and forms a double-stranded RNA.

The sequences of SbCAD2 (genbank ID: AB288109.1; Sb04g005950) are shown here:

| Protein sequence of SbCAD2 | |
|---|---|
| (SEQ. ID. No. 64) | |
| 1 | mgslaserkv vgwaardatg hlspytytlr ntgpedvvvk vlycgichtd ihqaknhlga |
| 61 | skypmvpghe vvgevvevgp evskygvgdv vgvgvivgcc recspckanv eqycnkkiws |
| 121 | yndvytdgrp tqggfastmv vdqkfvvkip aglapeqaap llcagvtvys plkafgltap |
| 181 | glrggivglg gvghmgvkva kamghhvtvi sssskkraea mdhlgadayl vstdaaamaa |
| 241 | aadsldyiid tvpvhhplep ylsllrldgk hvllgvigep lsfvspmvml grkaitgsfi |
| 301 | gsidetaevl qfcvdkglts qievvkmgyv nealerlern dvryrfvvdv agsnveedaa |
| 361 | dapsn* |
| Complete coding DNA sequence of sbCAD2 | |
| (SEQ. ID. No. 65) | |
| 1 | gatcgcccac cctctcggcc tctccaggcc gccgccggct ccgtcgtcgt gttccccgac |
| 61 | gcccgtagcg ttcgaccgcg gccagtccca gtccaagagg agaatgggga gcctggcgtc |
| 121 | cgagaggaag gtggtcggct gggccgccag ggacgccacc ggacacctct ccccctacac |
| 181 | ctacaccctc aggaacacag gccctgaaga tgtggtggtg aaggtgctct actgtggaat |
| 241 | ctgccacacg gacatccacc aggccaagaa ccacctcggg gcttcaaagt accctatggt |
| 301 | ccctgggcac gaggtggtcg gtgaggtggt ggaggtcggg cccgaggtga gcaagtatgg |
| 361 | cgtcggcgac gtggtaggcg tcggggtgat cgtcgggtgc tgccgcgagt gcagcccctg |
| 421 | caaggccaac gttgagcagt actgcaacaa gaagatctgg tcctacaacg atgtctacac |
| 481 | tgacggccgg cccacgcagg gcggcttcgc ctccaccatg gtcgtcgacc agaagtttgt |
| 541 | ggtgaagatc ccggcgggtc tggcgccgga gcaagcggcg ccgctgctgt gcgcgggcgt |
| 601 | gacggtgtac agcccgctaa aggcctttgg gctgacggcc ccgggcctcc gcggtggcat |
| 661 | cgtgggcctg ggcggcgtgg gccacatggg cgtgaaggtg gcgaaggcca tgggccacca |
| 721 | cgtgacggtg atcagctcgt cgtccaagaa gcgcgcggag gcgatggacc acctgggcgc |
| 781 | ggacgcgtac ctggtgagca cggacgcggc ggccatggcg gcggccgccg actcgctgga |
| 841 | ctacatcatc gacacggtgc ccgtgcacca cccgctggag ccctacctgt cgctgctgag |
| 901 | gctggacggc aagcacgtgc tgctgggcgt catcggcgag cccctcagct tcgtgtcccc |
| 961 | gatggtgatg ctggggcgga aggccatcac ggggagcttc atcggcagca tcgacgagac |
| 1021 | cgccgaggtg ctccagttct gcgtcgacaa ggggctcacc tcccagatcg aggtggtcaa |
| 1081 | gatggggtac gtgaacgagg cgctggagcg gctcgagcgc aacgacgtcc gctaccgctt |
| 1141 | cgtcgtcgac gtcgccggca gcaacgtcga ggaggatgcc gctgatgcgc cgagcaactg |
| 1201 | acggcgtgca acgttcgttc ggggctcgag gctgcctgcg cttctgcttc ctttagtaat |
| 1261 | tgtgggcttt gtgcgttctt gccgtgttct gttctggttc tgggctttca gatgagttga |
| 1321 | aggatggtct gtttaaatgg catcagactg aataactata tgttgtagta gtacgtgtta |
| 1381 | tactcggagt acgccacgat atggtgtggt gtcagtgtca ccagcattct ggatttgcag |
| 1441 | tttacccaaa aaaaaaa |
| Genomic DNA of SbCAD | |
| (SEQ. ID. No. 66) | |
| 1 | gttgttggac catttataat ttttctccag tagccaccgc agaagatcct gctggcaggt |
| 61 | ggcctgccgg ttgccggact gccacttttg cacagcgccg atcgagctcg gctctccgac |
| 121 | tgcccctata tagcgcgcac tccgctcacg catttttttc ctaccaaaaa gacaggcgca |
| 181 | ctagttgtcg cgcggctttc tttcccgaag gctgagccgg gctcgtccgt ctccatcgcc |
| 241 | caccctctcg gcctctccag gccgccgccg gctccgtcgt cgtgttcccc gacgcccgta |
| 301 | gcgttcgacc gcggccagtc ccagtccaag aggagaatgg ggagcctggc gtccgagagg |
| 361 | aaggtggtcg gctgggccgc cagggacgcc accggacacc tctcccccta cacctacacc |
| 421 | ctcaggtacg ccgctccgcc gccgccgccg ccactctaga tcgctcgtgt tcgtcttctc |
| 481 | acttttccta cccctagtcc cctccccctt catgtccgtc cgactgtgtc tcctgctcct |
| 541 | tgtgcaaaca cgaaaataga tccaggagag gatgagggac ggtttggctt gtgcggcgcc |
| 601 | ttcttcagtg attgtccgag atcgaccagg aacaggaaga acagtaaaat ctgagtcatg |
| 661 | attgtgatga tttttttttt aaaaaaaaaa acaggatata tttccgatcc acttccacga |
| 721 | ttaggccggt gcacgtatct aatcgccggc aggttttaat ttgggaagga tgctatacgt |
| 781 | atgcatattc tgatccatat actataactg atacgtttac ggttatcatt taccgagtat |
| 841 | tccttctctt gatttctgta agatgttcct tatgttatat gctgtggtcg tatctttttc |
| 901 | ctcacacata ctgtagtata ctagtacacc ttagtaggag cactactcca caacaaacgc |
| 961 | atgcatgcgc atgcgcgcgg cagcatgcgc atgataggtc ttcaactcca ggtccaactc |
| 1021 | tagtgccgcc gcacatgcat gtatggatgc cacggttgag gatatatttt gcttcaatat |
| 1081 | taatatttgt gccctgcacc tgcactgcac gtgagtttga cgacgtttcg tacagaccca |
| 1141 | gtagccaacg tgttgtgtgg agtagcttgt cgtactggca ggtacaatac cagcaaacct |
| 1201 | aaaatatgga tacgggtgat gacaccgtac ctacagctac ctaccacctg gtagctgttt |
| 1261 | gcaacactgg cctggcgcgc gcacaccata attcttaaat tttttttgtt tggttattgt |
| 1321 | agcattttgt ttgtatttga taattattgt taatcatgga ttaactaagc tcaaagaatt |
| 1381 | catctagcaa atgacagtta aactgtacca ttagttatta tttttgttta tatttaatac |
| 1441 | ttcattatgt ggcgtaagat tcgatgtgat gaagaatctt aaaaagtttt ttggattttg |
| 1501 | gggtaaacta aacaagaact agttggcgaa aaaatttggg tttggctatt atagcacttt |
| 1561 | tgtttaattt gtatttgaca attattatcc cattaaagac tagctaggct caaaagattc |
| 1621 | gtctcgcaaa ttaaatgcaa cctgtgcaat tagttatttt ttaatctata tttaatgctc |
| 1681 | catgtatgtg tccaaagatt tgatatgacg gaaaattttg aaaaaataga aaatttttgg |
| 1741 | aactaaacag cctttataag tgatattatt ccgatcaggc tggaggaaat tgaacagcca |
| 1801 | tgggtttgtt tactcatata taagtgatcg atactgttga ttattccgat caggctggag |
| 1861 | gaaattgaac agcactacat aaacccttgg ctttcggttc attaagtagt agtagtctta |
| 1921 | atagtagtag tggtcactag gttatgtggt gcagtaattt gaaagcatcc atccatcgcc |
| 1981 | tgcatatact tttattattg cttcgagaga agactcttgc actgctttct catgtcatca |
| 2041 | actactagtg tacgatgata ctatctagct aactgtggcg gttcttgcat atttctatat |
| 2101 | gctgctggtc cttctgcaag aataaactaa ttaacactgg tctcttttta tatgggatgt |
| 2161 | gctgtgggtg acaacaacaa aaacaggaac acaggccctg aagatgtggt ggtgaaggtg |
| 2221 | ctctactgtg gaatctgcca cacggacatc caccaggcca agaaccacct cggggcttca |
| 2281 | aagtacccta tggtccctgg gtgagcacaa acaaaccccc tagctagcga ttttattttt |
| 2341 | cagcaccttt gggatcgagt aatactctgt atatggttta cgataaactg aattttccag |
| 2401 | tgttctatta ttcaaactgt ctgaaaagta taaatgaata ggacacatat atagcgacat |
| 2461 | gccgtttccg cattttgatg agaaaactac acatgcagac aaatttaggt atatctatct |
| 2521 | gattgacctg catagactgg tagataggtc agtgcacatt tggtaactac aaacgtcagc |
| 2581 | atctcagtcc gtagctattc ttagatttac aggtggcaca taccacacta aaactctttg |
| 2641 | ttacgtagtt ggttgccaat tactgtcatt ccatcagttt accaaattat ttgaagcaca |
| 2701 | agagtttgtt gcgtctaaga tgttcttttc atgatagcta aagagctgca gaaatgagta |
| 2761 | gtaaagcaaa ccccaccggc cggcctatat accttttttc tgacatgttt gcgaggggga |
| 2821 | aaaaaattaa ataaacataa acttttcctg acagcacaac cactccacta ctgcgaactg |
| 2881 | ataatgtgca cactagctat catgggttgg tttttgctaa tgtcgtgtgt ctgaaacttt |
| 2941 | tgcaggcacg aggtggtcgg tgaggtggtg gaggtcgggc ccgaggtgag caagtacggc |
| 3001 | gtcggcgacg tggtaggcgt cggggtgatc gtcgggtgct gccgcgagtg cagcccctgc |
| 3061 | aaggccaacg ttgagcagta ctgcaacaag aagatctggt cctacaacga tgtctacact |
| 3121 | gacggccggc ccacgcaggg cggcttcgcc tccaccatgg tcgtcgacca gaagtgagtt |
| 3181 | tcttgaaact gaaaactaat catcaggttc attcagcgtt atcttgcctg cagtgttcta |
| 3241 | gctagagata atttcttgtt tttttttttt aaaaaaagtt ggtctgaagt ctgaactaag |
| 3301 | caagaaatag ttgagcttca gtttgaactt ttgtggaagt ggatggtgat gtccaatcct |
| 3361 | tctagaaaag gtggagggga gagtatatgg gtatgggaaa aaatttatca ttgagagagt |
| 3421 | ccatcatcgt ccagctgcaa gtcagcgtat ggatgccttg tggtgaccag gcaagagtgt |
| 3481 | gatgtgaaaa gtacgacgtg gtgtgcttta ctggctcatc tttgtcaagt tgaaccataa |
| 3541 | ccacagaagc cgaatcctca cctactactc actactcatg tctgaagatt ggtcatccaa |
| 3601 | accatcactg gttgttggga gaaatgggga taactttctc catcgtttga ttccaaactt |
| 3661 | gcctgcgact ttagtgtact gtctttttca gtcagtgggc aaatcacact acctaatcca |
| 3721 | acaactcttt gagatagcga ttgcttgttt ttttttaaaa aaaaaatggg atatatgtgt |
| 3781 | gaattatgat agaacagtaa ctcctgaagc tattttattt ggtgctagtt aaatactatc |
| 3841 | caacaactct ttgagatagc gattgcttgt tgataattaa tgcattttgt ttcaggtttg |
| 3901 | tggtgaagat cccggcgggt ctggcgccgg agcaagcggc gccgctgctg tgcgcgggcg |
| 3961 | taacggtgta cagcccgcta aaggcctttg ggctgacggc cccgggcctc cgcggtggca |
| 4021 | tcgtgggcct gggcggcgtg ggccacatgg gcgtgaaggt ggcgaaggcc atgggccacc |
| 4081 | acgtgacggt gatcagctcg tcgtccaaga agcgcgcgga ggcgatggac cacctgggcg |
| 4141 | cggacgcgta cctggtgagc acggacgcgg cggccatggc ggcggccgcc gactcgctgg |
| 4201 | actacatcat cgacacggtg cccgtgcacc acccgctgga gccctacctg tcgctgctga |
| 4261 | ggctggacgg caagcacgtg ctgctgggcg tcatcggcga gcccctcagc ttcgtgtccc |
| 4321 | cgatggtgat gctggggcgg aaggccatca cggggagctt catcggcagc atcgacgaga |
| 4381 | ccgccgaggt gctccagttc tgcgtcgaca aggggctcac ctcccagatc gaggtggtca |
| 4441 | agatggggta cgtgaacgag gcgctggagc ggctcgagcg caacgacgtc cgctaccgct |
| 4501 | tcgtcgtcga cgtcgccggc agcaacgtcg aggaggatgc cgctgatgcg ccgagcaact |
| 4561 | gacggcgtgc aacgttcgtt cggggctcga ggctgcctgc gcttctgctt cctttagtaa |
| 4621 | ttgtgggctt tgtgcgttct tgccgtgttc tgttctggtt ctgggctttc agatgagttg |
| 4681 | aaggatggtc tgtttaaatg gcatcagact gaataactat atgttgtagt agtacgtgtt |
| 4741 | atactcggag tacgccacga tatggtgtgg tgtcagtgtc accagcattc tggatttgca |
| 4801 | gtttacccaa atgtttctgg tgctgcgtct cctacactgg gctaaccttt ttcagacgta |
| 4861 | tgcccaaatg |
| Putative promoter sequence of SbCAD2 (up to about 3 kb from ATG) | |
| (SEQ. ID. No. 67) | |
| 1 | ttaattgacg tatttggtct ttttgttcat tacaatgttg aatgttcaat acaaaaagtt |
| 61 | ctcgttgcta attaattaga aaacagcacg ttattaatta tataaaagaa ataaaacaaa |
| 121 | taaaactgca ggaaccgtag acttcgtgca tgaaaagatt aatgctagca tagaaaaaga |
| 181 | ctataactac cctaatctag ctagagtcaa tatgtatgaa acactctgga ttagggtgcc |
| 241 | ttaaccaact tatatatgct tcgaagtgag tctgaattcc ggatagctaa ttagttatta |
| 301 | gaattatagg tcagtcttag taaaagtttc attagggttt catttgcatt gtcacataag |
| 361 | cgcgcacttt tgatgatgtg acaacgtttt taaaaagaga gggaagatgt aagttttaca |
| 421 | gggatgaaac tcttttagta cgattatcaa cactttatta gtcatgaaat gaaagatcta |
| 481 | tatctacaaa accatagaat aaattttttc attgagatga tgtttcttac atgttttatt |
| 541 | ttattctata tgacatgata ttcttgaaaa ataacgttac aaaactctct attaagactt |
| 601 | accttagtta ttgtttgaat cctccagcta gctagtagtt aattgcattt agatagagat |
| 661 | agagagagcc agctatttag ctgagatatt tggatggaag cagccaacag taattagctg |
| 721 | tgcagtggag tattttagct agctgaaggg aggctttaat ttgggtttgt tcgaaaggtg |
| 781 | acgtggtcct gacgtcagat cctgcgggcc ccactcacct accacgccca acgacccccg |
| 841 | gcatcccttt cacgtttgtc atcctcctcg cggcttatca atatcaactg cctcttcgcg |
| 901 | gcacgtcact tttctcccat gcatcagcca gctcctcgtg cgcccaatct ctacttcatt |
| 961 | tgctcctgat ttgctcccat gcagaatcta cggacaaatc aacccaccac tggaaattaa |
| 1021 | aacgtacgat tctgattgcc gaagaaacaa gcacctattg cttctccctc cgtagcatgg |
| 1081 | aaagagtatt cgatattttt ttcttttaga acatagagtt ttgtaactct taaaagagta |
| 1141 | ttgagaggaa taaagaatgt cagctttaag acttttcaat aatccgctct taaaatatag |
| 1201 | aaacaatttt acatcatgat tcatatacta attctatctc ttctctcttg tatatttaat |
| 1261 | ttacctcagt aactttttcc tactctttgt tttcttcacg cccttccacc tttagattag |
| 1321 | ccgacccatg cacatcaaaa agaaaatacg catgacttga agtctgcgga actttacacg |
| 1381 | caaataggag ttttttctcc caagtgccaa aagattggga gatggatttt ttttttcatt |
| 1441 | ctttccaaga atcatgaatt gaaaaagatt attgggtact tttggagatg ctcattctct |
| 1501 | tgtttataaa taatatagta gttaatgtta ttctaaacgg taaatggatt aacgtttaaa |
| 1561 | cactcttgaa atggtttaaa taaaatgttt atatggtatt accaaaatat gcatatctgt |
| 1621 | tcatgtaaaa aagcttaata tgctaacaaa gatatataag tgtatattct aagaaaatta |
| 1681 | gttggctgcc aacaatatgg tggataggat cctcataccg gttaaattat taattaaatg |
| 1741 | tctatttatc atgtctaatc atgtttatcc ctttcgcgtt gtcttcctcc tcacggctta |
| 1801 | tcgatatcaa ctcagcttct tcgtggcaca tcactttaat ttcctcccat cagtgggttc |
| 1861 | ctccatctct acttcattag ctcccatgca gaattatact aataacaaat caatccaccc |
| 1921 | gccgctggaa aatgtgcgaa gaaacagcac ctactgctcc ctccctagca cggaatggat |
| 1981 | gatgtcaact ctctcttgtc tataatagta gttatcagtc ttattctaaa cggtaaaggg |
| 2041 | attaacgtgt agacaccgtt taaatggcct aaacaattct ttatatatta ccaaaatatg |
| 2101 | catgtctatt catgtaaaaa agtttaatat ggtaaaaaag atatataaat atgtatgtaa |
| 2161 | aagtgcatat tctaagaaaa aaaatatgta tgtagactca aatatttttt tttacatttc |
| 2221 | ctttctttta tttagtgcgg aacgaatagt ttcagtcttg cagacatgtt tgaattcaat |
| 2281 | aatttcttga aagaacatca ctgatgaaac ccatataagc agcaggcaca ctctccttgt |
| 2341 | tatcaaactt attccaatga aattacgaat caccaatagc ttagtagcag cagccatgct |
| 2401 | taacatgaag attctacaat ggcaactgat acgcccaggt ctgcaatatt aaagatttag |
| 2461 | tttggttttc cttaactcat gtcaagtagc actattaaat cttcaggatt atgtacatcg |
| 2521 | ttcccatcaa attatctaag aaaatgatgt cacggtccat cgtatatact atggaatacc |
| 2581 | tttaaatatt tcatgaaact tgatttcatc ttattagaaa tagtttttat tttgttttct |
| 2641 | ttctttcctc tatatagtgg tgagcaatgc aaatccgccg caacacgcga gagagtattc |
| 2701 | atctatttct acagactatt aacatcatgt ttagaacatg agatttttcc ttttattttc |
| 2761 | tttccctacc ttattcctgt gaaattaaac gaaaattcta tgaaattcct ttgcaaaccc |
| 2821 | tacaaaaaat tcctacgtac attgcaaaca tgtagctcca aatgatttgt ccaatttgtc |
| 2881 | agtacataca gagcttggag ctgcggtgtt ttcttggctg acctacatat ggagccacgc |
| 2941 | tcatgctgac ctatcatccg gggggctgtg tacgatttgc cacttgccag tgggatcacg |

E-CRISP (Heigwer, F. et al. 2014), an online tool to design and evaluate CRISPR, to identify CRISPR guide sequences for targeting sbCAD2 gene was used. The E-CRISP identified genomic sequences within Exon 4 of sbCAD2 that can be used to generate guide sequences within Exon 4 are presented below. Any two of the identified genomic sequences listed below can be combined with a donor sequence for gene replacement in Exon4:

| | | | | | | SEQ. |
|---|---|---|---|---|---|---|
| | | | | | Nucleotide | ID. |
| Name | Length | Start | End | Strand | sequence | NO. |
| exon4_1 | 23 | 241 | 264 | plus | GGCGCGGACGCG | 68 |
| | | | | | TACCTGGT NAG | |
| exon4_2 | 23 | 66 | 89 | plus | AACGGTGTACAG | 69 |
| | | | | | CCCGCTAA NGG | |
| exon4_3 | 23 | 65 | 88 | plus | TAACGGTGTACA | 70 |
| | | | | | GCCCGCTA NAG | |
| exon4_4 | 23 | 89 | 112 | minus | GCCCGGGGCCGT | 71 |
| | | | | | CAGCCCAA NGG | |
| exon4_5 | 23 | 448 | 471 | plus | GCCATCACGGGG | 72 |
| | | | | | AGCTTCAT NGG | |
| exon4_6 | 23 | 336 | 359 | minus | GCGACAGGTAGG | 73 |
| | | | | | GCTCCAGC NGG | |
| exon4_7 | 23 | 337 | 360 | minus | AGCGACAGGTAG | 74 |
| | | | | | GGCTCCAG NGG | |
| exon4_8 | 23 | 12 | 35 | plus | GATCCCGGCGGG | 75 |
| | | | | | TCTGGCGC NGG | |
| exon4_9 | 23 | 97 | 120 | minus | CCGCGGAGGCCC | 76 |
| | | | | | GGGGCCGT NAG | |
| exon4_10 | 23 | 97 | 120 | minus | CCGCGGAGGCCC | 77 |
| | | | | | GGGGCCGT NAG | |
| exon4_11 | 23 | 238 | 261 | minus | ACCAGGTACGCG | 78 |
| | | | | | TCCGCGCC NAG | |
| exon4_12 | 23 | 238 | 261 | minus | ACCAGGTACGCG | 79 |
| | | | | | TCCGCGCC NAG | |

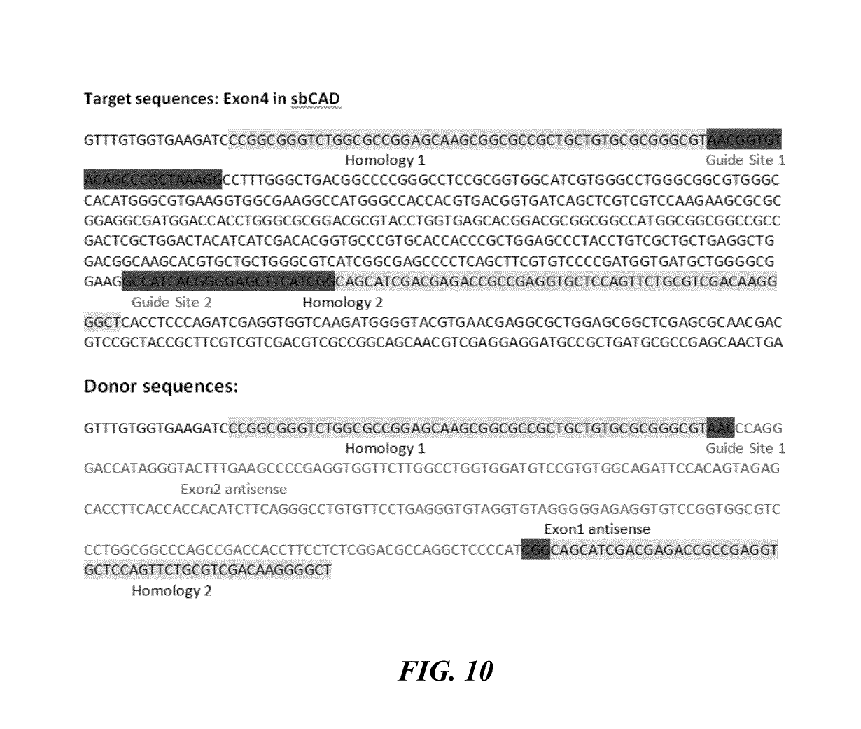

FIG. 10 illustrates target sequences and donor sequences for gene replacement in the SbCAD2 gene in accordance with one embodiment of the present technology.

| Target sequence for gene replacement of SbCAD2 | |
|---|---|
| (SEQ. ID. No. 80) | |
| 1 | gtttgtggtg aagatcccgg cgggtctggc gccggagcaa gcggcgccgc tgctgtgcgc |
| 61 | gggcgtaacg gtgtacagcc cgctaaaggc ctttgggctg acggccccgg gcctccgcgg |
| 121 | tggcatcgtg ggcctgggcg gcgtgggcca catgggcgtg aaggtggcga aggccatggg |
| 181 | ccaccacgtg acggtgatca gctcgtcgtc caagaagcgc gcggaggcga tggaccacct |
| 241 | gggcgcggac gcgtacctgg tgagcacgga cgcggcggcc atggcggcgg ccgccgactc |
| 301 | gctggactac atcatcgaca cggtgcccgt gcaccacccg ctggagccct acctgtcgct |
| 361 | gctgaggctg gacggcaagc acgtgctgct gggcgtcatc ggcgagcccc tcagcttcgt |
| 421 | gtccccgatg gtgatgctgg ggcggaaggc catcacgggg agcttcatcg gcagcatcga |
| 481 | cgagaccgcc gaggtgctcc agttctgcgt cgacaagggg ctcacctccc agatcgaggt |
| 541 | ggtcaagatg gggtacgtga acgaggcgct ggagcggctc gagcgcaacg acgtccgcta |
| 601 | ccgcttcgtc gtcgacgtcg ccggcagcaa cgtcgaggag gatgccgctg atgcgccgag |
| 661 | caactga |
| Guide sequence at site 1 | |
| (SEQ. ID. No. 81) | |
| 1 | gccatcacgg ggagcttcat cgg |
| Guide sequence at site 2 | |
| (SEQ. ID. No. 82) | |
| 1 | gccatcacgg ggagcttcat cgg |
| Donor Sequence | |
| (SEQ. ID. No. 83) | |
| 1 | gtttgtggtg aagatcccgg cgggtctggc gccggagcaa gcggcgccgc tgctgtgcgc |
| 61 | gggcgtaacc cagggaccat agggtacttt gaagccccga ggtggttctt ggcctggtgg |
| 121 | atgtccgtgt ggcagattcc acagtagagc accttcacca ccacatcttc agggcctgtg |
| 181 | ttcctgaggg tgtaggtgta gggggagagg tgtccggtgg cgtccctggc ggcccagccg |
| 241 | accaccttcc tctcggacgc caggctcccc atcggcagca tcgacgagac cgccgaggtg |
| 301 | ctccagttct gcgtcgacaa ggggct |

### Example 2

### Targeted Mutagenesis of SbCSE Using CRISPR-Cas9 to Generate a Dominate Phenotype

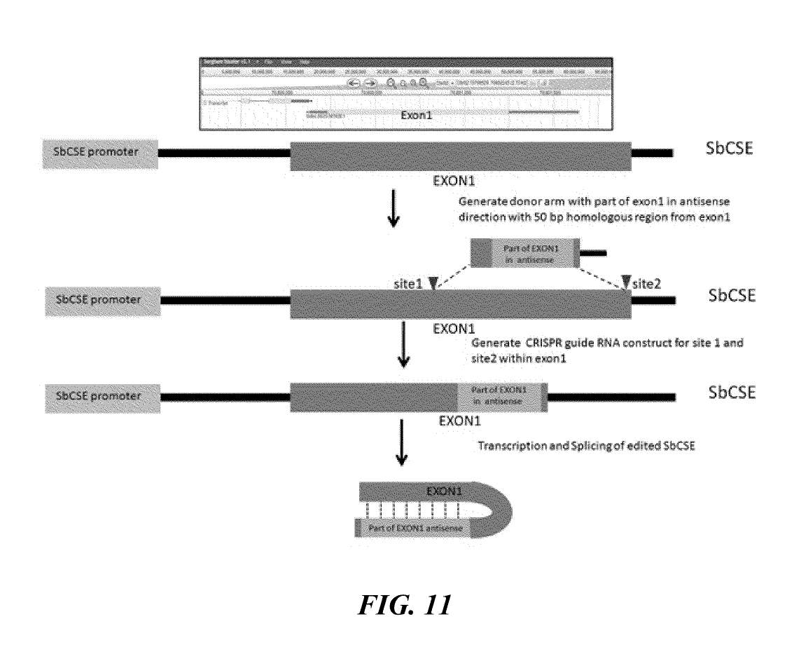

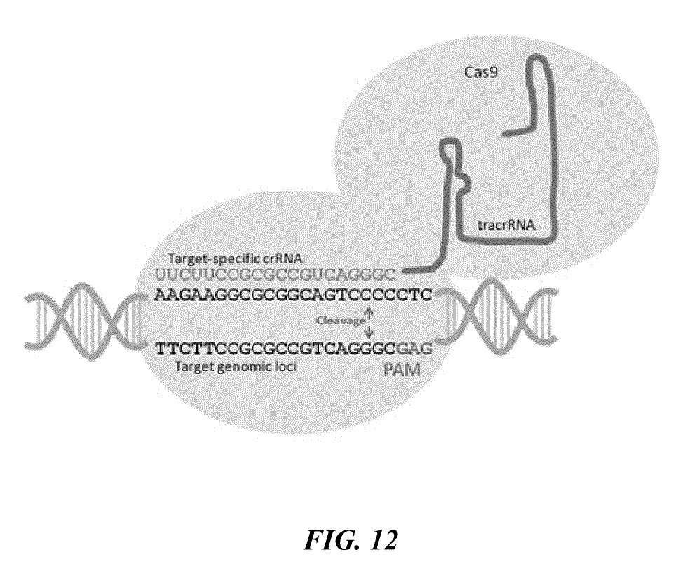

The CRISPR-Cas9-mediated methodology described above for generating a dominant phenotype in sorghum having reduced cell wall lignification is further presented in this second example. FIG. 11 shows a diagram schematically illustrating targeting and double-stranded RNA formation of the Sorghum bicolor CSE gene in accordance with an embodiment of the present technology. In this example, a donor arm with a portion of exon1 in an antisense direction and flanked by two spaced apart 50 bp homologous regions from a spaced-apart internal portion of exon 1 is generated. A CRISPR guide RNA construct is generated for targeting site 1 and site 2 within the spaced-apart portion of exon 1 of sbCSE. While a single vector can be used to produce both guide sequence constructs (e.g., targeting site 1 and site 2, respectively), one of ordinary skill in the art will understand that separate vectors carrying each guide sequence could be generated and co-transformed. The donor arm, CRISPR guide sequence construct(s) and CAS9 vector construct is used to transform sorghum (e.g., cells, protoplasts, embryos, plant tissue, etc.). CRISPR-mediated gene modification is facilitated by the targeting of the homologous regions of the donor arm and the guide RNA (shown in FIGS. 11 and 12). Referring back to FIG. 11, the modified sbCSE is transcribed from its endogenous promotor and forms a double-stranded RNA.

| The sequences of SbCSE are shown here: | |
|---|---|
| Genomic DNA of SbCSE | |
| (SEQ. ID. No. 84) | |
| 1 | ggtgatggag cgacatggtt cttaaaatca tttttttcat aaactaaaaa tcgaaaggtt |
| 61 | tattggccct aataatgtcg gtacacgagt taatgttccc tgcatgggcc aactatgaac |
| 121 | gagaatagta taccacgtgg acccgtgggc cgcggcacga gccgttccac ctacccgcaa |
| 181 | cgaaccgagc gatttcgccg tcccgcatcc aaacgccccc agcagccctt cccctgcccc |
| 241 | agtgccccgt cgcaactggc agcagcagcg accagcgact cccccaactc gccggccacc |
| 301 | agtagttccc tgcttcccca tcccatccac acacaccgca caccaaccaa ccccaccacg |
| 361 | ccaacgtccg ggaccaaact ctgatcccca ccatgcaggc ggacggggac gcgccggcgc |
| 421 | cggcgccggc cgtccacttc tggggcgagc acccggccac ggaggcggag ttctacgcgg |
| 481 | cgcacggcgc ggagggcgag ccctcctact tcaccacgcc cgacgcgggc gcccggcggc |
| 541 | tcttcacgcg cgcgtggagg ccccgcgcgc ccgagcggcc cagggcgctc gtcttcatgg |
| 601 | tccacggcta cggcaacgac gtcagctgga cgttccagtc cacggcggtc ttcctcgcgc |
| 661 | ggtccgggtt cgcctgcttc gcggccgacc tcccgggcca cggccgctcc cacggcctcc |
| 721 | gcgccttcgt gcccgacctc gacgccgccg tcgccgacct cctcgccttc ttccgcgccg |
| 781 | tcagggcgag ggaggagcac gcgggcctgc cctgcttcct cttcggggag tccatgggcg |
| 841 | gggccatctg cctgctcatc cacctccgca cgcggccgga ggagtgggcg ggggcggtcc |
| 901 | tcgtcgcgcc catgtgcagg atctccgacc ggatccgccc gccgtggccg ctgccggaga |
| 961 | tcctcacctt cgtcgcgcgc ttcgcgccca cggccgctat cgtgcccacc gccgacctca |
| 1021 | tcgagaagtc cgtcaaggtg cccgccaagc gcatcgttgc agcccgcaac cctgtgcgct |
| 1081 | acaacggtcg ccccaggctc ggcaccgtcg tcgagctgtt gcgtgccacc gacgagctgg |
| 1141 | gcaagcgtct cggcgaggtc agcatcccgt tccttgtcgt gcacggcagc gccgacgagg |
| 1201 | ttactgaccc ggaagtcagc cgcgccctgt acgccgccgc cgccagcaag gacaagacta |
| 1261 | tcaagatata cgacgggatg ctccactcct tgctatttgg ggaaccggac gagaacatcg |
| 1321 | agcgtgtccg cggcgacatc ctggcctggc tcaacgagag atgcacaccg ccggcaactc |
| 1381 | cctggcaccg tgacatacct gtcgaataag cattccaggc tgttcagatt ccgatgtatc |
| 1441 | gattacacaa gaaaattggt ttcatgtaca acgattctta tactatacgc tatatacttg |
| 1501 | gtcgtattt |
| Putative promoter sequence of SbCSE (up to about 2 kb from ATG) | |
| (SEQ. ID. No. 85) | |
| 1 | aaaattatgg ctaaaagtat tgtttactga tttattatgg aagaaaagca ctactgacta |
| 61 | gcagaaaaag tacggcttat aacacaaacg aacggaacct atgtactaac tattaactag |
| 121 | atcggtgcta aaatgtactc cctccattcc taaataaatt aaattctaga gttatcttaa |
| 181 | ataaaacttt tttaacgttt tactgaattt atagaaagaa acacaaatat ttatgacacc |
| 241 | aaatgatcat attataaaaa ttattatggt gtatctcatg atactaatat agtgtcataa |
| 301 | attttgacat ttttattaaa taaaataaaa tttagtcaaa ttttaaaaag ttggacttaa |
| 361 | ggcaaatcta aaagttgatt tattcaggaa tcagaggaag ttaaaaaaaa atgattccag |
| 421 | agctgttctt aaatttgttg caaacacatg gagggattgc ttaaagatac atgggctcag |
| 481 | gggatgctgc agtaccggta gcacctgccc tgagctggcg gacaactaaa atatttaagc |
| 541 | aaaaaaaatg atggctacga ttgtaaattg agcgtagttc agcaagtgaa cccaatccac |
| 601 | catgttcaaa tttttctatc ttttttctag aatttaacaa cgttgtgttt tttaatgtta |
| 661 | ggagacatgg tactatgatc aactgatcat ttcgttaacc tttttatgta cagcatcatc |
| 721 | gagcatgcac tggtccgaga tataggcagc ttaagcacca gttttatgtg cagccggata |
| 781 | ggtgatatgt ccttgctaat taggctccta tttgtagcta tagtattatc tattcatacg |
| 841 | gccctatcca ttgctaagag caagtataat aagttatttt tagccggttg caagagtcca |
| 901 | cctaatcaaa aaagcagacc acgtaggaga gatattaggg cactcacaat gcaagactct |
| 961 | atcacaaagt ccaagacaat taattacata ttatttatgg tattttgctg atgtggcagc |
| 1021 | atatttattg aagaaagagg tagaaaaaaa taagactcca agtcttattt agactctaag |
| 1081 | tccacattgt tcgaggtaat aaataacttt agactctatg atagagtctg cattgtgagt |
| 1141 | gcccttatag agccggcgat tcccatctcg cccgcctcta gctcaagata cgagaaaaaa |
| 1201 | aaatttgtcc tagacgtctt ccagcccgct gtgagcgcga tgccgacgct tccatctccc |
| 1261 | gccgttccgc tccctaattc tgtgctctac tcgatcatta cctgacatta aatacttgta |
| 1321 | tttttattat agtacacctc caagctggct aaaccatttt gatgtttagg ttagtacatg |
| 1381 | ttgatgttta ggttaggtgt aagtgatatg acaacttctc tcaaccgtca gccggctaaa |
| 1441 | ccattagcct tgctctaact gggctttatt tgttgctaca gtactagtat ctacaccttc |
| 1501 | ggtcgtaccc attttcacac tctatgaaaa cgctccgttt aatggaactt gttttctgct |
| 1561 | taatctgcca aggctctcgt tcatcaaaag aaaataaagc gagaatcagg tgatggagcg |
| 1621 | acatggttct taaaatcatt tttttcataa actaaaaatc gaaaggttta ttggccctaa |
| 1681 | taatgtcggt acacgagtta atgttccctg catgggccaa ctatgaacga gaatagtata |
| 1741 | ccacgtggac ccgtgggccg cggcacgagc cgttccacct acccgcaacg aaccgagcga |
| 1801 | tttcgccgtc ccgcatccaa acgcccccag cagcccttcc cctgccccag tgccccgtcg |
| 1861 | caactggcag cagcagcgac cagcgactcc cccaactcgc cggccaccag tagttccctg |
| 1921 | cttccccatc ccatccacac acaccgcaca ccaaccaacc ccaccacgcc aacgtccggg |
| 1981 | accaaactct gatccccacc |

Again using E-CRISP (Heigwer, F. et al. 2014), CRISPR guide sequences were identified for targeting the sbCSE gene. The E-CRISP identified genomic sequences within Exon1 of SbCSE that can be used to generate guide sequences within Exon 1 and these are presented below. Any two of the identified genomic sequences can be used with donor sequence for gene replacement in Exon1:

| | | | | | | SEQ. |
|---|---|---|---|---|---|---|
| | | | | | Nucleotide | ID. |
| Name | Length | Start | End | Strand | sequence | No. |
| exon1_1 | 23 | 126 | 149 | minus | AGCCGCCGGGCG | 86 |
| | | | | | CCCGCGTC NGG | |
| exon1_2 | 23 | 127 | 150 | minus | GAGCCGCCGGGC | 87 |
| | | | | | GCCCGCGT NGG | |
| exon1_3 | 23 | 142 | 165 | plus | GGCGGCTCTTCA | 88 |
| | | | | | CGCGCGCG NGG | |
| exon1_4 | 23 | 147 | 170 | minus | GGCCTCCACGCG | 89 |
| | | | | | CGCGTGAA NAG | |
| exon1_5 | 23 | 332 | 355 | minus | CGGCGTCGAGGT | 90 |
| | | | | | CGGGCACG NAG | |
| exon1_6 | 23 | 375 | 398 | plus | TTCTTCCGCGCC | 91 |
| | | | | | GTCAGGGC NAG | |
| exon1_7 | 23 | 504 | 527 | plus | GTCCTCGTCGCG | 92 |
| | | | | | CCCATGTG NAG | |
| exon1_8 | 23 | 700 | 723 | plus | CCAGGCTCGGCA | 93 |
| | | | | | CCGTCGTC NAG | |
| exon1_9 | 23 | 816 | 839 | minus | TACAGGGCGCGG | 94 |
| | | | | | CTGACTTC NGG | |
| exon1_10 | 23 | 989 | 1012 | minus | CGACAGGTATGT | 95 |
| | | | | | CACGGTGC NAG | |

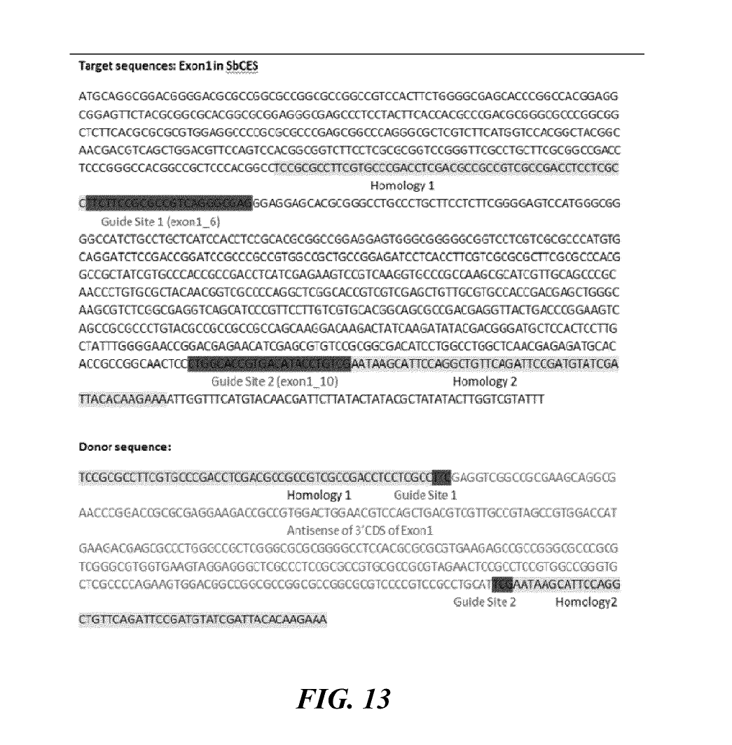

FIG. 13 illustrates target sequences and donor sequences for gene replacement in the SbCSE gene in accordance with one embodiment of the present technology.

| Target sequence of gene replacement of SbCSE | |
|---|---|
| (SEQ. ID. No. 96) | |
| 1 | atgcaggcgg acggggacgc gccggcgccg gcgccggccg tccacttctg gggcgagcac |
| 61 | ccggccacgg aggcggagtt ctacgcggcg cacggcgcgg agggcgagcc ctcctacttc |
| 121 | accacgcccg acgcgggcgc ccggcggctc ttcacgcgcg cgtggaggcc ccgcgcgccc |
| 181 | gagcggccca gggcgctcgt cttcatggtc cacggctacg gcaacgacgt cagctggacg |
| 241 | ttccagtcca cggcggtctt cctcgcgcgg tccgggttcg cctgcttcgc ggccgacctc |
| 301 | ccgggccacg gccgctccca cggcctccgc gccttcgtgc ccgacctcga cgccgccgtc |
| 361 | gccgacctcc tcgccttctt ccgcgccgtc agggcgaggg aggagcacgc gggcctgccc |
| 421 | tgcttcctct tcggggagtc catgggcggg gccatctgcc tgctcatcca cctccgcacg |
| 481 | cggccggagg agtgggcggg ggcggtcctc gtcgcgccca tgtgcaggat ctccgaccgg |
| 541 | atccgcccgc cgtggccgct gccggagatc ctcaccttcg tcgcgcgctt cgcgcccacg |
| 601 | gccgctatcg tgcccaccgc cgacctcatc gagaagtccg tcaaggtgcc cgccaagcgc |
| 661 | atcgttgcag cccgcaaccc tgtgcgctac aacggtcgcc ccaggctcgg caccgtcgtc |
| 721 | gagctgttgc gtgccaccga cgagctgggc aagcgtctcg gcgaggtcag catcccgttc |
| 781 | cttgtcgtgc acggcagcgc cgacgaggtt actgacccgg aagtcagccg cgccctgtac |
| 841 | gccgccgccg ccagcaagga caagactatc aagatatacg acgggatgct ccactccttg |
| 901 | ctatttgggg aaccggacga gaacatcgag cgtgtccgcg gcgacatcct ggcctggctc |
| 961 | aacgagagat gcacaccgcc ggcaactccc tggcaccgtg acatacctgt cgaataagca |
| 1021 | ttccaggctg ttcagattcc gatgtatcga ttacacaaga aaattggttt catgtacaac |
| 1081 | gattcttata ctatacgcta tatacttggt cgtattt |
| Guide sequence at site1 | |
| (SEQ. ID. No. 97) | |
| 1 | ttcttccgcg ccgtcagggc gag |
| Guide sequence at site 2 | |
| (SEQ. ID. No. 98) | |
| 1 | cgacaggtat gtcacggtgc cag |
| Donor sequence | |
| (SEQ. ID. No. 99) | |
| 1 | tccgcgcctt cgtgcccgac ctcgacgccg ccgtcgccga cctcctcgcc ttcgaggtcg |
| 61 | gccgcgaagc aggcgaaccc ggaccgcgcg aggaagaccg ccgtggactg gaacgtccag |
| 121 | ctgacgtcgt tgccgtagcc gtggaccatg aagacgagcg ccctgggccg ctcgggcgcg |
| 181 | cggggcctcc acgcgcgcgt gaagagccgc cgggcgcccg cgtcgggcgt ggtgaagtag |
| 241 | gagggctcgc cctccgcgcc gtgcgccgcg tagaactccg cctccgtggc cgggtgctcg |
| 301 | ccccagaagt ggacggccgg cgccggcgcc ggcgcgtccc cgtccgcctg cattcgaata |
| 361 | agcattccag gctgttcaga ttccgatgta tcgattacac aagaaa |

Further aspects of the present technology are directed to generation of sorghum breeding lines demonstrating desirable phenotypes through the conversion of recessive traits to dominate traits. As described herein, are methods for converting a recessive trait induced by mutations (such as Brown mid rib (bmr) mutation, multiseeded mutant (msd) or caffeoyl shikimate esterase mutation (cse)) to dominate traits without a transgenic (e.g., genetically modified organism) approach (e.g., conventional RNAi approach).

### REFERENCES

The following references are herein incorporated by reference in their entireties:
Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic acids research. 25:3389-3402.
Ausubel, F. M. 1987. Current protocols in molecular biology. Greene Publishing Associates; J. Wiley, order fulfillment, Brooklyn, N. Y. Media, Pa.
Beclin, C., S. Boutet, P. Waterhouse, and H. Vaucheret. 2002. A branched pathway for transgene-induced RNA silencing in plants. Current biology: CB. 12:684-688.
Bennett, W. F., B. Tucker, and A. B. Maunder. 1990. Modern grain sorghum production. Iowa State University Press, Ames. viii, 169 p. pp.
Bernstein, E., A. A. Caudy, S. M. Hammond, and G. J. Hannon. 2001. Role for a bidentate ribonuclease in the initiation step of RNA interference. Nature. 409:363-366.
Broach, J. R., J. N. Strathern, and J. B. Hicks. 1979. Transformation in yeast: development of a hybrid cloning vector and isolation of the CAN1 gene. Gene. 8:121-133.
Brummell, D., P. Balint-Kurti, M. Harpster, J. Palys, P. Oeller, and N. Gutterson. 2003a. Inverted repeat of a heterologous 3′-untranslated region for high-efficiency, high-throughput gene silencing. Plant J. 33:798-800.
Brummell, D., P. Balint-Kurti, M. Harpster, J. Palys, P. Oeller, and N. Gutterson. 2003b. Inverted repeat of a heterologous 3′-untranslated region for high-efficiency, high-throughput gene silencing. Plant J. 33:798-800.
Capecchi, M. R. 1980. High efficiency transformation by direct microinjection of DNA into cultured mammalian cells. Cell. 22:479-488.
Chuang, C. F., and E. M. Meyerowitz. 2000. Specific and heritable genetic interference by double-stranded RNA in Arabidopsis thaliana. Proceedings of the National Academy of Sciences of the United States of America. 97:4985-4990.
Clapp, D. W. 1993. Somatic gene therapy into hematopoietic cells. Current status and future implications. Clinics in perinatology. 20:155-168.
Curiel, D. T., S. Agarwal, E. Wagner, and M. Cotten. 1991. Adenovirus enhancement of transferrin-polylysine-mediated gene delivery. Proceedings of the National Academy of Sciences of the United States of America. 88:8850-8854.
Curiel, D. T., E. Wagner, M. Cotten, M. L. Birnstiel, S. Agarwal, C. M. Li, S. Loechel, and P. C. Hu. 1992. High-efficiency gene transfer mediated by adenovirus coupled to DNA-polylysine complexes. Human gene therapy. 3:147-154.
Curtain, S., D. F. voytas, and R. Stupar. 2012. Genome engineering of crops with designer nucleases. Plant Genome. 5:42-50.
Dafny-Yelin, M., and T. Tzfira. 2007. Delivery of multiple transgenes to plant cells. Plant physiology. 145:1118-1128.
Di Serio, F., H. Schob, A. Iglesias, C. Tarina, E. Bouldoires, and F. Meins, Jr. 2001. Sense- and antisense-mediated gene silencing in tobacco is inhibited by the same viral suppressors and is associated with accumulation of small RNAs. Proceedings of the National Academy of Sciences of the United States of America. 98:6506-6510.
Dien, B., G. Sarath, J. Pedersen, S. Sattler, H. Chen, and D. Funnell-Harris. 2009. Improved sugar conversion and thanol yield for forage sorghum (Sorghum bicolor L. Moench) lines with reduced lignin contents. Bioenergy Res. 2:153-164.
Djikeng, A., H. Shi, C. Tschudi, and E. Ullu. 2001. RNA interference in Trypanosoma brucei: cloning of small interfering RNAs provides evidence for retroposon-derived 24-26-nucleotide RNAs. Rna. 7:1522-1530.
Eamens, A. L., and P. M. Waterhouse. 2011. Vectors and methods for hairpin RNA and artificial microRNA-mediated gene silencing in plants. Methods in molecular biology. 701:179-197.
Eglitis, M. A., and W. F. Anderson. 1988. Retroviral vectors for introduction of genes into mammalian cells. BioTechniques. 6:608-614.
Eglitis, M. A., P. W. Kantoff, D. B. Kohn, E. Karson, R. C. Moen, C. D. Lothrop, Jr., R. M. Blaese, and W. F. Anderson. 1988. Retroviral-mediated gene transfer into hemopoietic cells. Advances in experimental medicine and biology. 241:19-27.
Fagard, M., and H. Vaucheret. 2000. (TRANS)GENE SILENCING IN PLANTS: How Many Mechanisms? Annual review of plant physiology and plant molecular biology. 51:167-194.
Fornale, S., M. Capellades, A. Encina, K. Wang, S. Irar, C. Lapierre, K. Ruel, J. P. Joseleau, J. Berenguer, P. Puigdomenech, J. Rigau, and D. Caparros-Ruiz. 2012. Altered lignin biosynthesis improves cellulosic bioethanol production in transgenic maize plants down-regulated for cinnamyl alcohol dehydrogenase. Molecular plant. 5:817-830.
Fromm, M., L. P. Taylor, and V. Walbot. 1985. Expression of genes transferred into monocot and dicot plant cells by electroporation. Proceedings of the National Academy of Sciences of the United States of America. 82:5824-5828.
Fu, C., J. R. Mielenz, X. Xiao, Y. Ge, C. Y. Hamilton, M. Rodriguez, Jr., F. Chen, M. Foston, A. Ragauskas, J. Bouton, R. A. Dixon, and Z. Y. Wang. 2011a. Genetic manipulation of lignin reduces recalcitrance and improves ethanol production from switchgrass. Proceedings of the National Academy of Sciences of the United States of America. 108:3803-3808.
Fu, C., X. Xiao, Y. Xi, Y. Ge, F. Chen, and J. Bouton. 2011b. Downregulation of cinnamyl alcohol dehydrogenase (CAD) leads to improved saccharification efficiency. Bioenergy Res. 4:153-164.
Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proceedings of the National Academy of Sciences of the United States of America. 90:11478-11482.
Gao, H., J. Smith, M. Yang, S. Jones, V. Djukanovic, M. G. Nicholson, A. West, D. Bidney, S. C. Falco, D. Jantz, and L. A. Lyznik. 2010. Heritable targeted mutagenesis in maize using a designed endonuclease. The Plant journal: for cell and molecular biology. 61:176-187.
Gendler, K., T. Paulsen, and C. Napoli. 2008. ChromDB: the chromatin database. Nucleic acids research. 36:D298-302.
Goodstein, D. M., S. Shu, R. Howson, R. Neupane, R. D. Hayes, J. Fazo, T. Mitros, W. Dirks, U. Hellsten, N. Putnam, and D. S. Rokhsar 2012. Phytozome: a comparative platform for green plant genomics. Nucleic acids research. 40:D1178-1186.
Graham, F. L., and A. J. van der Eb. 1973. Transformation of rat cells by DNA of human adenovirus 5. Virology. 54:536-539.
Grishok, A., A. E. Pasquinelli, D. Conte, N. Li, S. Parrish, I. Ha, D. L. Baillie, A. Fire, G. Ruvkun, and C. C. Mello. 2001. Genes and mechanisms related to RNA interference regulate expression of the small temporal RNAs that control C. elegans developmental timing. Cell. 106:23-34.
Guo, D., F. Chen, K. Inoue, J. W. Blount, and R. A. Dixon. 2001. Downregulation of caffeic acid 3-O-methyltransferase and caffeoyl CoA 3-O-methyltransferase in transgenic alfalfa. impacts on lignin structure and implications for the biosynthesis of G and S lignin. The Plant cell. 13:73-88.
Guo, H. S., J. F. Fei, Q. Xie, and N. H. Chua. 2003. A chemical-regulated inducible RNAi system in plants. The Plant journal: for cell and molecular biology. 34:383-392.
Hamilton, A. J., and D. C. Baulcombe. 1999. A species of small antisense RNA in posttranscriptional gene silencing in plants. Science. 286:950-952.
Hammond, S. M., E. Bernstein, D. Beach, and G. J. Hannon. 2000. An RNA-directed nuclease mediates post-transcriptional gene silencing in Drosophila cells. Nature. 404:293-296.
Hammond, S. M., S. Boettcher, A. A. Caudy, R. Kobayashi, and G. J. Hannon. 2001. Argonaute2, a link between genetic and biochemical analyses of RNAi. Science. 293:1146-1150.
Hannon, G. J. 2002. RNA interference. Nature. 418:244-251.
Helliwell, C., S. Wesley, and P. Waterhouse. 2005. Methods and means for producing efficient silencing constructs using recombinational cloning. Commonwealth Scientifific Industrial Research Corporation, US.
Herdewijn, P. 2000. Heterocyclic modifications of oligonucleotides and antisense technology. Antisense & nucleic acid drug development. 10:297-310.
Hudson, L., and F. C. Hay. 1980. Practical immunology. Blackwell Scientific; Blackwell Mosby Book Distributors, Oxford; Boston St. Louis, Mo. xvi, 359 p.
Hutvagner, G., and P. D. Zamore. 2002. A microRNA in a multiple-turnover RNAi enzyme complex. Science. 297:2056-2060.
Jackson, D., and e. al. 1980. Development of Sweet Sorghum as an Energy Crop, Volume 1: Agricultural Task. Battelle Laboratories Columbus Division.
Johnston, S., and D. Tang. 1994. Gene gun transfection of animal cells and genetic immunization. Methods Cell Biol. 43:353-365.
Jorgensen, R., C. Snyder, and J. Jones. 1987. T-DNA is organized predominantly in inverted repeat structures in plants transformed with Agrobacterium tumefaciens C58 derivatives. Mol. Gen. Genetics. 207:471-477.
Jung, J. H., W. M. Fouad, W. Vermerris, M. Gallo, and F. Altpeter. 2012. RNAi suppression of lignin biosynthesis in sugarcane reduces recalcitrance for biofuel production from lignocellulosic biomass. Plant biotechnology journal. 10:1067-1076.
Kennerdell, J. R., S. Yamaguchi, and R. W. Carthew. 2002. RNAi is activated during Drosophila oocyte maturation in a manner dependent on aubergine and spindle-E. Genes & development. 16:1884-1889.
Ketting, R. F., S. E. Fischer, E. Bernstein, T. Sijen, G. J. Hannon, and R. H. Plasterk. 2001. Dicer functions in RNA interference and in synthesis of small RNA involved in developmental timing in C. elegans. Genes & development. 15:2654-2659.
Kohler, G., and C. Milstein. 1975. Continuous cultures of fused cells secreting antibody of predefined specificity. Nature. 256:495-497.
Kozbor, D., P. Tripputi, J. C. Roder, and C. M. Croce. 1984. A human hybrid myeloma for production of human monoclonal antibodies. Journal of immunology. 133:3001-3005.
Kusaba, M. 2004. RNA interference in crop plants. Current opinion in biotechnology. 15:139-143.
Larkin, M. A., G. Blackshields, N. P. Brown, R. Chenna, P. A. McGettigan, H. McWilliam, F. Valentin, I. M. Wallace, A. Wilm, R. Lopez, J. D. Thompson, T. J. Gibson, and D. G. Higgins. 2007. Clustal W and Clustal X version 2.0. Bioinformatics. 23:2947-2948.
Llave, C., K. D. Kasschau, M. A. Rector, and J. C. Carrington. 2002. Endogenous and silencing-associated small RNAs in plants. The Plant cell. 14:1605-1619.
Lloyd, A., C. L. Plaisier, D. Carroll, and G. N. Drews. 2005. Targeted mutagenesis using zinc-finger nucleases in Arabidopsis. Proceedings of the National Academy of Sciences of the United States of America. 102:2232-2237.
Lu, F., and J. Ralph. 1997. Derivatization followed by reductive cleavage (DFRC method), a new method for lignin analysis: Protocol for analysis of DFRC monomers. J. Agric. Food Chem. 45:2590-2592.
Lu, L., M. Xiao, D. W. Clapp, Z. H. Li, and H. E. Broxmeyer. 1993. High efficiency retroviral mediated gene transduction into single isolated immature and replatable CD34(3+) hematopoietic stem/progenitor cells from human umbilical cord blood. The Journal of experimental medicine. 178:2089-2096.
Martinez, J., A. Patkaniowska, H. Urlaub, R. Luhrmann, and T. Tuschl. 2002. Single-stranded antisense siRNAs guide target RNA cleavage in RNAi. Cell. 110:563-574.
Monk, R. L. 1980. Improvements of Sorghum for Energy Production.
Nair, R. B., Q. Xia, C. J. Kartha, E. Kurylo, R. N. Hirji, R. Datla, and G. Selvaraj. 2002. Arabidopsis CYP98A3 mediating aromatic 3-hydroxylation. Developmental regulation of the gene, and expression in yeast. Plant physiology. 130:210-220.
Omirulleh, S., M. Abraham, M. Golovkin, I. Stefanov, M. K. Karabaev, L. Mustardy, S. Morocz, and D. Dudits. 1993. Activity of a chimeric promoter with the doubled CaMV 35S enhancer element in protoplast-derived cells and transgenic plants in maize. Plant molecular biology. 21:415-428.
Ossowski, S., R. Schwab, and D. Weigel. 2008. Gene silencing in plants using artificial microRNAs and other small RNAs. The Plant journal: for cell and molecular biology. 53:674-690.
Pal-Bhadra, M., U. Bhadra, and J. A. Birchler. 2002. RNAi related mechanisms affect both transcriptional and posttranscriptional transgene silencing in Drosophila. Molecular cell. 9:315-327.
Park, S.-H., C. Mei, M. Pauly, R. Ong, B. Dale, and R. Sabzikar. 2012. Downregulation of maize cinnamoyl-coenzyme a reductase via RNA interference technology causes brown midrib and improves ammonia fiber expansion-pretreated conversion into fermentable sugars for biofuels. Crop Sci. 52:2687-2701.
Park, W., J. Li, R. Song, J. Messing, and X. Chen. 2002. CARPEL FACTORY, a Dicer homolog, and HENT, a novel protein, act in microRNA metabolism in Arabidopsis thaliana. Current biology: CB. 12:1484-1495.
Parrish, S., and A. Fire. 2001. Distinct roles for RDE-1 and RDE-4 during RNA interference in Caenorhabditis elegans. Rna. 7:1397-1402.
Parrish, S., J. Fleenor, S. Xu, C. Mello, and A. Fire. 2000. Functional anatomy of a dsRNA trigger: differential requirement for the two trigger strands in RNA interference. Molecular cell. 6:1077-1087.
Preuss, S., and C. Pikaard. 2004. Targeted gene silencing in plants using RNA interference. In RNA Interference (RNAi)-Nuts & Bolts of siRNA Technology. D. Engelke, editor. DNA Press, Glendale, Calif. 23-36.
Raven, P. H., R. F. Evert, and S. E. Eichhorn. 1999. Biology of plants. W. H. Freeman: Worth Publishers, New York. xv, 944 p. pp.
Rhoades, M. W., B. J. Reinhart, L. P. Lim, C. B. Burge, B. Bartel, and D. P. Bartel. 2002. Prediction of plant microRNA targets. Cell. 110:513-520.
Rogers, S., H. Klee, R. Horsch, and R. Fraley. 1987. Improved vectors for plant transformation: expression cassette vectors and new selectable markers. Methods Enzymol. 153:253-277.
Rolando, C., B. Monties, and C. Lapierre. 1992. Thioacidolysis. In Methods in Lignin Chemistry. C. Dence and S. Lin, editors. springer-Verlag, Heidelberg. 334-349.
Saathoff, A. J., G. Sarath, E. K. Chow, B. S. Dien, and C. M. Tobias. 2011. Downregulation of cinnamyl-alcohol dehydrogenase in switchgrass by RNA silencing results in enhanced glucose release after cellulase treatment. PLoS One. 6:e16416.
Saballos, A., W. Vermerris, L. Rivera, and G. Ejeta. 2008. Allelic association, chemical characterization and saccharification properties of brown midrib mutants of sorghum (Sorghum bicolor (L.) Moench). Bioenergy Res. 1:193-204.
Sattler, S., D. Funnell-Harris, and J. Pedersen. 2010. Brown midrib mutations and their importance to the utilization of maize, sorghum, and pearl millet lignocellulosic tissues. Plant Sci. 178:229-238.
Sattler, S., N. Palmer, and A. Saballos. 2012. Identification and characterization of four missense mutations in brown midrib 12 (Bmr12), the Caffeic O-Methyltransferase (COMT) of sorghum. Bioenergy Res. 5:855-863.
Schook, L. B. 1987. Monoclonal antibody production techniques and applications. Dekker, New York. xiv, 316 p. pp.
Schwab, R., S. Ossowski, M. Riester, N. Warthmann, and D. Weigel. 2006. Highly specific gene silencing by artificial microRNAs in Arabidopsis. The Plant cell. 18:1121-1133.
Shahsavarani, H., D. Hasegawa, D. Yokota, M. Sugiyama, Y. Kaneko, C. Boonchird, and S. Harashima. 2013. Enhanced bio-ethanol production from cellulosic materials by semi-simultaneous saccharification and fermentation using high temperature resistant Saccharomyces cerevisiae TJ14. Journal of bioscience and bioengineering. 115:20-23.
Smith, N. A., S. P. Singh, M. B. Wang, P. A. Stoutjesdijk, A. G. Green, and P. M. Waterhouse. 2000. Total silencing by intron-spliced hairpin RNAs. Nature. 407:319-320.
Spielmann, A., and R. Simpson. 1986. T-DNA structgure in transgenic tobacco plants with multiple independent integration sites. Mol. Gen. Genetics. 205:34-41.
Tabara, H., M. Sarkissian, W. G. Kelly, J. Fleenor, A. Grishok, L. Timmons, A. Fire, and C. C. Mello. 1999. The rde-1 gene, RNA interference, and transposon silencing in C. elegans. Cell. 99:123-132.
Tijsterman, M., R. F. Ketting, and R. H. Plasterk. 2002. The genetics of RNA silencing. Annual review of genetics. 36:489-519.
Undersander, D., and W. Lane. 2001. Sorghums, sudangrasses, and sorghum-sudangrass hybrids. University of Wisconsin—Extension Cooperative Extension.
van der Weijde, T., C. L. Alvim Kamei, A. F. Tones, W. Vermerris, O. Dolstra, R. G. Visser, and L. M. Trindade. 2013. The potential of C4 grasses for cellulosic biofuel production. Frontiers in plant science. 4:107.
Vanholme, R., I. Cesarino, K. Rataj, Y. Xiao, L. Sundin, G. Goeminne, H. Kim, J. Cross, K. Morreel, P. Araujo, L. Welsh, J. Haustraete, C. McClellan, B. Vanholme, J. Ralph, G. G. Simpson, C. Halpin, and W. Boerjan. 2013. Caffeoyl shikimate esterase (CSE) is an enzyme in the lignin biosynthetic pathway in Arabidopsis. Science. 341:1103-1106.
Vermerris, W., A. Saballos, G. Ejeta, N. Mosier, M. Ladisch, and N. Carpita. 2007. Molecular breeding to enhance ethanol production from corn and sorghum stover. Crop Sci. 47:S-142-S-153.
Vogel, K., J. Pedersen, S. Masterson, and J. Toy. 1999. Evaluation of a filter bag system for NDF, ADF, and IVDMD forage analysis. Crop Sci. 39:276-279.
Voytas, D. F. 2013. Plant genome engineering with sequence-specific nucleases. Annual review of plant biology. 64:327-350.
Wagner, E., K. Zatloukal, M. Cotten, H. Kirlappos, K. Mechtler, D. T. Curiel, and M. L. Birnstiel. 1992. Coupling of adenovirus to transferrin-polylysine/DNA complexes greatly enhances receptor-mediated gene delivery and expression of transfected genes. Proceedings of the National Academy of Sciences of the United States of America. 89:6099-6103.
Warthmann, N., H. Chen, S. Ossowski, D. Weigel, and P. Herve. 2008. Highly specific silencing by artificial miRNAs in rice. PLoS One. 3:1-10.
Waterhouse, P. M., and C. A. Helliwell. 2003. Exploring plant genomes by RNA-induced gene silencing. Nature reviews. Genetics. 4:29-38.
Wesley, S. V., C. A. Helliwell, N. A. Smith, M. B. Wang, D. T. Rouse, Q. Liu, P. S. Gooding, S. P. Singh, D. Abbott, P. A. Stoutjesdijk, S. P. Robinson, A. P. Gleave, A. G. Green, and P. M. Waterhouse. 2001. Construct design for efficient, effective and high-throughput gene silencing in plants. The Plant journal: for cell and molecular biology. 27:581-590.
Williams, R. W., and G. M. Rubin. 2002. ARGONAUTE1 is required for efficient RNA interference in Drosophila embryos. Proceedings of the National Academy of Sciences of the United States of America. 99:6889-6894.
Wong, T. K., and E. Neumann. 1982. Electric field mediated gene transfer. Biochemical and biophysical research communications. 107:584-587.
Wu, L., M. Arakane, M. Ike, M. Wada, T. Takai, M. Gau, and K. Tokuyasu. 2011. Low temperature alkali pretreatment for improving enzymatic digestibility of sweet sorghum bagasse for ethanol production. Bioresource technology. 102:4793-4799.
Zamore, P. D., T. Tuschl, P. A. Sharp, and D. P. Bartel. 2000. RNAi: double-stranded RNA directs the ATP-dependent cleavage of mRNA at 21 to 23 nucleotide intervals. Cell. 101:25-33.
Zatloukal, K., E. Wagner, M. Cotten, S. Phillips, C. Plank, P. Steinlein, D. T. Curiel, and M. L. Birnstiel. 1992. Transferrinfection: a highly efficient way to express gene constructs in eukaryotic cells. Annals of the New York Academy of Sciences. 660:136-153.

### ADDITIONAL REFERENCES

### Sorted by Reference Number and Incorporated Herein by Reference in their Entireties

1. CRISPR/Cas, the Immune System of Bacteria and Archaea. Science. Vol. 327 no. 5962 pp. 167-170, (2010).
2. Biotechnology: Programming genomes with light. Nature. 500, 406-408, (2013).
3. CRISPR-Cas systems: beyond adaptive immunity. Nature Reviews Microbiology. 12, 317-326, (2014).
4. Targeted genome modification of crop plants using a CRISPR-Cas system. Nature Biotechnology. 31, 686-688, (2013).
5. Applying genotyping (TILLING) and phenotyping analyses to elucidate gene function in a chemically induced sorghum mutant population. BMC Plant Biology. 8, 103 (2008).
6. A combined biochemical screen and TILLING approach identifies mutations in Sorghum bicolor>Moench resulting in acyanogenic forage production. Plant Biotechnology Journal. 10, 54-66, (2012).

### CONCLUSION

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while process steps, formulation components or functions are presented in a given order, alternative embodiments may include these in a different order, or substantially concurrently. The teachings of the disclosure provided herein can be applied to other compositions, not only the compositions described herein. The various embodiments described herein can be combined to provide further embodiments.

Specific elements of any of the foregoing embodiments can be combined or substituted for elements in other embodiments. Furthermore, while aspects associated with certain embodiments of the disclosure have been described in the context of these embodiments, other embodiments may also exhibit such aspects, and not all embodiments need necessarily exhibit such aspects to fall within the scope of the disclosure. Accordingly, the disclosure is not limited, except as by the appended claims.

1-30. (canceled)

31. A method for converting a recessive trait to a dominant trait in a eukaryotic organism, the method comprising:
introducing into a cell of the eukaryotic organism a CRISPR-Cas vector system, wherein the CRISPR-Cas vector system is configured to generate a first guide sequence, a second guide sequence and a Cas endonuclease; and
introducing into the cell a donor arm comprising an antisense sequence of a first portion of a targeted sequence in a genomic locus of a DNA molecule encoding a targeted gene product having the recessive trait,
wherein the first guide sequence, the second guide sequence and the Cas endonuclease facilitate homologous recombination of the donor arm within the DNA molecule and at a location spaced apart from the first portion in a manner that modifies the DNA molecule, and
wherein expression of the modified DNA molecule is modified, thereby converting the recessive trait to the dominant trait.

32. A method for modifying expression of a targeted gene product in an eukaryotic cell, comprising:
introducing into the eukaryotic cell a vector system comprising one or more vectors comprising—
(a) a first regulatory element operably linked to a first guide sequence, wherein the first guide sequence hybridizes with a first target sequence in a genomic locus of a DNA molecule encoding the targeted gene product,
(b) a second regulatory element operably linked to a second guide sequence, wherein the second guide sequence hybridizes with a second target sequence in the genomic locus of the DNA molecule encoding the targeted gene product, and wherein the first target sequence is non-overlapping with the second target sequence, and
(c) a third regulatory element operably linked to a DNA sequence encoding a Cas endonuclease, wherein the Cas endonuclease induces double strand breaks at or near the first and second target sequences; and
introducing into the eukaryotic cell a donor arm comprising—
an antisense sequence of at least a portion of a targeted sequence in the genomic locus, wherein the portion of the targeted sequence is spaced apart from the first and second target sequences, and
a first homologous region and a second homologous region, wherein the first and second homologous regions flank the antisense sequence, and wherein the first homologous region hybridizes at or near the first target sequence and the second homologous region hybridizes at or near the second target sequence,
whereby, introduction of the vector system and the donor arm causes gene modification of the DNA molecule in a manner that modifies expression of the targeted gene product.

33. The method of claim 32 wherein gene modification includes homologous recombination of the donor arm and the DNA molecule at or near the first and second target sequences.

34. The method of claim 32 wherein gene modification of the DNA molecule generates a transcript having a hairpin structure.

35. The method of claim 32 wherein gene modification of the DNA molecule silences expression of a targeted gene product.

36. The method of claim 32 wherein the cell is a plant cell.

37. The method of claim 32 wherein the cell is a sorghum plant cell.

38. The method of claim 32 wherein the DNA molecule is at least one of sorghum sbCAD2 and sbCSE.
